SEQUENCE LISTING (14 sequences)

SEQ ID NO: 1 - DNA, 1140 bp, synthetic construct
  misc_feature 1..1140: note = Codon optimized GALT coding sequence
  source 1..1140: mol_type = other DNA
atgagccggagcggcaccgacccccagcagcggcagcaggccagcgaggccgacgctgccgccgccaccttccgggccaacgaccaccagcacatccggtacaaccccctgcaggacgagtgggtgctggtgagcgcccaccggatgaagcggccctggcagggccaggtggagccccagctgctgaagaccgtgccccggcacgaccccctgaaccctctgtgcccaggagccatccgcgccaacggcgaggtgaatcctcagtacgacagcaccttcctgttcgacaacgacttccccgccctgcagcccgacgcccccagccccggccccagcgaccatccattattccaggccaagagcgcccggggcgtgtgcaaggtgatgtgcttccacccctggagcgacgtgaccctgcccctgatgagcgtgcccgagatcagagccgtggtggacgcctgggccagcgtgaccgaggagctgggcgcccagtacccctgggtgcagatcttcgagaacaagggcgccatgatgggctgcagcaacccccacccccactgccaggtgtgggcctcaagcttcctgcccgacatcgcccagcgggaggagcggagccaacaggcctacaagtcacagcacggcgagcccctgctgatggagtacagccggcaggagctgctgcggaaggagcggctggtgctgaccagcgagcactggctcgtgctcgtgcccttctgggccacctggccctaccagaccctgctgctgcctagacggcacgtgcggcggctgcccgagctgacccccgccgagcgggacgacctggccagcatcatgaagaagctcctgaccaagtacgataacttattcgaaacaagcttcccctacagcatgggctggcacggcgcccccaccggctcagaggccggcgctaactggaaccactggcagctgcacgcccactactacccgcctctgctgagaagcgccaccgtgcggaagttcatggtgggctacgagatgctggcccaggcccagagagacttaacccctgagcaggccgccgagaggctccgggccttgccagaggtgcactaccacctgggacagaaggaccgggagacggccaccatcgcctga

SEQ ID NO: 2 - DNA, 260 bp, unidentified
  misc_feature 1..260: note = CB6 promoter
  source 1..260: mol_type = other DNA
ccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcggg

SEQ ID NO: 3 - DNA, 127 bp, unidentified
  misc_feature 1..127: note = rabbit beta-globin polyadenylation sequence
  source 1..127: mol_type = other DNA
gatctttttccctctgccaaaaattatggggacatcatgaagccccttgagcatctgacttctggctaataaaggaaatttattttcattgcaatagtgtgttggaattttttgtgtctctcactcg

SEQ ID NO: 4 - DNA, 382 bp, unidentified
  misc_feature 1..382: note = CMV-IE enhancer
  source 1..382: mol_type = other DNA
cgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattac

SEQ ID NO: 5 - skipped

SEQ ID NO: 6 - DNA, 105 bp, unidentified
  misc_feature 1..105: note = 5' ITR
  source 1..105: mol_type = other DNA
ctgcgcgctcgctcgctcactgaggccgcccgggcaaagcccgggcgtcgggcgacctttggtcgcccggcctcagtgagcgagcgagcgcgcagagagggagtg

SEQ ID NO: 7 - DNA, 130 bp, unidentified
  misc_feature 1..130: note = 3' ITR
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 8 - DNA, 2323 bp, synthetic construct
  misc_feature 1..2323: note = Full sequence of the AAV expression cassette
  source 1..2323: mol_type = other DNA
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcaattcacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctctagcctcgagaattcgccaccatgagccggagcggcaccgacccccagcagcggcagcaggccagcgaggccgacgctgccgccgccaccttccgggccaacgaccaccagcacatccggtacaaccccctgcaggacgagtgggtgctggtgagcgcccaccggatgaagcggccctggcagggccaggtggagccccagctgctgaagaccgtgccccggcacgaccccctgaaccctctgtgcccaggagccatccgcgccaacggcgaggtgaatcctcagtacgacagcaccttcctgttcgacaacgacttccccgccctgcagcccgacgcccccagccccggccccagcgaccatccattattccaggccaagagcgcccggggcgtgtgcaaggtgatgtgcttccacccctggagcgacgtgaccctgcccctgatgagcgtgcccgagatcagagccgtggtggacgcctgggccagcgtgaccgaggagctgggcgcccagtacccctgggtgcagatcttcgagaacaagggcgccatgatgggctgcagcaacccccacccccactgccaggtgtgggcctcaagcttcctgcccgacatcgcccagcgggaggagcggagccaacaggcctacaagtcacagcacggcgagcccctgctgatggagtacagccggcaggagctgctgcggaaggagcggctggtgctgaccagcgagcactggctcgtgctcgtgcccttctgggccacctggccctaccagaccctgctgctgcctagacggcacgtgcggcggctgcccgagctgacccccgccgagcgggacgacctggccagcatcatgaagaagctcctgaccaagtacgataacttattcgaaacaagcttcccctacagcatgggctggcacggcgcccccaccggctcagaggccggcgctaactggaaccactggcagctgcacgcccactactacccgcctctgctgagaagcgccaccgtgcggaagttcatggtgggctacgagatgctggcccaggcccagagagacttaacccctgagcaggccgccgagaggctccgggccttgccagaggtgcactaccacctgggacagaaggaccgggagacggccaccatcgcctgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 9 - skipped

SEQ ID NO: 10 - RNA, 13 nt, unidentified
  misc_feature 1..13: note = RNA Kozak sequence
  source 1..13: mol_type = other RNA
gccgccaccatgg

SEQ ID NO: 11-12 - skipped (2 entries)

SEQ ID NO: 13 - DNA, 5081 bp, synthetic construct
  misc_feature 1..5081: note = 3602 plasmid comprising the AAV expression cassette of SEQ ID NO:8
  source 1..5081: mol_type = other DNA
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcaattcacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctctagcctcgagaattcgccaccatgagccggagcggcaccgacccccagcagcggcagcaggccagcgaggccgacgctgccgccgccaccttccgggccaacgaccaccagcacatccggtacaaccccctgcaggacgagtgggtgctggtgagcgcccaccggatgaagcggccctggcagggccaggtggagccccagctgctgaagaccgtgccccggcacgaccccctgaaccctctgtgcccaggagccatccgcgccaacggcgaggtgaatcctcagtacgacagcaccttcctgttcgacaacgacttccccgccctgcagcccgacgcccccagccccggccccagcgaccatccattattccaggccaagagcgcccggggcgtgtgcaaggtgatgtgcttccacccctggagcgacgtgaccctgcccctgatgagcgtgcccgagatcagagccgtggtggacgcctgggccagcgtgaccgaggagctgggcgcccagtacccctgggtgcagatcttcgagaacaagggcgccatgatgggctgcagcaacccccacccccactgccaggtgtgggcctcaagcttcctgcccgacatcgcccagcgggaggagcggagccaacaggcctacaagtcacagcacggcgagcccctgctgatggagtacagccggcaggagctgctgcggaaggagcggctggtgctgaccagcgagcactggctcgtgctcgtgcccttctgggccacctggccctaccagaccctgctgctgcctagacggcacgtgcggcggctgcccgagctgacccccgccgagcgggacgacctggccagcatcatgaagaagctcctgaccaagtacgataacttattcgaaacaagcttcccctacagcatgggctggcacggcgcccccaccggctcagaggccggcgctaactggaaccactggcagctgcacgcccactactacccgcctctgctgagaagcgccaccgtgcggaagttcatggtgggctacgagatgctggcccaggcccagagagacttaacccctgagcaggccgccgagaggctccgggccttgccagaggtgcactaccacctgggacagaaggaccgggagacggccaccatcgcctgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgataactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 14 - protein, 379 aa, Homo sapiens
MSRSGTDPQQRQQASEADAAAATFRANDHQHIRYNPLQDEWVLVSAHRMKRPWQGQVEPQLLKTVPRHDPLNPLCPGAIRANGEVNPQYDSTFLFDNDFPALQPDAPSPGPSDHPLFQAKSARGVCKVMCFHPWSDVTLPLMSVPEIRAVVDAWASVTEELGAQYPWVQIFENKGAMMGCSNPHPHCQVWASSFLPDIAQREERSQQAYKSQHGEPLLMEYSRQELLRKERLVLTSEHWLVLVPFWATWPYQTLLLPRRHVRRLPELTPAERDDLASIMKKLLTKYDNLFETSFPYSMGWHGAPTGSEAGANWNHWQLHAHYYPPLLRSATVRKFMVGYEMLAQAQRDLTPEQAAERLRALPEVHYHLGQKDRETATIA

(19) United States
(12) Patent Application Publication
(10) Pub. No.: US 2025/0001008 A1
(43) Pub. Date: Jan. 2, 2025

(54) GENE THERAPY FOR GALACTOSEMIA

(71) Applicant: BridgeBio Gene Therapy Research, Inc., Raleigh, NC (US)

(72) Inventors: Clayton BEARD, Durham, NC (US); Julia Nicole CHAPMAN, Durham, NC (US); Daniel David MCCOY, Chapel Hill, NC (US)

(21) Appl. No.: 18/575,350

(22) PCT Filed: Jul. 1, 2022
(86) PCT No.: PCT/US2022/073389

§ 371 Date: Dec. 29, 2023

Related U.S. Application Data
(60) Provisional application No. 63/343,015, filed on May 17, 2022.
(60) Provisional application No. 63/217,456, filed on Jul. 1, 2021.

(51) Int. Cl. A61K 48/00; A61K 9/00; A61K 38/45; A61P 3/00; C12N 15/86

(52) U.S. Cl. CPC A61K 48/005; A61K 9/0019; A61K 38/45; A61K 48/0075; A61P 3/00; C12N 15/86; C12N 2750/14143; C12N 2750/14151; C12N 2830/15; C12N 2830/50

(57) ABSTRACT

The disclosure provides gene therapy compositions and methods for treating galactosemia (e.g., classic galactosemia). In particular, the disclosure provides compositions comprising recombinant adeno-associated viruses (rAAVs) comprising an AAV capsid protein, and an AAV expression cassette encoding Galactose-1-Phosphate Uridylyltransferase (GALT), and methods of use thereof.

### CROSS REFERENCE TO RELATED APPLICATIONS

The present application claims the benefit of priority under 35 U.S.C. § 119 (e) to U.S. Provisional Application No. 63/217,456, filed on Jul. 1, 2021, and U.S. Provisional Application No. 63/343,015, filed on May 17, 2022, the contents of each of which are hereby incorporated by reference in their entirety for all purposes.

### DESCRIPTION OF THE SEQUENCE LISTING XML FILE SUBMITTED ELECTRONICALLY

The contents of the Sequence Listing XML file submitted electronically herewith are incorporated herein by reference in their entirety: A Sequence Listing XML (filename: BGTR_002_02WO_SeqList_ST26.xml, date created: Jul. 1, 2022: file size: 23.4 kilobytes).

### BACKGROUND

Galactosemia is a severe, life-long, slowly-progressive, degenerative disorder. Due to acute neonatal toxicity most infants are identified through newborn screening within the first two weeks of life. Galactosemia is characterized by speech dyspraxia, difficulty in walking, and developmental delays in young children and adults, primary ovarian failure in females, and progressive motor and neurocognitive decline and dementia in adolescence and adulthood. The highest unmet needs include the long-term neurologic and ovarian complications.

Galactosemia can result from mutations in one of the three enzymes that are essential for galactose metabolism: galactose-1-phosphate uridyltransferase (GALT), galactokinase (GALK), and UDP-galactose-4-epimerase (GALE). GALT catalyzes the conversion of galactose-1-phosphate (Gal-1-P) and UDP-glucose to glucose-1-phosphate and UDP-galactose, respectively. Classic Galactosemia Type I and a milder form of galactosemia called variant Duarte galactosemia are caused by loss-of-function mutations in the GALT gene resulting in deficient GALT activity and thus toxic build-up of Gal-1-P and other galactose metabolites in the brain, liver, ovaries, and red blood cells (RBCs). Classic galactosemia occurs in 1 in 30,000 to 60,000 newborns in the United States, while Duarte galactosemia, which is characterized by less severe GALT deficiency, is more common and seen in about 1 in 4,000 newborns in the United States. Other types of galactosemia are less common.

Currently, there are no commercially-available therapies for galactosemia (e.g., classic galactosemia). The standard-of-care is dietary restriction to reduce exogenous galactose and/or lactose, which does very little to slow disease progression as endogenous galactose production still leads to accumulation of disease-progressing metabolites. Patients often undergo years of speech therapy, physical therapy, and occupational therapy. Most females undergo hormone replacement therapy. Patients usually face burdensome and life-long symptoms and complications despite a well-controlled diet. Even with early intervention long-term complications such as speech delay (56% of patients), intellectual deficits (45%), motor functions deficits (tremors & cerebellar ataxia) (18%), and primary ovarian insufficiency (POI) (91% of females) exist and highlight the urgent need for a therapy.

Thus, there is an unmet need for compositions and methods that can be used to treat galactosemia.

### SUMMARY

The present disclosure provides compositions and methods for use in the treatment of galactosemia (e.g., classic galactosemia). For example, the present disclosure provides nucleic acid molecules for use in the treatment of galactosemia, and compositions comprising the same. In some embodiments, a nucleic acid molecule provided herein comprises a sequence encoding Galactose-1-Phosphate Uridylyltransferase (GALT) or a sequence having substantial sequence identity (e.g., at least 90% sequence identity) to a GALT sequence, wherein the sequence is a component of an adeno-associated virus (AAV) expression cassette. In some embodiments, a nucleic acid molecule comprising an AAV expression cassette comprising a sequence encoding GALT or a sequence having substantial sequence identity (e.g., at least 90% sequence identity) to a GALT sequence is a component of a recombinant AAV (rAAV), which rAAV comprises a capsid protein (e.g., AAV9 capsid protein). The present disclosure also provides methods comprising administering a nucleic acid molecule, an rAAV, or a plasmid or cell comprising a nucleic acid molecule provided herein, or a pharmaceutical composition comprising the same, to a subject, such as a subject having galactosemia. In some embodiments, methods of treating galactosemia using a nucleic acid molecule, an rAAV, or a plasmid or cell comprising a nucleic acid molecule provided herein, or a pharmaceutical composition comprising the same, ameliorates, diminish the severity, eliminates, and/or delays the onset of one or more symptoms of galactosemia (e.g., as described herein). The present disclosure also provides methods of expressing GALT in a cell and methods of reducing levels of, e.g., galactose-1-phosphate (Gal-1-P) in a cell.

In an aspect, the present disclosure provides a nucleic acid molecule comprising an adeno-associated virus (AAV) expression cassette, wherein the AAV expression cassette comprises, from 5′ to 3′: a 5′ AAV inverted terminal repeat (ITR): a promoter: a transgene encoding Galactose-1-Phosphate Uridylyltransferase (GALT); and a 3′ AAV ITR.

In some embodiments, the transgene comprises a nucleic acid sequence having at least 90% identity (e.g., at least 91%, 92%, 93%, 94%, 95%, 96%, 97%, 98%, 99%, or greater) to SEQ ID NO: 1. In some embodiments, the transgene comprises the nucleic acid sequence of SEQ ID NO: 1. In some embodiments, the promoter is operably linked to the transgene. In some embodiments, the promoter comprises a CB6 promoter. In some embodiments, the CB6 promoter comprises the nucleic acid sequence of SEQ ID NO: 2. In some embodiments, the CB6 promoter comprises a nucleic acid sequence having at least 80%, 85%, 90%, 91%, 92%, 93%, 94%, 95%, 96%, 97%, 98%, 99%, or greater identity to SEQ ID NO: 2. In some embodiments, the AAV expression cassette comprises a nucleic acid sequence comprising a rabbit globin polyadenylation sequence. In some embodiments, the nucleic acid sequence comprising the rabbit globin polyadenylation sequence comprises the nucleic acid sequence of SEQ ID NO: 3. In some embodiments, the rabbit globin polyadenylation sequence comprises a nucleic acid sequence having at least 80%, 85%, 90%, 91%, 92%, 93%, 94%, 95%, 96%, 97%, 98%, 99%, or greater identity to SEQ ID NO: 3. In some embodiments, the AAV expression cassette comprises a CMV-IE enhancer. In some embodiments, the CMV-IE enhancer comprises the nucleic acid sequence of SEQ ID NO: 4. In some embodiments, the CMV-IE enhancer comprises a nucleic acid sequence having at least 80%, 85%, 90%, 91%, 92%, 93%, 94%, 95%, 96%, 97%, 98%, 99%, or greater identity to SEQ ID NO: 4. In some embodiments, the AAV expression cassette comprises a nucleic acid sequence comprising a Kozak sequence. In some embodiments, the nucleic acid sequence encoding the Kozak sequence comprises the nucleic acid sequence of SEQ ID NO: 5. In some embodiments, the Kozak sequence comprises a nucleic acid sequence having at least 80%, 85%, 90%, 91%, 92%, 93%, 94%, 95%, 96%, 97%, 98%, 99%, or greater identity to SEQ ID NO: 5. In some embodiments, the 5′ AAV ITR sequence comprises the nucleic acid sequence of SEQ ID NO: 6. In some embodiments, the 5′ AAV ITR sequence comprises a nucleic acid sequence having at least 80%, 85%, 90%, 91%, 92%, 93%, 94%, 95%, 96%, 97%, 98%, 99%, or greater identity to SEQ ID NO: 6. In some embodiments, the 3′ AAV ITR sequence comprises the nucleic acid sequence of SEQ ID NO: 7. In some embodiments, the 3′ AAV ITR sequence comprises a nucleic acid sequence having at least 80%, 85%, 90%, 91%, 92%, 93%, 94%, 95%, 96%, 97%, 98%, 99%, or greater identity to SEQ ID NO: 7. In some embodiments, the AAV expression cassette comprises, from 5′ to 3′: the 5′ AAV ITR, a CMV-IE enhancer, a CB6 promoter, a Kozak sequence, the transgene encoding GALT, a nucleic acid sequence encoding the rabbit globin polyadenylation sequence, and the 3′ AAV ITR. In some embodiments, the AAV expression cassette comprises the nucleic acid sequence of SEQ ID NO: 8. In some embodiments, the AAV expression cassette comprises a nucleic acid sequence having at least 80%, 85%, 90%, 91%, 92%, 93%, 94%, 95%, 96%, 97%, 98%, 99%, or greater identity to SEQ ID NO: 8.

The present disclosure also provides a plasmid comprising any one of the nucleic acid molecules disclosed herein, and a cell comprising any one of the nucleic acid molecules or plasmids disclosed herein.

In another aspect, the present disclosure provides a method of producing a recombinant adeno-associated virus (rAAV), the method comprising: contacting an AAV producer cell with any one of the nucleic acid molecules or plasmids disclosed herein. Accordingly, the present disclosure also provides an rAAV produced by a method disclosed herein. In some embodiments, the rAAV comprises an AAV9, AAV8, and/or AAVrh10 capsid protein. In some embodiments, the rAAV comprises an AAV9 capsid protein. The present disclosure also provides an rAAV comprising an AAV9 capsid protein and any of the nucleic acid molecules provided herein. In some embodiments, the rAAV is a self-complementary AAV (scAAV).

In a further aspect, the present disclosure provides an rAAV comprising: an AAV9 capsid protein; and any one of the nucleic acid molecules disclosed herein. In some embodiments, the nucleic acid molecule comprises an AAV expression cassette, wherein the AAV expression cassette comprises the nucleic acid sequence of SEQ ID NO: 8, or a sequence having at least 80%, 85%, 90%, 91%, 92%, 93%, 94%, 95%, 96%, 97%, 98%, 99%, or greater identity to SEQ ID NO: 8. In some embodiments, the nucleic acid molecule comprises an AAV expression cassette, wherein the AAV expression cassette comprises, from 5′ to 3′: a 5′ AAV ITR comprising the nucleic acid sequence of SEQ ID NO: 6, or a sequence having at least 80%, 85%, 90%, 91%, 92%, 93%, 94%, 95%, 96%, 97%, 98%, 99%, or greater identity to SEQ ID NO: 6: a CMV-IE enhancer comprising the nucleic acid sequence of SEQ ID NO: 4, or a sequence having at least 80%, 85%, 90%, 91%, 92%, 93%, 94%, 95%, 96%, 97%, 98%, 99%, or greater identity to SEQ ID NO: 4: a CB6 promoter comprising the nucleic acid sequence of SEQ ID NO: 2, or a sequence having at least 80%, 85%, 90%, 91%, 92%, 93%, 94%, 95%, 96%, 97%, 98%, 99%, or greater identity to SEQ ID NO: 2: a Kozak sequence comprising the nucleic acid sequence of SEQ ID NO: 5, or a sequence having at least 80%, 85%, 90%, 91%, 92%, 93%, 94%, 95%, 96%, 97%, 98%, 99%, or greater identity to SEQ ID NO: 5: the transgene encoding GALT comprising the nucleic acid sequence of SEQ ID NO: 1, or a sequence having at least 80%, 85%, 90%, 91%, 92%, 93%, 94%, 95%, 96%, 97%, 98%, 99%, or greater identity to SEQ ID NO: 1: a rabbit globin polyadenylation sequence comprising the nucleic acid sequence of SEQ ID NO: 3, or a sequence having at least 80%, 85%, 90%, 91%, 92%, 93%, 94%, 95%, 96%, 97%, 98%, 99%, or greater identity to SEQ ID NO: 3; and a 3′ AAV ITR comprising the nucleic acid sequence of SEQ ID NO: 7, or a sequence having at least 80%, 85%, 90%, 91%, 92%, 93%, 94%, 95%, 96%, 97%, 98%, 99%, or greater identity to SEQ ID NO: 7. In some embodiments, the rAAV is a self-complementary AAV.

In another aspect, the present disclosure provides a pharmaceutical composition comprising: any one of the nucleic acid molecules disclosed herein, any one of the plasmids disclosed herein, any one of the cells disclosed herein, or any one of the rAAVs disclosed herein; and a pharmaceutically acceptable carrier.

In a further aspect, the present disclosure provides a method of expressing Galactose-1-Phosphate Uridylyltransferase (GALT) in a cell, comprising: contacting the cell with any one of the nucleic acid molecules disclosed herein, any one of the plasmids disclosed herein, any one of the rAAVs disclosed herein, or any one of the compositions disclosed herein, thereby expressing GALT in the cell.

In another aspect, the present disclosure provides a method of reducing the level of galactose-1-phosphate (Gal-1-P) in a cell, comprising: contacting the cell with any one of the nucleic acid molecules disclosed herein, any one of the plasmids disclosed herein, any one of the rAAVs disclosed herein, or any one of the compositions disclosed herein, thereby reducing the level of Gal-1-P in the cell.

In some embodiments of the preceding methods, the contacting step is performed in vitro, ex vivo, or in vivo. In some embodiments of the preceding methods, the contacting step is performed in vivo in a subject in need thereof. In some embodiments of the preceding methods, the contacting step comprises administering a therapeutically effective amount of the nucleic acid molecule, the plasmid, the rAAV, or the composition to the subject. In some embodiments of the preceding methods, the cell is a brain cell, a liver cell, an ovarian cell, or a red blood cell (RBC).

In another aspect, the present disclosure provides a method of treating galactosemia in a subject in need thereof, comprising: administering to the subject a therapeutically effective amount of any one of the nucleic acid molecules disclosed herein, any one of the plasmids disclosed herein, any one of the rAAVs disclosed herein, or any one of the compositions disclosed herein, thereby treating galactosemia in the subject.

In some embodiments, the nucleic acid molecule, the plasmid, the cell, the rAAV, or the composition is administered intravenously. In some embodiments, the method comprises administering a therapeutically effective amount of rAAV, wherein the therapeutically effective amount is in a range of 10¹⁰ genome copies to 10¹⁴ genome copies per kilogram. In some embodiments, the administration results in an increase in the level of GALT in a cell of the subject, as compared to a control subject having galactosemia who is not administered the nucleic acid molecule, the plasmid, the cell, the rAAV, or the composition. In some embodiments, the administration results in an increase in GALT activity in a cell of the subject, as compared to a control subject having galactosemia who is not administered the nucleic acid molecule, the plasmid, the cell, the rAAV, or the composition. In some embodiments, the administration results in a decrease in the level of Gal-1-P in a cell of the subject, as compared to a control subject having galactosemia who is not administered the nucleic acid molecule, the plasmid, the cell, the rAAV, or the composition. In some embodiments, the cell is a brain cell, a liver cell, an ovarian cell, or a red blood cell (RBC). In some embodiments, the administration diminishes the severity of a symptom of galactosemia. In some embodiments, the administration delays the onset of a symptom of galactosemia. In some embodiments, the administration eliminates a symptom of galactosemia. In some embodiments, the symptom of galactosemia is liver dysfunction, kidney dysfunction, sepsis, brain edema, pseudotumor cerebri, feeding difficulty, reduction in growth, cataracts, speech disorder, language disorder, dyspraxia, developmental delay, attention deficit, learning delay, neurological impairment, behavioral disorder, emotional disorder, tremors, fertility dysfunction, primary ovarian insufficiency, anxiety, seizures, depression, attention deficit hyperactivity disorder (ADHD), dementia, or any combination thereof.

In some embodiments, the galactosemia is classic type I galactosemia. In some embodiments, the subject is a human subject. In some embodiments, the subject is a neonate or an infant. In some embodiments, the subject suffers from galactosemia. In some embodiments, the subject has been diagnosed with galactosemia. In some embodiments, the subject has one or more mutations in the GALT gene. In some embodiments, the subject is at a risk of developing at least one symptom of galactosemia.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a schematic depiction of an AAV expression cassette comprising from 5′ to 3″: (i) a 5′ AAV2-based ITR (e.g., a 5′ ITR comprising a nucleic acid sequence of SEQ ID NO: 6, or a sequence having at least 80%, 85%, 90%, 91%, 92%, 93%, 94%, 95%, 96%, 97%, 98%, 99%, or greater identity to SEQ ID NO: 6), (ii) a CMV-IE enhancer (e.g., a CMV-IE enhancer comprising a nucleic acid sequence of SEQ ID NO: 4, or a sequence having at least 80%, 85%, 90%, 91%, 92%, 93%, 94%, 95%, 96%, 97%, 98%, 99%, or greater identity to SEQ ID NO: 4), (iii) a CB6 promoter (e.g., a CB6 promoter comprising a nucleic acid sequence of SEQ ID NO: 2, or a sequence having at least 80%, 85%, 90%, 91%, 92%, 93%, 94%, 95%, 96%, 97%, 98%, 99%, or greater identity to SEQ ID NO: 2), (iv) a Kozak sequence (e.g., a Kozak sequence comprising a nucleic acid sequence of SEQ ID NO: 5, or a sequence having at least 80%, 85%, 90%, 91%, 92%, 93%, 94%, 95%, 96%, 97%, 98%, 99%, or greater identity to SEQ ID NO: 5), (v) a codon optimized transgene encoding human GALT protein (e.g., a codon optimized transgene of SEQ ID NO: 1 encoding human GALT protein, or a sequence having at least 80%, 85%, 90%, 91%, 92%, 93%, 94%, 95%, 96%, 97%, 98%, 99%, or greater identity to SEQ ID NO: 1), (vi) a rabbit globin polyadenylation sequence (e.g., a rabbit globin polyadenylation sequence comprising the nucleic acid sequence of SEQ ID NO: 3, or a sequence having at least 80%, 85%, 90%, 91%, 92%, 93%, 94%, 95%, 96%, 97%, 98%, 99%, or greater identity to SEQ ID NO: 3), and (vii) a 3′ AAV2-based ITR (e.g., a 3′ ITR comprising a nucleic acid sequence of SEQ ID NO: 7, or a sequence having at least 80%, 85%, 90%, 91%, 92%, 93%, 94%, 95%, 96%, 97%, 98%, 99%, or greater identity to SEQ ID NO: 7).

FIGS. 2A-2C show vector genome copies per μg of total genomic DNA of liver tissue (FIG. 2A), brain tissue (FIG. 2B), and ovarian tissue (FIG. 2C) of mice plotted against the dosage of AAV9-GALT administered to the mice. The data is shown for 4 weeks and 8 weeks post dosing, as indicated. Each dot represents an individual animal.

FIGS. 3A-3C show GALT messenger ribonucleic acid (mRNA) copies per μg of complementary deoxyribonucleic acid (cDNA) of liver tissue (FIG. 3A), brain tissue (FIG. 3B), and ovarian tissue (FIG. 3C) of mice plotted against the dosage of AAV9-GALT administered to the mice. The data is shown for 4 weeks and 8 weeks post dosing, as indicated. Each dot represents an individual animal.

FIG. 4 shows a graph of a ratio of GALT protein level to control GAPDH protein level (on the Y axis) plotted against the dosage of AAV9-GALT administered to the mice (on the X axis).

FIG. 5A shows a graph of a ratio of GALT protein level in brain tissue of the knockout mice to GALT protein level in control wildtype (WT) mice (on the Y axis) plotted against the dosage of AAV9-GALT administered to the mice (on the X axis). FIG. 5B shows a graph of a ratio of GALT protein level in liver tissue of the knockout mice to GALT protein level in control wildtype (WT) mice (on the Y axis) plotted again the dosage of AAV9-GALT administered to the mice (on the X axis).

FIG. 6 shows a diagram of an enzymatic assay for measuring galactose-1-phosphate (Gal-1-P) in tissue samples.

FIGS. 7A-7C show levels of Gal-1-P in liver tissue (FIG. 7A), brain tissue (FIG. 7B), and red blood cells (FIG. 7C) in mice injected intravenously with different doses of scAAV-GALT. The data is shown for 4 weeks and 8 weeks post dosing, as indicated. Each dot represents an individual animal.

FIG. 8A schematically illustrates a functional galactose metabolism (left panel) and a dysfunctional metabolism corresponding to loss of GALT enzyme activity (right panel). FIG. 8B schematically summarizes clinical manifestations of classic galactosemia in children, teens, and adults.

FIGS. 9A-9B show that scAAV-GALT particles restore galactose metabolism and reduces ER stress in Galt −/− HEK293T cells. FIG. 9A: Galt −/− HEK293T cells were propagated in hexose-free media supplemented with 1% glucose or 1% galactose. Some cells were transduced with AAV9-GFP or scAAV-GALT particles. After 72 hours, lysates were harvested and processed for western blot analysis for GRP78 (marker of ER stress), GALT, GFP, and GAPDH. FIG. 9B: Galt −/− cells were propagated in hexose-free media supplemented with 1% galactose. After 24 hours, cells were processed for gal-1P measurement by LC-MS/MS analysis.

FIGS. 10A-10B show vector genomes (FIG. 10A) and transgene RNA (FIG. 10B) copies per μg of cDNA of liver, brain, and ovary tissue after dosing of mice at 5.0×10¹² vg/kg. 2.5×10¹³ vg/kg, and 5.0×10¹³ vg/kg.

FIGS. 11A-11B show GALT protein expression in the brain (FIG. 11A) and liver (FIG. 11B) of Galt −/− mice, which are representative of mean±SEM.

FIGS. 12A-12C show gal-1P levels in brain (FIG. 12A), erythrocyte (FIG. 12B), and liver (FIG. 12C) of Galt −/− mice.

FIGS. 13A-13B show levels of vector genomes of scAAV-GALT particles in mouse brain (FIG. 13A) and liver (FIG. 13B) in a C57BI/6 mouse model. FIGS. 13C-13D show levels of Galt transgene in mouse brain (FIG. 13C) and liver (FIG. 13D) in the C57BI/6 mouse model.

FIGS. 14A-14B show litters per breeding pair (FIG. 14A) and time to pregnancy (FIG. 14B) for mice treated or untreated with scAAV-GALT particles. Relative GALT protein levels in the brain and liver are shown in FIGS. 14C and 14D. All data shown are mean±SEM of n=5 (WT) and n=6 (Galt −/− and Galt −/− with scAAV-GALT particles.

FIG. 15 shows the GALT reaction and stereochemistry of components evaluated in an in vitro potency assay.

FIG. 16 shows concentrations of mod-Gal-1P, Glu-UDP, mod-Gal-UDP, and Glu-1P as a function of time in various cell lines.

FIG. 17 shows Mod-Gal-UDP concentration as a function of time in various cell lines transduced with differing multiplicities of infection (MOIs).

FIG. 18 shows Mod-Gal-UDP concentration as a function of time with different mod-Gal-IP starting concentrations.

FIG. 19 shows Mod-Gal-UDP concentration as a function of time with different Glu-UDP starting concentrations.

FIGS. 20A-20B show GALT relative activity as a function of MOI in separate assays.

### DETAILED DESCRIPTION

The present disclosure provides gene therapy compositions and methods for treating galactosemia. In particular, the disclosure provides compositions, comprising recombinant adeno-associated viruses (rAAVs) comprising an AAV capsid protein, and an AAV expression cassette encoding Galactose-1-Phosphate Uridylyltransferase (GALT), and methods of use thereof.

It is to be understood that the terminology used herein is for the purpose of describing particular embodiments only and is not intended to be limiting.

Unless defined otherwise, all technical and scientific terms used herein have the same meaning as commonly understood to one of ordinary skill in the art to which the present application belongs. Although any methods and materials similar or equivalent to those described herein can be used in the practice or testing of the present application, representative methods and materials are herein described.

The terms “a”, “an”, and “the” refer to “one or more” when used in this application, including the claims. Thus, for example, reference to “a carrier” includes mixtures of one or more carriers, two or more carriers, and the like and reference to “the method” includes reference to equivalent steps and/or methods known to those skilled in the art, and so forth.

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 10%, such as plus or minus 5%.

Also as used herein, “and/or” refers to and encompasses any and all possible combinations of one or more of the associated listed items, as well as the lack of combinations when interpreted in the alternative (“or”). The use of the alternative (e.g., “or”) should be understood to mean either one, both, or any combination thereof of the alternatives.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like.

The term “pharmaceutically acceptable”, unless otherwise noted, is used to characterize a moiety (e.g., a salt, dosage form, or excipient) as being appropriate for use in accordance with sound medical judgment. In general, a pharmaceutically acceptable moiety has one or more benefits that outweigh any deleterious effect that the moiety may have. Deleterious effects may include, for example, excessive toxicity, irritation, allergic response, and other problems and complications.

As used herein, “treatment,” “treating.” “palliating,” and “ameliorating” are used interchangeably. These terms refer to an approach for obtaining beneficial or desired results including but not limited to a therapeutic benefit and/or a prophylactic benefit. Therapeutic benefit refers to any therapeutically relevant improvement in or effect on one or more diseases, conditions, or symptoms under treatment. The term “treating” in one embodiment, includes: (1) preventing or delaying the appearance of clinical symptoms of the state, disorder or condition developing in the patient that may be afflicted with or predisposed to the state, disorder or condition but does not yet experience or display clinical or subclinical symptoms of the state, disorder or condition: (2) inhibiting the state, disorder or condition (e.g., arresting, reducing or delaying the development of the disease, or a relapse thereof in case of maintenance treatment, of at least one clinical or subclinical symptom thereof): (3) relieving the condition (for example, by causing regression, or reducing the severity of the state, disorder or condition or at least one of its clinical or subclinical symptoms).

The term “effective amount” or “therapeutically effective amount” refers to the amount of an agent that is sufficient to achieve an outcome, for example, to effect beneficial or desired results, such as treatment of galactosemia (e.g., classic galactosemia) of a symptom thereof. The therapeutically effective amount may vary depending upon one or more of: the subject and disease condition being treated, the weight and age of the subject, the severity of the disease condition, the manner of administration and the like. A therapeutically effective amount may be an amount sufficient to treat galactosemia (e.g., classic galactosemia) and/or to ameliorate, diminish the severity of, eliminate, and/or delay the onset of one or more symptoms of galactosemia. In some embodiments, a therapeutically effective amount may be an amount sufficient to increase the activity of GALT in a subject, as determined by assessing GALT activity in a subject prior to administration of a therapeutic agent provided herein and after administration of the therapeutic agent.

The terms “subject.” “individual,” and “patient” are used interchangeably herein to refer to a vertebrate, such as a mammal. The mammal may be, for example, a mouse, a rat, a rabbit, a cat, a dog, a pig, a sheep, a horse, a non-human primate (e.g., cynomolgus monkey, chimpanzee), or a human. A subject's tissues, cells, or derivatives thereof, obtained in vivo or cultured in vitro are also encompassed. A human subject may be an adult, a teenager, a child (2 years to 14 years of age), an infant (1 month to 24 months), or a neonate (up to 1 month). In some embodiments, the adults are seniors about 65 years or older, or about 60 years or older.

In some embodiments, the subject is a pregnant woman or a woman intending to become pregnant.

An “adeno-associated virus (AAV) expression cassette” is a nucleic acid that gets packaged into a recombinant AAV vector, and comprises a sequence encoding one or more transgenes flanked by a 5′ inverted terminal repeat (ITR) and a 3′ITR.

As used herein, the terms “virus vector,” “viral vector,” or “gene delivery vector” refer to a virus particle that functions as a nucleic acid delivery vehicle, and which comprises a nucleic acid (e.g., an AAV expression cassette) packaged within a virion. Exemplary virus vectors include adeno-associated virus vectors (AAVs).

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAV type rh32.33, AAV type rh8, AAV type rh10, AAV type rh74, AAV type hu.68, avian AAV, bovine AAV, canine AAV, equine AAV, ovine AAV, snake AAV, bearded dragon AAV, AAV218, AAV2g9, AAV-LK03, AAV7m8, AAV Anc80, AAV PHP.B, and any other AAV now known or later discovered.

As used herein “sequence identity.” refers to the extent to which two optimally aligned polynucleotides or polypeptide sequences are invariant throughout a window of alignment of components, e.g. nucleotides or amino acids. An “identity fraction” for aligned segments of a test sequence and a reference sequence is the number of identical components which are shared by the two aligned sequences divided by the total number of components in the reference sequence segment, i.e. the entire reference sequence or a smaller defined part of the reference sequence. “Percent identity.” is the identity fraction times 100. The extent of identity (homology) between two sequences can be ascertained using a computer program and mathematical algorithm. Percentage identity can be calculated using the alignment program Clustal Omega, available at www.ebi.ac.uk/Tools/msa/clustalo using default parameters. See, Sievers et al., “Fast, scalable generation of high-quality protein multiple sequence alignments using Clustal Omega.” (2011 Oct. 11) Molecular systems biology 7:539. For the purposes of calculating identity to a sequence, extensions such as tags are not included.

As used herein, a nucleic acid sequence (e.g., coding sequence) and regulatory sequences are said to be “operably linked” when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein.

### AAV Expression Cassettes Encoding Galactose-1-Phosphate Uridylyltransferase (GALT)

The present disclosure provides gene therapy compositions and methods for treating galactosemia (e.g., classic galactosemia). For example, the present disclosure provides nucleic acid molecules comprising adeno-associated virus (AAV) expression cassettes. In some embodiments, the AAV expression cassette of a nucleic acid molecule comprises, from 5′ to 3″: a 5′ AAV inverted terminal repeat (ITR): a promoter: a transgene (e.g., a transgene encoding Galactose-1-Phosphate Uridylyltransferase (GALT)); and a 3′ AAV ITR.

In some embodiments, the AAV expression cassette comprises cis-acting 5′ and 3″ inverted terminal repeat sequences, as described further in B. J. Carter, in “Handbook of Parvoviruses”, ed., P. Tijsser, CRC Press, pp. 155 168 (1990), which is incorporated herein by reference in its entirety for all purposes. The AAV ITR sequences may be obtained from any known or presently unknown AAV, including presently identified mammalian AAV types disclosed herein.

In some embodiments, the AAV expression cassette comprises a 5′ ITR and/or a 3′ ITR derived from AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAV type rh32.33, AAV type rh8, AAV type rh10, AAV type rh74, AAV type hu.68, avian AAV, bovine AAV, canine AAV, equine AAV, ovine AAV, snake AAV, bearded dragon AAV, AAV218, AAV2g9, AAV-LK03, AAV7m8, AAV Anc80, or AAV PHP.B. In some embodiments, the AAV expression cassette comprises a 5′ ITR derived from AAV2, a 3′ ITR derived from AAV2, or a combination thereof.

In some embodiments, the 5′ AAV ITR sequence comprises a nucleic acid sequence having at least about 80% (for example, at least about 85%, about 90%, about 95%, about 96%, about 97%, about 98%, about 99%, about 99.5%, or greater, such as 100%, including all values and subranges that lie therebetween) identity to the sequence of SEQ ID NO: 6. In some embodiments, the 5′ AAV ITR sequence comprises, or consists of, the sequence of SEQ ID NO: 6.

In some embodiments, the 3′ AAV ITR sequence comprises a nucleic acid sequence having at least about 80% (for example, at least about 85%, about 90%, about 95%, about 96%, about 97%, about 98%, about 99%, about 99.5%, or greater, such as 100%, including all values and subranges that lie therebetween) identity to the sequence of SEQ ID NO: 7. In some embodiments, the 3′ AAV ITR sequence comprises, or consists of, the sequence of SEQ ID NO: 7.

In some embodiments, the AAV expression cassette comprises expression control elements which are operably linked to the transgene. Expression control elements include appropriate transcription initiation, termination, and enhancer sequences: efficient RNA processing signals such as splicing and polyadenylation (polyA) signals: sequences that stabilize cytoplasmic mRNA: sequences that enhance translation efficiency: sequences that enhance protein stability; and, in some cases, sequences that enhance secretion of the encoded product.

In some embodiments, the AAV expression cassette comprises an intron. In some embodiments, the intron is located between a promoter/enhancer sequence and a transgene. In some embodiments, the intron is derived from SV-40, and is referred to as the SV-40 T intron sequence. In some embodiments, the intron is a chimeric intron. In some embodiments, the AAV expression cassette comprises an internal ribosome entry site (IRES). In some embodiments, the AAV expression cassette comprises a nucleic acid encoding a 2A self-cleaving peptide. Illustrative 2A self-cleaving peptides include P2A, E2A, F2A, and T2A. In some embodiments, the AAV expression cassette comprises an element described in Sambrook et al., Molecular Cloning: A Laboratory Manual, Cold Spring Harbor Press, Cold Spring Harbor, N.Y., and references cited therein, at, for example, pages 3.18, 3.26, 16.17, and 16.27 and Ausubel et al., Current Protocols in Molecular Biology, John Wiley & Sons, New York, 1989, each of which is incorporated herein by reference in its entirety for all purposes.

In some embodiments, the AAV expression cassette comprises a woodchuck hepatitis virus post-transcriptional element (WPRE). (See, e.g., Wang and Verma, Proc. Natl. Acad. Sci., USA, 96:3906-3910 (1999)). In some embodiments, the AAV expression cassette comprises a hepatitis B virus posttranscriptional regulatory element (HBVPRE) and/or a RNA transport element (RTE). In some embodiments, the WPRE or HBVPRE sequence is any of the WPRE or HBVPRE sequences disclosed in U.S. Pat. Nos. 6,136,597 and 6,287,814.

In some embodiments, the AAV expression cassette comprises one or more 5′-non-transcribed and/or 5′-non-translated sequences involved with the initiation of transcription and translation, respectively, such as a TATA box, capping sequence, CAAT sequence, enhancer element, or the like. In some embodiments, the AAV expression cassette comprises an enhancer sequence and/or upstream activator sequence. In some embodiments, the AAV expression cassette comprises one or more 5′ leader and/or signal sequences.

In some embodiments, the AAV expression cassette comprises a constitutive promoter. Examples of constitutive promoters include, without limitation, the retroviral Rous sarcoma virus (RSV) LTR promoter (optionally with the RSV enhancer), the cytomegalovirus (CMV) promoter (optionally with the CMV enhancer), the CMV-IE enhancer, the SV40 promoter, the dihydrofolate reductase promoter, the β-actin promoter, the phosphoglycerol kinase (PGK) promoter, and the Efla promoter.

In some embodiments, the AAV expression cassette comprises an inducible promoter. Non-limiting examples of inducible promoters include the zinc-inducible sheep metallothionine (MT) promoter, the dexamethasone (Dex)-inducible mouse mammary tumor virus (MMTV) promoter, the T7 polymerase promoter system, the ecdysone insect promoter, the tetracycline-repressible system, the tetracycline-inducible system, the RU486-inducible system, and the rapamycin-inducible system. Other types of inducible promoters include those that are regulated by a specific physiological state, e.g., temperature, acute phase, a particular differentiation state of the cell, or a specific cell cycle phase.

In some embodiments, the AAV expression cassette comprises the native promoter, or fragment thereof, or the native expression control element, operably linked to the transgene encoding GALT. In some embodiments, the AAV expression cassette comprises one or more regulatory sequences that impart tissue-specific gene expression capabilities (e.g., tissue-specific regulatory sequences). In some cases, a tissue-specific regulatory sequence binds one or more tissue-specific transcription factors that induce transcription in a tissue-specific manner. Examples of tissue-specific regulatory sequences include, but are not limited to, the following tissue specific promoters: neuronal promoters such as the neuron-specific enolase (NSE) promoter, the neurofilament light chain gene promoter, and the neuron-specific vgf gene promoter.

In some embodiments, the AAV expression cassette comprises one or more promoters. In some embodiments, the AAV expression cassette comprises a chicken β-actin promoter. In some embodiments, the AAV expression cassette comprises a CB6 promoter. In some embodiments, the CB6 promoter comprises a nucleic acid sequence having at least about 80% (for example, at least about 85%, about 90%, about 95%, about 96%, about 97%, about 98%, about 99%, about 99.5%, or greater, such as 100%, including all values and subranges that lie therebetween) identity to the sequence of SEQ ID NO: 2. In some embodiments, the CB6 promoter comprises, or consists of, the nucleic acid sequence of SEQ ID NO: 2.

In some embodiments, the AAV expression cassette comprises a CMV-IE enhancer. In some embodiments, the enhancer is a CMV-IE enhancer. In some embodiments, the CMV-IE enhancer comprises a nucleic acid sequence having at least about 80% (for example, at least about 85%, about 90%, about 95%, about 96%, about 97%, about 98%, about 99%, about 99.5%, or greater, such as 100%, including all values and subranges that lie therebetween) identity to the sequence of SEQ ID NO: 4. In some embodiments, the CMV-IE enhancer comprises, or consists of, the nucleic acid sequence of SEQ ID NO: 4.

In some embodiments, the AAV expression cassette comprises a CAG promoter. In some embodiments, a CAG promoter comprises a CMV-IE enhancer (e.g., as described herein) and a chicken β-actin promoter (e.g., as described herein). In some embodiments, the CAG promoter further comprises a chimeric intron.

In some embodiments, the AAV expression cassette comprises a consensus sequence, such as a Kozak sequence (for example, a DNA sequence transcribed to an RNA Kozak sequence). As used herein, a “Kozak sequence” refers to a DNA element encoding an “RNA Kozak sequence” which regulates translational initiation.” In some embodiments, the AAV expression cassette comprises a Kozak sequence. In some embodiments, the Kozak sequence comprises a nucleic acid sequence having at least about 80% (for example, at least about 85%, about 90%, about 95%, about 96%, about 97%, about 98%, about 99%, about 99.5%, or greater, such as 100%, including all values and subranges that lie therebetween) identity to the sequence of SEQ ID NO: 5. In some embodiments, the Kozak sequence comprises, or consists of, the nucleic acid sequence of SEQ ID NO: 5.

In some embodiments, the AAV expression cassette comprises a Kozak sequence upstream of the transgene. In some embodiments, the Kozak sequence (e.g., RNA Kozak sequence) comprises or consists of ACCAUGG (SEQ ID NO: 9), GCCGCCACCAUGG (SEQ ID NO: 10), CCACCAUG (SEQ ID NO: 11), or CCACCAUGG (SEQ ID NO: 12).

In some embodiments, the AAV expression cassette comprises one or more binding sites for one or more miRNAs. In some embodiments, the AAV expression cassette comprises an miRNA binding site that is capable of regulating tissue specific expression of the GALT transgene. In some embodiments, the miRNA binding site that is capable of regulating tissue specific expression of the GALT transgene is an miR-122 binding site, an miR-133a, or a miR-1 binding site. For example, expression of the GALT transgene in the liver may be inhibited by incorporating a binding site for miR-122 such that mRNA expressed from the transgene binds to and is inhibited by miR-122 in the liver. Expression of the GALT transgene in the heart may be inhibited by incorporating a binding site for miR-133a or miR-1, such that mRNA expressed from the transgene binds to and is inhibited by miR-133a or miR-1 in the heart. In some embodiments, miRNA target sites in mRNA are in the 5′ untranslated region (UTR), the 3′ UTR, or in the coding region. Furthermore, the GALT transgene may be designed such that multiple miRNAs regulate mRNA by recognizing the same or multiple sites. The presence of multiple miRNA binding sites may result in the cooperative action of multiple RNA-induced silencing complexes (RISCs) and provide highly efficient inhibition of expression. The target site sequence may comprise a total of at least 5, 10, or more nucleotides, such as between 5-100, or between 10-60 nucleotides. The target site sequence may comprise at least 5 nucleotides of the sequence of a target gene binding site. In some embodiments, the AAV expression cassette comprises an miR-1 binding site, an miR-133a binding site, an miR-122 binding site, or any combination thereof.

In some embodiments, the AAV expression cassette comprises a polyadenylation (polyA) sequence. As used herein, the “poly A sequence” refers to a DNA sequence that when transcribed regulates the addition of a polyA tail to the mRNA transcript. Poly A signals may be derived from many suitable species, including, without limitation SV-40, human, and bovine. In some embodiments, the polyA sequence is a β-globin polyA sequence, such as a mammalian β-globin poly A sequence. In some embodiments, the polyA sequence is a human polyA sequence or a bovine β-globin polyA sequence. In some embodiments, the AAV expression cassette comprises a rabbit β-globin polyA sequence. In some embodiments, the rabbit β-globin polyA sequence comprises the nucleic acid sequence of SEQ ID NO: 3, or a sequence having at least about 80% (for example, at least about 85%, about 90%, about 95%, about 96%, about 97%, about 98%, about 99%, about 99.5%, or greater, such as 100%, including all values and subranges that lie therebetween) identity to the sequence of SEQ ID NO: 3.

In some embodiments, the transgene comprises a codon-optimized sequence encoding the GALT protein. In some embodiments, the transgene comprises a codon-optimized nucleic acid sequence having at least about 80% (for example, at least about 85%, about 90%, about 95%, about 96%, about 97%, about 98%, about 99%, about 99.5%, or greater, such as 100%, including all values and subranges that lie therebetween) identity to the sequence of SEQ ID NO: 1. In some embodiments, the transgene comprises a codon-optimized sequence comprising or consisting of the nucleic acid sequence of SEQ ID NO: 1.

In some embodiments, the transgene comprises a codon-optimized sequence encoding the GALT protein, wherein the GALT protein comprises an amino acid sequence with at least about 80% (for example, at least about 85%, about 90%, about 95%, about 96%, about 97%, about 98%, about 99%, about 99.5%, or greater, such as 100%, including all values and subranges that lie therebetween) identity to the amino acid sequence of SEQ ID NO: 14. In some embodiments, the transgene comprises a codon-optimized sequence encoding the GALT protein, wherein the GALT protein comprises, or consists of, an amino acid sequence of SEQ ID NO: 14.

In some embodiments, the AAV expression cassette comprises, from 5′ to 3″: (i) a 5′ AAV2-based ITR, (ii) a CMV-IE enhancer, (iii) a CB6 promoter, (iv) a Kozak sequence, (v) a codon optimized transgene encoding human GALT protein, (vi) a rabbit globin polyadenylation sequence, and (vii) a 3′ AAV2-based ITR. In some embodiments, the AAV expression cassette comprises, from 5′ to 3″: (i) a 5′ AAV2-based ITR, (ii) a CMV-IE enhancer, (iii) a CB6 promoter, (iv) a Kozak sequence, (v) a codon optimized transgene of SEQ ID NO: 1 encoding human GALT protein, (vi) a rabbit globin polyadenylation sequence, and (vii) a 3″ AAV2-based ITR.

In some embodiments, the AAV expression cassette comprises, from 5′ to 3″: (i) a 5′ AAV2-based ITR comprising a nucleic acid sequence of SEQ ID NO: 6; (ii) a CMV-IE enhancer: (iii) a CB6 promoter: (iv) a Kozak sequence: (v) a codon optimized transgene of SEQ ID NO: 1 encoding human GALT protein: (vi) a rabbit globin polyadenylation sequence; and (vii) a 3′ AAV2-based ITR. In some embodiments, the AAV expression cassette comprises, from 5′ to 3″: (i) a 5′ AAV2-based ITR: (ii) a CMV-IE enhancer: (iii) a CB6 promoter: (iv) a Kozak sequence: (v) a codon optimized transgene of SEQ ID NO: 1 encoding human GALT protein: (vi) a rabbit globin polyadenylation sequence; and (vii) a 3′ AAV2-based ITR comprising a nucleic acid sequence of SEQ ID NO: 7. In some embodiments, the AAV expression cassette comprises, from 5′ to 3″: (i) a 5′ AAV2-based ITR comprising a nucleic acid sequence of SEQ ID NO: 6: (ii) a CMV-IE enhancer: (iii) a CB6 promoter: (iv) a Kozak sequence: (v) a codon optimized transgene of SEQ ID NO: 1 encoding human GALT protein: (vi) a rabbit globin polyadenylation sequence; and (vii) a 3′ AAV2-based ITR comprising a nucleic acid sequence of SEQ ID NO: 7. In some embodiments, the AAV expression cassette comprises, from 5′ to 3″: (i) a 5′ AAV2-based ITR comprising a nucleic acid sequence of SEQ ID NO: 6, or a sequence having at least 80%, 85%, 90%, 91%, 92%, 93%, 94%, 95%, 96%, 97%, 98%, 99%, or greater identity to SEQ ID NO: 6: (ii) a CMV-IE enhancer: (iii) a CB6 promoter: (iv) a Kozak sequence: (v) a codon optimized transgene of SEQ ID NO: 1 encoding human GALT protein, or a sequence having at least 80%, 85%, 90%, 91%, 92%, 93%, 94%, 95%, 96%, 97%, 98%, 99%, or greater identity to SEQ ID NO: 1: (vi) a rabbit globin polyadenylation sequence; and (vii) a 3′ AAV2-based ITR. In some embodiments, the AAV expression cassette comprises, from 5′ to 3″: (i) a 5′ AAV2-based ITR: (ii) a CMV-IE enhancer: (iii) a CB6 promoter: (iv) a Kozak sequence: (v) a codon optimized transgene of SEQ ID NO: 1 encoding human GALT protein, or a sequence having at least 80%, 85%, 90%, 91%, 92%, 93%, 94%, 95%, 96%, 97%, 98%, 99%, or greater identity to SEQ ID NO: 1: (vi) a rabbit globin polyadenylation sequence; and (vii) a 3′ AAV2-based ITR comprising a nucleic acid sequence of SEQ ID NO: 7, or a sequence having at least 80%, 85%, 90%, 91%, 92%, 93%, 94%, 95%, 96%, 97%, 98%, 99%, or greater identity to SEQ ID NO: 7. In some embodiments, the AAV expression cassette comprises, from 5′ to 3″: (i) a 5′ AAV2-based ITR comprising a nucleic acid sequence of SEQ ID NO: 6, or a sequence having at least 80%, 85%, 90%, 91%, 92%, 93%, 94%, 95%, 96%, 97%, 98%, 99%, or greater identity to SEQ ID NO: 6; (ii) a CMV-IE enhancer: (iii) a CB6 promoter: (iv) a Kozak sequence: (v) a codon optimized transgene of SEQ ID NO: 1 encoding human GALT protein, or a sequence having at least 80%, 85%, 90%, 91%, 92%, 93%, 94%, 95%, 96%, 97%, 98%, 99%, or greater identity to SEQ ID NO: 1: (vi) a rabbit globin polyadenylation sequence; and (vii) a 3′ AAV2-based ITR comprising a nucleic acid sequence of SEQ ID NO: 7, or a sequence having at least 80%, 85%, 90%, 91%, 92%, 93%, 94%, 95%, 96%, 97%, 98%, 99%, or greater identity to SEQ ID NO: 7.

In some embodiments, the AAV expression cassette comprises, from 5′ to 3″: (i) a 5′ AAV2-based ITR: (ii) a CMV-IE enhancer comprising a nucleic acid sequence of SEQ ID NO: 4: (iii) a CB6 promoter: (iv) a Kozak sequence: (v) a codon optimized transgene of SEQ ID NO: 1 encoding human GALT protein: (vi) a rabbit globin polyadenylation sequence; and (vii) a 3′ AAV2-based ITR. In some embodiments, the AAV expression cassette comprises, from 5′ to 3″: (i) a 5′ AAV2-based ITR: (ii) a CMV-IE enhancer: (iii) a CB6 promoter comprising a nucleic acid sequence of SEQ ID NO: 2: (iv) a Kozak sequence: (v) a codon optimized transgene of SEQ ID NO: 1 encoding human GALT protein: (vi) a rabbit globin polyadenylation sequence; and (vii) a 3′ AAV2-based ITR. In some embodiments, the AAV expression cassette comprises, from 5′ to 3″: (i) a 5′ AAV2-based ITR: (ii) a CMV-IE enhancer comprising a nucleic acid sequence of SEQ ID NO: 4: (iii) a CB6 promoter comprising a nucleic acid sequence of SEQ ID NO: 2: (iv) a Kozak sequence: (v) a codon optimized transgene of SEQ ID NO: 1 encoding human GALT protein: (vi) a rabbit globin polyadenylation sequence; and (vii) a 3′ AAV2-based ITR. In some embodiments, the AAV expression cassette comprises, from 5° to 3″: (i) a 5′ AAV2-based ITR: (ii) a CMV-IE enhancer comprising a nucleic acid sequence of SEQ ID NO: 4, or a sequence having at least 80%, 85%, 90%, 91%, 92%, 93%, 94%, 95%, 96%, 97%, 98%, 99%, or greater identity to SEQ ID NO: 4: (iii) a CB6 promoter: (iv) a Kozak sequence: (v) a codon optimized transgene of SEQ ID NO: 1 encoding human GALT protein, or a sequence having at least 80%, 85%, 90%, 91%, 92%, 93%, 94%, 95%, 96%, 97%, 98%, 99%, or greater identity to SEQ ID NO: 1: (vi) a rabbit globin polyadenylation sequence; and (vii) a 3′ AAV2-based ITR. In some embodiments, the AAV expression cassette comprises, from 5′ to 3″: (i) a 5′ AAV2-based ITR: (ii) a CMV-IE enhancer: (iii) a CB6 promoter comprising a nucleic acid sequence of SEQ ID NO: 2, or a sequence having at least 80%, 85%, 90%, 91%, 92%, 93%, 94%, 95%, 96%, 97%, 98%, 99%, or greater identity to SEQ ID NO: 2: (iv) a Kozak sequence: (v) a codon optimized transgene of SEQ ID NO: 1 encoding human GALT protein, or a sequence having at least 80%, 85%, 90%, 91%, 92%, 93%, 94%, 95%, 96%, 97%, 98%, 99%, or greater identity to SEQ ID NO: 1: (vi) a rabbit globin polyadenylation sequence; and (vii) a 3′ AAV2-based ITR. In some embodiments, the AAV expression cassette comprises, from 5′ to 3″: (i) a 5′ AAV2-based ITR: (ii) a CMV-IE enhancer comprising a nucleic acid sequence of SEQ ID NO: 4, or a sequence having at least 80%, 85%, 90%, 91%, 92%, 93%, 94%, 95%, 96%, 97%, 98%, 99%, or greater identity to SEQ ID NO: 4: (iii) a CB6 promoter comprising a nucleic acid sequence of SEQ ID NO: 2, or a sequence having at least 80%, 85%, 90%, 91%, 92%, 93%, 94%, 95%, 96%, 97%, 98%, 99%, or greater identity to SEQ ID NO: 2: (iv) a Kozak sequence: (v) a codon optimized transgene of SEQ ID NO: 1 encoding human GALT protein, or a sequence having at least 80%, 85%, 90%, 91%, 92%, 93%, 94%, 95%, 96%, 97%, 98%, 99%, or greater identity to SEQ ID NO: 1: (vi) a rabbit globin polyadenylation sequence; and (vii) a 3′ AAV2-based ITR.

In some embodiments, the AAV expression cassette comprises, from 5′ to 3″: (i) a 5′ AAV2-based ITR: (ii) a CMV-IE enhancer: (iii) a CB6 promoter: (iv) a Kozak sequence comprising a nucleic acid sequence of SEQ ID NO: 5: (v) a codon optimized transgene of SEQ ID NO: 1 encoding human GALT protein: (vi) a rabbit globin polyadenylation sequence; and (vii) a 3′ AAV2-based ITR. In some embodiments, the AAV expression cassette comprises, from 5′ to 3″: (i) a 5′ AAV2-based ITR: (ii) a CMV-IE enhancer: (iii) a CB6 promoter: (iv) a Kozak sequence: (v) a codon optimized transgene of SEQ ID NO: 1 encoding human GALT protein: (vi) a rabbit globin polyadenylation sequence comprising the nucleic acid sequence of SEQ ID NO: 3; and (vii) a 3′ AAV2-based ITR. In some embodiments, the AAV expression cassette comprises, from 5′ to 3″: (i) a 5′ AAV2-based ITR: (ii) a CMV-IE enhancer comprising a nucleic acid sequence of SEQ ID NO: 4: (iii) a CB6 promoter comprising a nucleic acid sequence of SEQ ID NO: 2: (iv) a Kozak sequence comprising a nucleic acid sequence of SEQ ID NO: 5: (v) a codon optimized transgene of SEQ ID NO: 1 encoding human GALT protein: (vi) a rabbit globin polyadenylation sequence comprising the nucleic acid sequence of SEQ ID NO: 3; and (vii) a 3′ AAV2-based ITR comprising a nucleic acid sequence. In some embodiments, the AAV expression cassette comprises, from 5′ to 3″: (i) a 5′ AAV2-based ITR: (ii) a CMV-IE enhancer: (iii) a CB6 promoter: (iv) a Kozak sequence comprising a nucleic acid sequence of SEQ ID NO: 5, or a sequence having at least 80%, 85%, 90%, 91%, 92%, 93%, 94%, 95%, 96%, 97%, 98%, 99%, or greater identity to SEQ ID NO: 5: (v) a codon optimized transgene of SEQ ID NO: 1 encoding human GALT protein, or a sequence having at least 80%, 85%, 90%, 91%, 92%, 93%, 94%, 95%, 96%, 97%, 98%, 99%, or greater identity to SEQ ID NO: 1: (vi) a rabbit globin polyadenylation sequence; and (vii) a 3″ AAV2-based ITR. In some embodiments, the AAV expression cassette comprises, from 5′ to 3″: (i) a 5′ AAV2-based ITR: (ii) a CMV-IE enhancer: (iii) a CB6 promoter: (iv) a Kozak sequence: (v) a codon optimized transgene of SEQ ID NO: 1 encoding human GALT protein, or a sequence having at least 80%, 85%, 90%, 91%, 92%, 93%, 94%, 95%, 96%, 97%, 98%, 99%, or greater identity to SEQ ID NO: 1: (vi) a rabbit globin polyadenylation sequence comprising the nucleic acid sequence of SEQ ID NO: 3, or a sequence having at least 80%, 85%, 90%, 91%, 92%, 93%, 94%, 95%, 96%, 97%, 98%, 99%, or greater identity to SEQ ID NO: 3; and (vii) a 3″ AAV2-based ITR. In some embodiments, the AAV expression cassette comprises, from 5′ to 3″: (i) a 5′ AAV2-based ITR: (ii) a CMV-IE enhancer comprising a nucleic acid sequence of SEQ ID NO: 4, or a sequence having at least 80%, 85%, 90%, 91%, 92%, 93%, 94%, 95%, 96%, 97%, 98%, 99%, or greater identity to SEQ ID NO: 4: (iii) a CB6 promoter comprising a nucleic acid sequence of SEQ ID NO: 2, or a sequence having at least 80%, 85%, 90%, 91%, 92%, 93%, 94%, 95%, 96%, 97%, 98%, 99%, or greater identity to SEQ ID NO: 2: (iv) a Kozak sequence comprising a nucleic acid sequence of SEQ ID NO: 5, or a sequence having at least 80%, 85%, 90%, 91%, 92%, 93%, 94%, 95%, 96%, 97%, 98%, 99%, or greater identity to SEQ ID NO: 5: (v) a codon optimized transgene of SEQ ID NO: 1 encoding human GALT protein, or a sequence having at least 80%, 85%, 90%, 91%, 92%, 93%, 94%, 95%, 96%, 97%, 98%, 99%, or greater identity to SEQ ID NO: 1: (vi) a rabbit globin polyadenylation sequence comprising the nucleic acid sequence of SEQ ID NO: 3, or a sequence having at least 80%, 85%, 90%, 91%, 92%, 93%, 94%, 95%, 96%, 97%, 98%, 99%, or greater identity to SEQ ID NO: 3; and (vii) a 3′ AAV2-based ITR comprising a nucleic acid sequence.

In some embodiments, the AAV expression cassette comprises, from 5′ to 3″: (i) a 5′ AAV2-based ITR comprising a nucleic acid sequence of SEQ ID NO: 6; (ii) a CMV-IE enhancer comprising a nucleic acid sequence of SEQ ID NO: 4: (iii) a CB6 promoter comprising a nucleic acid sequence of SEQ ID NO: 2: (iv) a Kozak sequence comprising a nucleic acid sequence of SEQ ID NO: 5: (v) a codon optimized transgene of SEQ ID NO: 1 encoding human GALT protein: (vi) a rabbit globin polyadenylation sequence comprising the nucleic acid sequence of SEQ ID NO: 3; and (vii) a 3′ AAV2-based ITR comprising a nucleic acid sequence of SEQ ID NO: 7. In some embodiments, the AAV expression cassette comprises, from 5′ to 3″: (i) a 5′ AAV2-based ITR comprising a nucleic acid sequence of SEQ ID NO: 6, or a sequence having at least 80%, 85%, 90%, 91%, 92%, 93%, 94%, 95%, 96%, 97%, 98%, 99%, or greater identity to SEQ ID NO: 6: (ii) a CMV-IE enhancer comprising a nucleic acid sequence of SEQ ID NO: 4, or a sequence having at least 80%, 85%, 90%, 91%, 92%, 93%, 94%, 95%, 96%, 97%, 98%, 99%, or greater identity to SEQ ID NO: 4: (iii) a CB6 promoter comprising a nucleic acid sequence of SEQ ID NO: 2, or a sequence having at least 80%, 85%, 90%, 91%, 92%, 93%, 94%, 95%, 96%, 97%, 98%, 99%, or greater identity to SEQ ID NO: 2: (iv) a Kozak sequence comprising a nucleic acid sequence of SEQ ID NO: 5, or a sequence having at least 80%, 85%, 90%, 91%, 92%, 93%, 94%, 95%, 96%, 97%, 98%, 99%, or greater identity to SEQ ID NO: 5: (v) a codon optimized transgene of SEQ ID NO: 1 encoding human GALT protein, or a sequence having at least 80%, 85%, 90%, 91%, 92%, 93%, 94%, 95%, 96%, 97%, 98%, 99%, or greater identity to SEQ ID NO: 1: (vi) a rabbit globin polyadenylation sequence comprising the nucleic acid sequence of SEQ ID NO: 3, or a sequence having at least 80%, 85%, 90%, 91%, 92%, 93%, 94%, 95%, 96%, 97%, 98%, 99%, or greater identity to SEQ ID NO: 3; and (vii) a 3′ AAV2-based ITR comprising a nucleic acid sequence of SEQ ID NO: 7, or a sequence having at least 80%, 85%, 90%, 91%, 92%, 93%, 94%, 95%, 96%, 97%, 98%, 99%, or greater identity to SEQ ID NO: 7.

In some embodiments, the AAV expression cassette comprises a codon-optimized nucleic acid sequence having at least about 80% (for example, at least about 85%, about 90%, about 95%, about 96%, about 97%, about 98%, about 99%, about 99.5%, or greater, such as 100%, including all values and subranges that lie therebetween) identity to the sequence of SEQ ID NO: 8. In some embodiments, the AAV expression cassette comprises or consists of the nucleic acid sequence of SEQ ID NO: 8.

Recombinant Adeno-Associated Virus (rAAV) for Treating Galactosemia

The present disclosure also provides plasmids, comprising any one of the nucleic acid molecules disclosed herein, and cells comprising any one of the nucleic acid molecules or plasmids disclosed herein.

In some embodiments, a plasmid comprises a nucleic acid sequence having at least about 80% (for example, at least about 85%, about 90%, about 95%, about 96%, about 97%, about 98%, about 99%, about 99.5%, or greater, such as 100%, including all values and subranges that lie therebetween) identity to the sequence of SEQ ID NO: 13.

The present disclosure further provides methods of producing a recombinant adeno-associated virus (rAAV). In some embodiments, a method of producing an rAAV comprises contacting an AAV producer cell with any one of the nucleic acid molecules or plasmids disclosed herein. Accordingly, the present disclosure also provides recombinant adeno-associated viruses (rAAVs) produced by the methods of producing rAAVs disclosed herein. In some embodiments, the rAAV comprises an AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAV type rh32.33, AAV type rh8, AAV type rh10, AAV type rh74, AAV type hu.68, avian AAV, bovine AAV, canine AAV, equine AAV, ovine AAV, snake AAV, bearded dragon AAV, AAV218, AAV2g9, AAV-LK03, AAV7m8, AAV Anc80, or AAV PHP.B capsid protein.

In some embodiments, the rAAV comprises an AAV9 capsid protein, an AAV8 capsid protein, and/or an AAVrh10 capsid protein. In some embodiments, the rAAV comprises an AAV9 capsid protein. In some embodiments, the rAAV comprises an AAV8 capsid protein. In some embodiments, the rAAV comprises an AAVrh10 capsid protein.

The present disclosure also provides recombinant adeno-associated viruses (rAAVs). In some embodiments, an rAAV comprises an AAV9 capsid protein and any one of the nucleic acid molecules disclosed herein. In some embodiments, an rAAV comprises an AAV9 capsid protein and a nucleic acid molecule, wherein the nucleic acid molecule comprises an AAV expression cassette, wherein the AAV expression cassette comprises, from 5′ to 3″: (i) a 5′ AAV ITR (e.g., a 5′ AAV ITR comprising the nucleic acid sequence of SEQ ID NO: 6, or a sequence having at least 80%, 85%, 90%, 91%, 92%, 93%, 94%, 95%, 96%, 97%, 98%, 99%, or greater identity to SEQ ID NO: 6), (ii) a CMV-IE enhancer (e.g., a CMV-IE enhancer comprising the nucleic acid sequence of SEQ ID NO: 4, or a sequence having at least 80%, 85%, 90%, 91%, 92%, 93%, 94%, 95%, 96%, 97%, 98%, 99%, or greater identity to SEQ ID NO: 4), (iii) a CB6 promoter (e.g., a CB6 promoter comprising the nucleic acid sequence of SEQ ID NO: 2, or a sequence having at least 80%, 85%, 90%, 91%, 92%, 93%, 94%, 95%, 96%, 97%, 98%, 99%, or greater identity to SEQ ID NO: 2), (iv) a Kozak sequence (e.g., a Kozak sequence comprising the nucleic acid sequence of SEQ ID NO: 5, or a sequence having at least 80%, 85%, 90%, 91%, 92%, 93%, 94%, 95%, 96%, 97%, 98%, 99%, or greater identity to SEQ ID NO: 5), (v) a transgene encoding GALT (e.g., a transgene encoding GALT comprising the nucleic acid sequence of SEQ ID NO: 1, or a sequence having at least 80%, 85%, 90%, 91%, 92%, 93%, 94%, 95%, 96%, 97%, 98%, 99%, or greater identity to SEQ ID NO: 1), (vi) a rabbit globin polyadenylation sequence (e.g., a rabbit globin polyadenylation sequence comprising a nucleic acid sequence of SEQ ID NO: 3, or a sequence having at least 80%, 85%, 90%, 91%, 92%, 93%, 94%, 95%, 96%, 97%, 98%, 99%, or greater identity to SEQ ID NO: 3), and (vii) 3′ AAV ITR (e.g., a 3′ AAV ITR comprising the nucleic acid sequence of SEQ ID NO: 7, or a sequence having at least 80%, 85%, 90%, 91%, 92%, 93%, 94%, 95%, 96%, 97%, 98%, 99%, or greater identity to SEQ ID NO: 7). In some embodiments, an rAAV comprises an AAV9 capsid protein; and a nucleic acid molecule, wherein the nucleic acid molecule comprises an AAV expression cassette, wherein the AAV expression cassette comprises the nucleic acid sequence of SEQ ID NO: 8, or a sequence having at least 80%, 85%, 90%, 91%, 92%, 93%, 94%, 95%, 96%, 97%, 98%, 99%, or greater identity to SEQ ID NO: 8.

In some embodiments, an rAAV is a self-complementary AAV. In some embodiments, an rAAV is a single-stranded AAV.

In some embodiments, preparation of rAAV particles involves culturing a host cell that contains a nucleic acid sequence encoding an AAV capsid protein or fragment thereof: a functional rep gene: a recombinant AAV vector composed of AAV inverted terminal repeats (ITRs) and the AAV expression cassette encoding GALT; and sufficient helper functions to permit packaging of the recombinant AAV vector into the AAV capsid proteins. In some embodiments, the components to be cultured in the host cell to package a rAAV vector in an AAV capsid are provided to the host cell in trans. In some embodiments, any one or more of the required components (e.g., recombinant AAV vector, rep sequences, cap sequences, and/or helper functions) are provided by a stable host cell that has been engineered to contain one or more of the required components. In some embodiments, a stable host cell will contain the required component(s) under the control of an inducible promoter or a constitutive promoter. In some embodiments, a selected stable host cell contains selected component(s) under the control of a constitutive promoter and other selected component(s) under the control of one or more inducible promoters. For example, a stable host cell may be generated which is derived from 293 cells (which contain El helper functions under the control of a constitutive promoter), but which contain the rep and/or cap proteins under the control of inducible promoters. The recombinant AAV vector, rep sequences, cap sequences, and helper functions required for producing the rAAVs disclosed herein may be delivered to the packaging host cell using any appropriate genetic element (for example, a vector). Further details on methods of preparing rAAV particles are provided in Sambrook et al., Molecular Cloning: A Laboratory Manual, Cold Spring Harbor Press, Cold Spring Harbor, N.Y.: K. Fisher et al, J. Virol., 70:520-532 (1993) and U.S. Pat. No. 5,478,745, the contents of each of which are herein incorporated in its entirety for all purposes.

In some embodiments, recombinant AAVs are produced using the triple transfection method, as described in U.S. Pat. No. 6,001,650, the contents of which are herein incorporated in its entirety for all purposes. In some embodiments, the recombinant AAVs are produced by transfecting a host cell with a recombinant AAV vector (comprising the AAV expression cassette encoding GALT) to be packaged into AAV particles, an AAV helper function vector, and an accessory function vector. An AAV helper function vector encodes the “AAV helper function” sequences (i.e., rep and cap), which function in trans for productive AAV replication and encapsidation. Non-limiting examples of AAV helper function vectors include pHLP19 and pRep6cap6 vector, described in U.S. Pat. Nos. 6,001,650 and 6,156,303, respectively, the contents of each of which are herein incorporated in its entirety for all purposes. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some embodiments, recombinant AAVs are produced using baculovirus vectors. Baculovirus vectors are used to produce recombinant AAVs in insect cells (e.g., Spodoptera frugiperda (Sf9) cells).

### Pharmaceutical Compositions

The present disclosure further provides pharmaceutical compositions, comprising: (a) any one of the nucleic acid molecules disclosed herein, any one of the plasmids disclosed herein, any one of the cells disclosed herein, or any one the rAAVs disclosed herein; and (b) a pharmaceutically acceptable carrier.

In some embodiments, the compositions disclosed herein comprise at least one pharmaceutically acceptable carrier, excipient, and/or vehicle, for example, solvents, buffers, solutions, dispersion media, coatings, antibacterial agents, antifungal agents, isotonic agents, and absorption delaying agents. In some embodiments, the pharmaceutically acceptable carrier, excipient, and/or vehicle comprises saline, buffered saline, dextrose, water, glycerol, sterile isotonic aqueous buffer, or a combination thereof. In some embodiments, the pharmaceutically acceptable carrier, excipient, and/or vehicle comprises phosphate buffered saline, sterile saline, lactose, sucrose, calcium phosphate, dextran, agar, pectin, peanut oil, sesame oil, pharmaceutical grades of mannitol, lactose, starch, magnesium stearate, sodium saccharine, cellulose, magnesium carbonate, polyol (e.g., glycerol, propylene glycol, and liquid polyethylene glycol, and the like), or a suitable mixture thereof. In some embodiments, the compositions disclosed herein further comprise emulsifying or wetting agents, or pH buffering agents. Such species may be present in small amounts (e.g., less than 10% by weight of the composition, such as less than 5% by weight of the composition, 2% by weight of the composition, 1% by weight of the composition, or less).

In some embodiments, the compositions disclosed herein further comprise one or more other pharmaceutical ingredients, such as one or more preservatives or chemical stabilizers. Examples of preservatives and chemical stabilizers include, but are not limited to, chlorobutanol, potassium sorbate, sorbic acid, sulfur dioxide, propyl gallate, parabens, ethyl vanillin, glycerin, phenol, parachlorophenol, and albumin. In some embodiments, the compositions disclosed herein further comprise antibacterial agents and/or antifungal agents, such as parabens, chlorobutanol, phenol, sorbic acid, and thimerosal: isotonic agents, such as sugars and sodium chloride; and/or agents delaying absorption, such as aluminum monostearate and gelatin.

In some embodiments, the compositions disclosed herein are formulated to reduce aggregation of AAV particles in the composition, particularly where high rAAV concentrations are present (e.g., ˜10¹³ GC/ml or more). Methods for reducing aggregation of rAAVs include addition of surfactants, pH adjustment, and salt concentration adjustment, as further described in Wright, et al., Molecular Therapy (2005) 12, 171-178, the contents of which are incorporated herein by reference in its entirety for all purposes.

In some embodiments, the pharmaceutical compositions are in a form of an injectable solution or dispersion, such as an aqueous solution or dispersion. In some embodiments, the pharmaceutical composition is a sterile powder for the extemporaneous preparation of sterile injectable solutions or dispersions. Dispersions may be prepared in water, glycerol, liquid polyethylene glycols, oils, or any combination thereof. Delivery vehicles such as liposomes, nanocapsules, microparticles, microspheres, lipid particles, vesicles, and the like, may be used for the introduction of the pharmaceutical compositions disclosed herein.

### Methods of Treating Galactosemia

The present disclosure also provides methods of expressing Galactose-1-Phosphate Uridylyltransferase (GALT) in a cell, comprising: contacting any one of the nucleic acid molecules disclosed herein, any one of the plasmids disclosed herein, any one of the rAAVs disclosed herein, or any one of the compositions disclosed herein with the cell, thereby expressing GALT in the cell. In addition, the present disclosure provides methods of reducing the level of galactose-1-phosphate (Gal-1-P) in a cell, comprising: contacting any one of the nucleic acid molecules disclosed herein, any one of the plasmids disclosed herein, any one of the rAAVs disclosed herein, or any one of the compositions disclosed herein with the cell, thereby reducing the level of Gal-1-P in the cell. In some embodiments, the cell is a brain cell, a liver cell, an ovarian cell, a red blood cell (RBC), or any combination thereof.

In some embodiments, the contacting step is performed as a component of a biochemical assay. In some embodiments, the contacting step is performed in a subject in the course of a treatment regimen. In some embodiments, the contacting step is performed in vitro, ex vivo, or in vivo. In some embodiments, the contacting step is performed in vivo in a subject in need thereof. In some embodiments, the contacting step comprises administering a therapeutically effective amount of the nucleic acid molecule, the plasmid, the rAAV, or the composition to the subject (e.g., a subject in need thereof, such as a subject having galactosemia).

The present disclosure further provides methods of treating galactosemia (e.g., classic galactosemia) or a symptom thereof in a subject in need thereof, comprising: administering to the subject a therapeutically effective amount of any one of the nucleic acid molecules disclosed herein, any one of the plasmids disclosed herein, any one of the rAAVs disclosed herein, or any one of the compositions disclosed herein, thereby treating galactosemia or a symptom thereof in the subject. In a related aspect, the present disclosure provides methods of ameliorating, diminishing the severity of, eliminating, and/or delaying the onset of one or more symptoms of galactosemia (e.g., classic galactosemia) in a subject in need thereof, comprising: administering to the subject a therapeutically effective amount of any one of the nucleic acid molecules disclosed herein, any one of the plasmids disclosed herein, any one of the rAAVs disclosed herein, or any one of the compositions disclosed herein, thereby ameliorating, diminishing the severity of, eliminating, and/or delaying the onset of the one or more symptoms of galactosemia in the subject. In some embodiments, the one or more symptoms of galactosemia are liver dysfunction, kidney dysfunction, sepsis, brain edema, pseudotumor cerebri, feeding difficulty, reduction in growth, cataracts, speech disorder, language disorder, dyspraxia, developmental delay, attention deficit, learning delay, neurological impairment, behavioral disorder, emotional disorder, tremors, fertility dysfunction, primary ovarian insufficiency, anxiety, seizures, depression, attention deficit hyperactivity disorder (ADHD), dementia, or any combination thereof.

In some embodiments, the galactosemia is classic type I galactosemia. In some embodiments, the galactosemia is variant Duarte galactosemia. In some embodiments, the galactosemia is associated with, correlated with, or caused by the deletion of a part or whole of the GALT gene. In some embodiments, the galactosemia is associated with, correlated with, or caused by one or more amino acid mutations in one or both alleles of the galactose-1-phosphate uridyl transferase (GALT) gene. In some embodiments, the GALT gene comprises one or more of the following mutations: Q188R, K285N, S135L, and N314D. In some embodiments, the GALT gene comprises the Q188R mutation. Further details of the GALT gene mutations associated with galactosemia (such as, classic type I galactosemia and variant Duarte galactosemia) are provided in, e.g., Tyfield, et al. Hum Mutat. 1999; 13 (6): 417-30, and Mahmood, et al., Gene. 2012 Nov. 10; 509 (2): 291-4, each of which is incorporated herein by reference in its entirety for all purposes.

In some embodiments, the galactosemia is associated with, correlated with, or caused by a decrease in the activity of the galactose-1-phosphate uridyl transferase enzyme. In some embodiments, the subject is a human subject. In some embodiments, the subject is a neonate or an infant. In some embodiments, the subject suffers from galactosemia. In some embodiments, the subject has been diagnosed with galactosemia. In some embodiments, the subject is at a risk of developing at least one symptom of galactosemia. In some embodiments, the subject at risk of developing at least one symptom of galactosemia is a subject who is diagnosed as having elevated levels of galactose in the blood during newborn screening. In some embodiments, the subject at risk for developing at least one symptom of galactosemia is a subject whose one or both parents is a carrier of one or more mutant GALT gene alleles. In some embodiments, the at least one symptom of galactosemia is liver dysfunction, kidney dysfunction, sepsis, brain edema, pseudotumor cerebri, feeding difficulty, reduction in growth, cataracts, speech disorder, language disorder, dyspraxia, developmental delay, attention deficit, learning delay, neurological impairment, behavioral disorder, emotional disorder, tremors, fertility dysfunction, primary ovarian insufficiency, anxiety, seizures, depression, attention deficit hyperactivity disorder (ADHD), dementia, or any combination thereof.

In some embodiments, the subject is diagnosed as having galactosemia by assaying a level of galactose-1-phosphate (Gal-1-P), a level of galactose, a level of total galactose (e.g., galactose plus Gal-1-P), GALT activity, a level of galactitol, or a combination thereof, in one or more biological samples derived from the subject. In some embodiments, a level of galactitol in a urine sample derived from the subject is assessed. In some embodiments, a level of Gal-1-P in a blood sample derived from the subject is assessed. In some embodiments, a level of total galactose in a blood sample derived from the subject is assessed. In some embodiments, a biological sample (e.g., blood sample) derived from the subject is subjected to a semiquantitative assessment of GALT activity (e.g., a fluorometric Beutler-based method applied to dried blood spots). In some embodiments, assessment of GALT activity comprises performance of an assay based on UDP-glucose consumption, an assay based on UDP-galactose production, a radioenzymatic assay, a liquid chromatography-mass spectrometry/mass spectrometry (LC-MS/MS) assay, or a combination thereof. In some embodiments, a biological sample derived from the subject is assessed for the presence of any one or more of the GALT genetic mutations described herein. For instance, in some embodiments, a biological sample derived from the subject is assessed for the presence of one or more of the following mutations in the GALT gene: −119_−116delGTCA, D98N, S135L, T138M, M142K, F17IS, Q188R, L195P, Y209C, K285N, N314D, Q344K, c.253-2A>G, and 5 kilobyte (kb) deletion. In some embodiments, a biological sample derived from the subject is assessed for a level of Gal-1-P, a level of galactose, a level of total galactose (e.g., galactose plus Gal-1-P), GALT activity, a level of galactitol, a GALT genetic mutation, or a combination thereof during or after a new born screening process.

In some embodiments, the level of galactose in a biological sample (e.g., blood sample) derived from the subject (e.g., subject having galactosemia) is higher than the level of galactose in a biological sample derived from a control healthy subject who does not have galactosemia. In some embodiments, the level of galactose in a biological sample derived from the subject (e.g., subject having galactosemia) at least about 1.1 fold (for example, at least about 1.2 fold, about 1.3 fold, about 1.4 fold, about 1.5 fold, about 1.6 fold, about 1.7 fold, about 1.8 fold, about 1.9 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, or greater, including all values and subranges that lie therebetween) higher than the level of galactose in a biological sample derived from a healthy subject who does not have galactosemia.

In some embodiments, the level of galactose in a biological sample (e.g., blood sample) derived from the subject (e.g., subject having galactosemia) is at least about 2% (for example, at least about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or greater, such as 100%, including all values and subranges that lie therebetween) higher than the level of galactose in a biological sample derived from a healthy subject who does not have galactosemia.

In some embodiments, the level of total galactose (e.g., galactose plus Gal-1-P) in a biological sample (e.g., blood sample) derived from the subject (e.g., subject having galactosemia) is greater than about 4 milligrams per deciliter (mg/dL), such as great than about 5 mg/dL, 6 mg/dL, 7 mg/dL, 8 mg/dL, 9 mg/dL, 10 mg/dL, 12 mg/dL, 14 mg/dL, 16 mg/dL, 18 mg/dL, 20 mg/dL, 22 mg/dL, 24 mg/dL, 26 mg/dL, 28 mg/dL, 30 mg/dL, 32 mg/dL, 34 mg/dL, 36 mg/dL, 38 mg/dL, 40 mg/dL, or higher, including all values and subranges that lie therebetween. In some embodiments, the level of total galactose in a biological sample derived from the subject (e.g., subject having galactosemia) is greater than about 6 mg/dL, such as greater than about 10 mg/dL, such as greater than about 15 mg/dL. Further details regarding levels of total galactose (e.g., galactose plus Gal-1-P) are provided in Pasquali, et al. Laboratory diagnosis of galactosemia: a technical standard and guideline of the American College of Medical Genetics and Genomics (ACMG). Genet Med 20, 3-11 (2018), and Pyhtila, et al. “Newborn screening for galactosemia in the United States: looking back, looking around, and looking ahead.” JIMD Reports vol. 15 (2015): 79-93, the contents of each of which are herein incorporated by reference in its entirety for all purposes.

In some embodiments, the level of Gal-1-P in a biological sample (e.g., blood sample) derived from the subject (e.g., subject having galactosemia) is higher than the level of galactose in a biological sample derived from a control healthy subject who does not have galactosemia (e.g., classic galactosemia). In some embodiments, the level of Gal-1-P in a biological sample derived from the subject (e.g., subject having galactosemia) at least about 1.1 fold (for example, at least about 1.2 fold, about 1.3 fold, about 1.4 fold, about 1.5 fold, about 1.6 fold, about 1.7 fold, about 1.8 fold, about 1.9 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, or greater, including all values and subranges that lie therebetween) higher than the level of Gal-1-P in a biological sample derived from a healthy subject who does not have galactosemia.

In some embodiments, the level of total Gal-1-P in a biological sample derived from the subject (e.g., subject having galactosemia) is at least about 2% (for example, at least about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or greater, such as 100%, including all values and subranges that lie therebetween) higher than the level of galactose in a biological sample derived from a healthy subject who does not have galactosemia.

In some embodiments, the level of Gal-1-P in a biological sample (e.g., blood sample) derived from the subject (e.g., subject having galactosemia) is greater than about 0.9 milligrams per deciliter (mg/dL), such as great than about 1 mg/dL, 2 mg/dL, 3 mg/dL, 4 mg/dL, 5 mg/dL, 6 mg/dL. 7 mg/dL. 8 mg/dL, 9) mg/dL, 10 mg/dL, 12 mg/dL, 14 mg/dL, 16 mg/dL, 18 mg/dL. 20 mg/dL. 22 mg/dL. 24 mg/dL. 26 mg/dL, 28 mg/dL. 30 mg/dL. 32 mg/dL. 34 mg/dl, 36 mg/dL. 38 mg/dL. 40 mg/dL, or higher, including all values and subranges that lie therebetween. In some embodiments, the level of Gal-1-P in a biological sample derived from the subject is greater than about 30 mg/dL, such as greater than about 35 mg/dL, about 40 mg/dL, about 45 mg/dL, about 50 mg/dL, about 55 mg/dL, about 60 mg/dL, about 65 mg/dL, about 70 mg/dL, about 75 mg/dL, about 80 mg/dL, about 85 mg/dL, about 90 mg/dL, about 95 mg/dL., about 100 mg/dL, or higher, including all values and subranges that lie therebetween.

In some embodiments, the enzymatic activity of GALT in a biological sample (e.g., blood sample) derived from the subject (e.g., subject having galactosemia) is less than about 10 enzymatic units per gram of hemoglobin (U/Hb), such as less than about 5 U/Hb, 4 U/Hb. 3 U/Hb, 2 U/Hb, 1 U/Hb, 0.5 U/Hb. 0.2 U/Hb, or lower, including all values and subranges that lie therebetween. In some embodiments, the enzymatic activity of GALT in a biological sample (e.g., blood sample) derived from the subject (e.g., subject having galactosemia) is less than about 10 enzymatic units per deciliter (U/dL), such as less than about 5 U/dL, 4 U/dL, 3 U/dL. 2 U/dL, 1 U/dL, 0.5 U/dL, 0.2 U/dL, or lower, including all values and subranges that lie therebetween. In some embodiments, the enzymatic activity of GALT in a biological sample (e.g., blood sample) derived from the subject (e.g., subject having galactosemia) is less than about 60 micromolar (μM) nicotinamide adenine dinucleotide phosphate (NADPH) when measured using a coupled assay that produces NADPH such that the concentration of NADPH produced in a given period of time corresponds to a particular level of GALT activity in the biological sample. For example, in some embodiments, the enzymatic activity of GALT in a biological sample (e.g., blood sample) derived from the subject (e.g., subject having galactosemia) is less than about 50 μM NADPH, 40 μM NADPH, or lower. In some embodiments, the activity of GALT in a biological sample (e.g., blood sample) derived from the subject is less than about 30 nanomole per hour per milligram (nmol/h/mg) of hemoglobin, such as less than about 25 nmol/h/mg, about 20 nmol/h/mg, about 15 nmol/h/mg, about 10 nmol/h/mg, about 5 nmol/h/mg, about 1 nmol/h/mg, or less, including all values and subranges that lie therebetween. In some embodiments, the activity of GALT in the subject (e.g., subject having galactosemia) is less than about 24.5 nmol/h/mg of hemoglobin. Further details regarding enzymatic activity of GALT are provided in Pasquali, et al. Laboratory diagnosis of galactosemia: a technical standard and guideline of the American College of Medical Genetics and Genomics (ACMG). Genet Med 20, 3-11 (2018), Pyhtila, et al. “Newborn screening for galactosemia in the United States: looking back, looking around, and looking ahead.” JIMD Reports vol. 15 (2015): 79-93, and Mayo Clinic guidelines at mayocliniclabs.com/test-catalog/Clinical+and+Interpretive/84360, the contents of each of which are herein incorporated by reference in its entirety for all purposes.

In some embodiments, the enzymatic activity of GALT in a biological sample (e.g., blood sample) derived from the subject (e.g., subject having galactosemia) is less than about 80% of that in a biological sample (e.g., blood sample) derived from a control healthy subject who does not have galactosemia (e.g., classic galactosemia), such as less than about 70%, 60%, 50%, 40%, 30%, 20%, 15%, 12%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or lower. In some embodiments, the enzymatic activity of GALT in a biological sample (e.g., blood sample) derived from the subject (e.g., subject having galactosemia) is less than about 5% of that in a biological sample (e.g., blood sample) derived from a control healthy subject who does not have galactosemia (e.g., classic galactosemia).

Accordingly, the present disclosure provides methods of treating galactosemia (e.g., classic galactosemia) or a symptom thereof in a subject in need thereof (e.g., a subject having galactosemia), comprising: (i) determining the activity of GALT in a biological sample (e.g., blood sample) obtained from the subject; and, (ii) if the activity of GALT determined in step (i) is less than about (1) 24.5 nmol/h/mg of hemoglobin, (2) 3 U/Hb, (3) 3 U/dL, (4) 60 μM NADPH, and/or 5% of that in a biological sample (e.g., blood sample) derived from a control healthy subject who does not have galactosemia (e.g., classic galactosemia), administering to the subject a therapeutically effective amount of any one of the nucleic acid molecules disclosed herein, any one of the plasmids disclosed herein, any one of the rAAVs disclosed herein, or any one of the compositions disclosed herein, thereby treating galactosemia in the subject. In some embodiments, the activity of GALT is less than about 24.5 nmol/h/mg of hemoglobin. In some embodiments, the activity of GALT is less than about 3 U/Hb. In some embodiments, the activity of GALT is less than about 3 U/dL. In some embodiments, the activity of GALT is less than about 60 μM NADPH. In some embodiments, the activity of GALT is less than about 5% of that in a biological sample (e.g., blood sample) derived from a control healthy subject who does not have galactosemia (e.g., classic galactosemia), such as less than about 1%. In some embodiments, the subject is an infant. In some embodiments, the biological sample is a blood sample. In some embodiments, the subject has classic galactosemia. In some embodiments, the symptom of galactosemia is liver dysfunction, kidney dysfunction, sepsis, brain edema, pseudotumor cerebri, feeding difficulty, reduction in growth, cataracts, speech disorder, language disorder, dyspraxia, developmental delay, attention deficit, learning delay, neurological impairment, behavioral disorder, emotional disorder, tremors, fertility dysfunction, primary ovarian insufficiency, anxiety, seizures, depression, attention deficit hyperactivity disorder (ADHD), dementia, or any combination thereof.

Relatedly, the present disclosure provides methods of ameliorating, diminishing the severity of, eliminating, and/or delaying the onset of one or more symptoms of galactosemia (e.g., classic galactosemia) in a subject in need thereof, comprising: (i) determining the activity of GALT in a biological sample (e.g., blood sample) obtained from the subject; and if the activity of GALT determined in step (i) is less than about (1) 24.5 nmol/h/mg of hemoglobin, (2) 3 U/Hb, (3) 3 U/dL, (4) 60 μM NADPH, and/or 5% of that in a biological sample (e.g., blood sample) derived from a control healthy subject who does not have galactosemia (e.g., classic galactosemia), (ii) administering to the subject a therapeutically effective amount of any one of the nucleic acid molecules disclosed herein, any one of the plasmids disclosed herein, any one of the rAAVs disclosed herein, or any one of the compositions disclosed herein, thereby ameliorating, diminishing the severity of, eliminating, and/or delaying the onset of the one or more symptoms of galactosemia in the subject. In some embodiments, the activity of GALT is less than about 24.5 nmol/h/mg of hemoglobin. In some embodiments, the activity of GALT is less than about 3 U/Hb. In some embodiments, the activity of GALT is less than about 3 U/dL. In some embodiments, the activity of GALT is less than about 60 μM NADPH. In some embodiments, the activity of GALT is less than about 5% of that in a biological sample (e.g., blood sample) derived from a control healthy subject who does not have galactosemia (e.g., classic galactosemia), such as less than about 1%. In some embodiments, the subject is an infant. In some embodiments, the biological sample is a blood sample. In some embodiments, the one or more symptoms of galactosemia are liver dysfunction, kidney dysfunction, sepsis, brain edema, pseudotumor cerebri, feeding difficulty, reduction in growth, cataracts, speech disorder, language disorder, dyspraxia, developmental delay, attention deficit, learning delay, neurological impairment, behavioral disorder, emotional disorder, tremors, fertility dysfunction, primary ovarian insufficiency, anxiety, seizures, depression, attention deficit hyperactivity disorder (ADHD), dementia, or any combination thereof.

In some embodiments of the preceding aspects, the level of Gal-1-P in a biological sample (e.g., blood sample) derived from the subject (e.g., subject having galactosemia) is more than that in a healthy subject who does not have galactosemia (e.g., classic galactosemia). In some embodiments, the level of Gal-1-P in a biological sample derived from the subject (e.g., subject having galactosemia) is at least about 1.1 fold (for example, at least about 1.2 fold, about 1.3 fold, about 1.4 fold, about 1.5 fold, about 1.6 fold, about 1.7 fold, about 1.8 fold, about 1.9 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, or greater, including all values and subranges that lie therebetween) higher than the level of Gal-1-P in a biological sample derived in a healthy subject who does not have galactosemia (e.g., classic galactosemia).

In some embodiments of the preceding aspects, the level of Gal-1-P in a biological sample (e.g., blood sample) derived from the subject (e.g., subject having galactosemia) is at least about 2% (for example, at least about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or higher, such as 100%, including all values and subranges that lie therebetween) higher than the level of Gal-1-P in a biological sample derived in a healthy subject who does not have galactosemia (e.g., classic galactosemia).

In some embodiments of the preceding aspects, the level of Gal-1-P in a biological sample (e.g., blood sample) derived from the subject (e.g., subject having galactosemia) is greater than about 0.9 milligrams per deciliter (mg/dL), such as great than about 1 mg/dl, 2 mg/dL, 3 mg/dL, 4 mg/dL, 5 mg/dL, 6 mg/dL, 7 mg/dL, 8 mg/dL, 9 mg/dL, 10 mg/dL, 12 mg/dL, 14 mg/dL, 16 mg/dL, 18 mg/dL, 20 mg/dL, 22 mg/dL, 24 mg/dL, 26 mg/dL, 28 mg/dL, 30 mg/dL, 32 mg/dL, 34 mg/dL, 36 mg/dL, 38 mg/dL, 40 mg/dL, or higher, including all values and subranges that lie therebetween. In some embodiments, the level of Gal-1-P in a biological sample derived from the subject (e.g., subject having galactosemia) is greater than about 30 mg/dL, such as greater than about 35 mg/dL, about 40 mg/dL, about 45 mg/dL, about 50 mg/dL, about 55 mg/dL, about 60 mg/dL, about 65 mg/dL, about 70 mg/dL, about 75 mg/dL, about 80 mg/dL, about 85 mg/dL, about 90 mg/dL, about 95 mg/dL, about 100 mg/dL, or higher, including all values and subranges that lie therebetween.

In some embodiments of the preceding aspects, the level of total galactose in a biological sample (e.g., blood sample) derived from the subject (e.g., subject having galactosemia) is greater than about 6 milligrams per deciliter (mg/dL), such as great than about 7 mg/dL, 8 mg/dL, 9 mg/dL, 10 mg/dL, 12 mg/dL, 14 mg/dL, 16 mg/dL, 18 mg/dL, 20 mg/dL, 22 mg/dL, 24 mg/dL, 26 mg/dL, 28 mg/dL, 30 mg/dL, 32 mg/dL, 34 mg/dL, 36 mg/dL, 38 mg/dL, 40 mg/dL, or higher, including all values and subranges that lie therebetween. In some embodiments, the level of total galactose in a biological sample derived from the subject (e.g., subject having galactosemia) is greater than about 10 mg/dL, such as greater than about 15 mg/dL.

In some embodiments, the enzymatic activity of GALT in a biological sample (e.g., blood sample) derived from the subject (e.g., subject having galactosemia) is less than about 80% of that in a biological sample (e.g., blood sample) derived from a control healthy subject who does not have galactosemia (e.g., classic galactosemia), such as less than about 70%, 60%, 50%, 40%, 30%, 20%, 15%, 12%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or lower. In some embodiments, the enzymatic activity of GALT in a biological sample (e.g., blood sample) derived from the subject (e.g., subject having galactosemia) is less than about 5% of that in a biological sample (e.g., blood sample) derived from a control healthy subject who does not have galactosemia (e.g., classic galactosemia).

The present disclosure also provides methods of treating galactosemia (e.g., classic galactosemia) or a symptom thereof in a subject in need thereof, comprising: (i) determining the level of Gal-1-P in a biological sample (e.g., blood sample) derived from the subject; and (ii) if the level of Gal-1-P determined in step (i) is greater than about 0.9 mg/dL (such as greater than about 1 mg/dL, 2 mg/dL, 3 mg/dL, 4 mg/dL, 5 mg/dL, 6 mg/dL, 7 mg/dl, 8 mg/dl, 9 mg/dL, 10 mg/dL, 12 mg/dL, 14 mg/dL, 16 mg/dL, 18 mg/dL, 20 mg/dL, 22 mg/dL, 24 mg/dL, 26 mg/dL. 28 mg/dL, 30 mg/dL, 32 mg/dL, 34 mg/dL, 36 mg/dL, 38 mg/dL, 40 mg/dL, or higher), administering to the subject a therapeutically effective amount of any one of the nucleic acid molecules disclosed herein, any one of the plasmids disclosed herein, any one of the rAAVs disclosed herein, or any one of the compositions disclosed herein, thereby treating galactosemia in the subject. In some embodiments, the biological sample is a blood sample (e.g., a sample including red blood cells). In some embodiments, the Gal-1-P level is at least about 30 mg/dL. In some embodiments, the subject is an infant. In some embodiments, the symptom of galactosemia is liver dysfunction, kidney dysfunction, sepsis, brain edema, pseudotumor cerebri, feeding difficulty, reduction in growth, cataracts, speech disorder, language disorder, dyspraxia, developmental delay, attention deficit, learning delay, neurological impairment, behavioral disorder, emotional disorder, tremors, fertility dysfunction, primary ovarian insufficiency, anxiety, seizures, depression, attention deficit hyperactivity disorder (ADHD), dementia, or any combination thereof.

Relatedly, the present disclosure provides methods of treating galactosemia (e.g., classic galactosemia) or a symptom thereof in a subject in need thereof, comprising: (i) determining the level of total galactose (e.g., galactose plus Gal-1-P) in a biological sample (e.g., blood sample) derived from the subject; and if the level of total galactose determined in step (i) is greater than about 6 mg/dL (such as greater than about 7 mg/dL, 8 mg/dL, 9 mg/dL, 10 mg/dL, 12 mg/dL, 14 mg/dL, 16 mg/dL, 18 mg/dL, 20 mg/dL, 22 mg/dL, 24 mg/dL, 26 mg/dL, 28 mg/dL, 30 mg/dL, 32 mg/dL, 34 mg/dL, 36 mg/dL, 38 mg/dL, 40 mg/dL, or higher), (ii) administering to the subject a therapeutically effective amount of any one of the nucleic acid molecules disclosed herein, any one of the plasmids disclosed herein, any one of the rAAVs disclosed herein, or any one of the compositions disclosed herein, thereby treating galactosemia in the subject. In some embodiments, the biological sample is a blood sample (e.g., a sample including red blood cells). In some embodiments, the total galactose level is at least about 10 mg/dL, such as at least about 15 mg/dL. In some embodiments, the subject is an infant. In some embodiments, the symptom of galactosemia is liver dysfunction, kidney dysfunction, sepsis, brain edema, pseudotumor cerebri, feeding difficulty, reduction in growth, cataracts, speech disorder, language disorder, dyspraxia, developmental delay, attention deficit, learning delay, neurological impairment, behavioral disorder, emotional disorder, tremors, fertility dysfunction, primary ovarian insufficiency, anxiety, seizures, depression, attention deficit hyperactivity disorder (ADHD), dementia, or any combination thereof.

The present disclosure also provides methods of ameliorating, diminishing the severity of, eliminating, and/or delaying the onset of one or more symptoms of galactosemia (e.g., classic galactosemia) in a subject in need thereof, comprising: (i) determining the level of Gal-1-P in a biological sample (e.g., blood sample) derived from the subject; and if the level of Gal-1-P determined in step (i) is greater than about 0.9 mg/dL (such as greater than about 1 mg/dL, 2 mg/dL, 3 mg/dL, 4 mg/dL, 5 mg/dL, 6 mg/dL, 7 mg/dL, 8 mg/dL, 9 mg/dL, 10 mg/dL, 12 mg/dL, 14 mg/dL, 16 mg/dL, 18 mg/dL, 20 mg/dL, 22 mg/dL, 24 mg/dL, 26 mg/dL, 28 mg/dL, 30 mg/dL, 32 mg/dL, 34 mg/dL, 36 mg/dL, 38 mg/dL, 40 mg/dL, or higher), (ii) administering to the subject a therapeutically effective amount of any one of the nucleic acid molecules disclosed herein, any one of the plasmids disclosed herein, any one of the rAAVs disclosed herein, or any one of the compositions disclosed herein, thereby ameliorating, diminishing the severity of, eliminating, and/or delaying the onset of the one or more symptoms of galactosemia in the subject. In some embodiments, the biological sample is a blood sample (e.g., a sample including red blood cells). In some embodiments, the Gal-1-P level is at least about 30 mg/dL. In some embodiments, the subject is an infant. In some embodiments, the one or more symptoms of galactosemia are liver dysfunction, kidney dysfunction, sepsis, brain edema, pseudotumor cerebri, feeding difficulty, reduction in growth, cataracts, speech disorder, language disorder, dyspraxia, developmental delay, attention deficit, learning delay, neurological impairment, behavioral disorder, emotional disorder, tremors, fertility dysfunction, primary ovarian insufficiency, anxiety, seizures, depression, attention deficit hyperactivity disorder (ADHD), dementia, or any combination thereof.

Relatedly, the present disclosure provides methods of ameliorating, diminishing the severity of, eliminating, and/or delaying the onset of one or more symptoms of galactosemia (e.g., classic galactosemia) in a subject in need thereof, comprising: (i) determining the level of total galactose (e.g., galactose plus Gal-1-P) in a biological sample (e.g., blood sample) derived from the subject; and if the level of galactose determined in step (i) is greater than about 6 mg/dL (such as greater than about 7 mg/dL, 8 mg/dL, 9 mg/dL, 10 mg/dL, 12 mg/dL, 14 mg/dL, 16 mg/dL, 18 mg/dL, 20 mg/dL, 22 mg/dL, 24 mg/dL, 26 mg/dL, 28 mg/dL, 30 mg/dL, 32 mg/dL, 34 mg/dL, 36 mg/dL, 38 mg/dL, 40 mg/dL, or higher), (ii) administering to the subject a therapeutically effective amount of any one of the nucleic acid molecules disclosed herein, any one of the plasmids disclosed herein, any one of the rAAVs disclosed herein, or any one of the compositions disclosed herein, thereby ameliorating, diminishing the severity of, eliminating, and/or delaying the onset of the one or more symptoms of galactosemia in the subject. In some embodiments, the biological sample is a blood sample (e.g., a sample including red blood cells). In some embodiments, the total galactose level is at least about 10 mg/dL, such as at least about 15 mg/dL. In some embodiments, the subject is an infant. In some embodiments, the one or more symptoms of galactosemia are liver dysfunction, kidney dysfunction, sepsis, brain edema, pseudotumor cerebri, feeding difficulty, reduction in growth, cataracts, speech disorder, language disorder, dyspraxia, developmental delay, attention deficit, learning delay, neurological impairment, behavioral disorder, emotional disorder, tremors, fertility dysfunction, primary ovarian insufficiency, anxiety, seizures, depression, attention deficit hyperactivity disorder (ADHD), dementia, or any combination thereof.

In some embodiments of the preceding aspects, the level or activity of GALT in a biological sample (e.g., blood sample) derived from the subject is less than the level or activity, respectively, of GALT in a healthy subject who does not have galactosemia (e.g., classic galactosemia). In some embodiments, the level or activity of the GALT in the subject is less than about 0.1%, less than about 0.2%, less than about 0.3%, less than about 0.4%, less than about 0.5%, less than about 0.6%, less than about 0.7%, less than about 0.8%, less than about 0.9%, less than about 1%, less than about 2%, less than about 3%, less than about 4%, less than about 5%, less than about 10%, less than about 15%, less than about 20%, less than about 25%, less than about 30%, less than about 40%, less than about 50%, less than about 60%, less than about 70%, less than about 80%, less than about 90%, or less than about 95% of the level or activity, respectively, of GALT in a biological sample (e.g., blood sample) derived from a healthy subject who does not have galactosemia (e.g., classic galactosemia). In some embodiments, the enzyme activity of GALT is assayed using Liquid Chromatography-Tandem Mass Spectrometry (LC-MS/MS).

In some embodiments of the preceding aspects, the activity of GALT in the subject is less than about 30 nanomole per hour per milligram (nmol/h/mg) of hemoglobin, such as less than about 25 nmol/h/mg, about 20 nmol/h/mg, about 15 nmol/h/mg, about 10 nmol/h/mg, about 5 nmol/h/mg, about 1 nmol/h/mg, or less, including all values and subranges that lie therebetween. In some embodiments, activity of GALT in the subject is less than about 24.5 nmol/h/mg of hemoglobin. In some embodiments, the enzymatic activity of GALT in a biological sample (e.g., blood sample) derived from the subject is less than about 10 enzymatic units per gram of hemoglobin (U/Hb), such as less than about 5 U/Hb, 4 U/Hb, 3 U/Hb, 2 U/Hb, 1 U/Hb, 0.5 U/Hb, 0.2 U/Hb, or lower, including all values and subranges that lie therebetween. In some embodiments, the enzymatic activity of GALT in a biological sample (e.g., blood sample) derived from the subject is less than about 10 enzymatic units per deciliter (U/dL), such as less than about 5 U/dL, 4 U/dL, 3 U/dL 2 U/dL, 1 U/dL, 0.5 U/dL, 0.2 U/dL, or lower, including all values and subranges that lie therebetween. In some embodiments, the enzymatic activity of GALT in a biological sample (e.g., blood sample) derived from the subject is less than about 60 micromolar (μM) nicotinamide adenine dinucleotide phosphate (NADPH) when measured using a coupled assay that produces NADPH such that the concentration of NADPH produced in a given period of time corresponds to a particular level of GALT activity in the biological sample. For example, in some embodiments, the enzymatic activity of GALT in a biological sample (e.g., blood sample) derived from the subject is less than about 50 μM NADPH, 40 μM NADPH, or lower.

In some embodiments of any of the preceding aspects, the level of total galactose in a biological sample (e.g., blood sample) derived from the subject after administration is lower as compared to (i) the level of total galactose (e.g., galactose plus Gal-1-P) in a control subject having galactosemia (e.g., classic galactosemia) who is not administered the nucleic acid molecule, the plasmid, the cell, the rAAV or the composition, or (ii) the level of total galactose in the subject before administration. In some embodiments, the level of total galactose in a biological sample derived from the subject after administration is at least about 2% (for example, at least about 3%, about 4%, about 5%, about 10%, about 15%, about 20%, about 25%, about 30%, about 35%, about 40) %, about 45%, about 50%, about 55%, about 60%, about 65%, about 70%, about 75%, about 80%, about 85%, about 90%, about 95%, or higher, such as 100%, including all values and subranges that lie therebetween) lower as compared to (i) the level of total galactose in a control subject having galactosemia who is not administered the nucleic acid molecule, the plasmid, the cell, the rAAV or the composition, or (ii) the level of total galactose in the subject before administration.

In some embodiments of any of the preceding aspects, the level of Gal-1-P in a biological sample (e.g., blood sample) derived from the subject after administration is lower as compared to (i) the level of Gal-1-P in a control subject having galactosemia (e.g., classic galactosemia) who is not administered the nucleic acid molecule, the plasmid, the cell, the rAAV or the composition, or (ii) the level of Gal-1-P in the subject before administration. In some embodiments, the level of Gal-1-P in a biological sample derived from the subject after administration is at least about 2% (for example, at least about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or higher, such as 100%, including all values and subranges that lie therebetween) lower as compared to (i) the level of Gal-1-P in a control subject having galactosemia who is not administered the nucleic acid molecule, the plasmid, the cell, the rAAV or the composition, or (ii) the level of Gal-1-P in the subject before administration. In some embodiments, the level of Gal-1-P in a biological sample derived from the subject after administration is lower than about 30 mg/dL, such as lower than about 20 mg/dL, 10 mg/dL, 5 mg/dL, 1 mg/dL, 0.9 mg/dL, 0.8 mg/dL, 0.7 mg/dL, 0.6 mg/dL, 0.5 mg/dL, 0.4 mg/dL, 0.3 mg/dL. 0.2 mg/dL, 0.1 mg/dL, 0.05 mg/dL, 0.01 mg/dL, 0.005 mg/dL, 0.001 mg/dL, or less. In some embodiments, the level of Gal-1-P in a biological sample derived from the subject after administration is lower than about 1 mg/dL.

In some embodiments of any of the preceding aspects, the level of GALT in a biological sample (e.g., blood sample) derived from the subject after administration is higher as compared to (i) the level of GALT in a control subject having galactosemia (e.g., classic galactosemia) who is not administered the nucleic acid molecule, the plasmid, the cell, the rAAV, or the composition, or (ii) the level of GALT in the subject before administration. In some embodiments, the level of GALT in a biological sample derived from the subject is at least about 2% (for example, at least about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, 100%, about 200%, about 300%, about 400%, about 500%, about 600%, about 700%, about 800%, about 900% or about 1000%, or greater, including all values and subranges that lie therebetween) higher after administration as compared to (i) the level of GALT in a control subject having galactosemia who is not administered the nucleic acid molecule, the plasmid, the cell, the rAAV, or the composition, or (ii) the level of GALT in the subject before administration. In some embodiments, the level of GALT in a biological sample (e.g., blood sample) derived from the subject after administration is more than about 5% (for example, at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, 100%, or greater, including all values and subranges that lie therebetween) of the level of GALT in a biological sample derived from a healthy subject who does not have galactosemia (e.g., classic galactosemia).

In some embodiments of any of the preceding aspects, the activity of GALT in a biological sample (e.g., blood sample) derived from the subject is higher after administration as compared to (i) the activity of GALT in a control subject having galactosemia (e.g., classic galactosemia) who is not administered the nucleic acid molecule, the plasmid, the cell, the rAAV, or the composition, or (ii) the activity of GALT in the subject before administration. In some embodiments, the activity of GALT in a biological sample derived from the subject is at least about 2% (for example, at least about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, 100%, about 200%, about 300%, about 400%, about 500%, about 600%, about 700%, about 800%, about 900% or about 1000%, or greater, including all values and subranges that lie therebetween) higher after administration as compared to (i) the activity of GALT in a control subject having galactosemia who is not administered the nucleic acid molecule, the plasmid, the cell, the rAAV or the composition, or (ii) the activity of GALT in the subject before administration. In some embodiments, the activity of GALT in a biological sample (e.g., blood sample) derived from the subject after administration is more than about 5% (for example, at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or greater such as 100%, including all values and subranges that lie therebetween) of the activity of GALT in a biological sample derived from a healthy subject who does not have galactosemia (e.g., classic galactosemia).

In some embodiments of any of the preceding aspects, the activity of GALT in a biological sample (e.g., blood sample) derived from the subject after administration is higher than 24.5 nmol/h/mg of hemoglobin, for example, at least about 30 nmol/h/mg of hemoglobin, about 35 nmol/h/mg of hemoglobin, about 40 nmol/h/mg of hemoglobin, about 45 nmol/h/mg of hemoglobin, about 50 nmol/h/mg of hemoglobin, about 55 nmol/h/mg of hemoglobin, about 60 nmol/h/mg of hemoglobin, about 65 nmol/h/mg, or higher, of hemoglobin, including all values and subranges that lie therebetween.

In some embodiments of any of the preceding aspects, the level of GALT, the activity of GALT, the level of galactose, and/or the level of galactose-1-phosphate is measured in a brain cell, a liver cell, an ovarian cell, or a red blood cell (RBC) derived from the subject.

In some embodiments of any of the preceding aspects, the administration diminishes the severity of a symptom of galactosemia. In some embodiments, the administration delays or prevents the onset of a symptom of galactosemia. In some embodiments, the administration eliminates a symptom of galactosemia. In some embodiments, the symptom of galactosemia is liver dysfunction, kidney dysfunction, sepsis, brain edema, pseudotumor cerebri, feeding difficulty, reduction in growth, cataracts, speech disorder, language disorder, dyspraxia, developmental delay, attention deficit, learning delay, neurological impairment, behavioral disorder, emotional disorder, tremors, fertility dysfunction, primary ovarian insufficiency, anxiety, seizures, depression, attention deficit hyperactivity disorder (ADHD), dementia, or any combination thereof.

In some embodiments of any of the preceding aspects, the method comprises administering a therapeutically effective amount of an rAAV, wherein the therapeutically effective amount is in a range of about 10⁵ genome copies to 10²⁰ genome copies per kilogram (kg), for example, about 10⁶ genome copies/kg, about 10⁷ genome copies/kg, about 10⁸ genome copies/kg, about 10⁹ genome copies/kg, about 10¹⁰ genome copies/kg, about 10¹¹ genome copies/kg, about 10¹² genome copies/kg, about 10¹³ genome copies/kg, about 10¹⁴ genome copies/kg, about 10¹⁵ genome copies/kg, about 10¹⁶ genome copies/kg, about 10¹⁷ genome copies/kg, about 10¹⁸ genome copies/kg, or about 10¹⁹ genome copies/kg, including all values and subranges that lie therebetween. In some embodiments, the method comprises administering a therapeutically effective amount of rAAV, wherein the therapeutically effective amount is in a range of 10¹⁰ genome copies to 10¹⁴ genome copies per kilogram.

In some embodiments of any of the preceding aspects, the therapeutically effective amount is in the range of about 10⁵ to 10²⁰ genome copies per subject, for example, about 10⁶ genome copies per subject, about 10⁷ genome copies per subject, about 10⁸ genome copies per subject, about 10⁹ genome copies per subject, about 10¹⁰ genome copies per subject, about 10¹¹ genome copies per subject, about 10¹² genome copies per subject, about 10¹³ genome copies per subject, about 10¹⁴ genome copies per subject, about 10¹⁵ genome copies per subject, about 10¹⁶ genome copies per subject, about 10¹⁷ genome copies per subject, about 10¹⁸ genome copies per subject, or about 10¹⁹ genome copies per subject, including all values and subranges that lie therebetween. In some embodiments, the therapeutically effective amount is in the range of about 10⁹ to 10¹⁶ genome copies per subject.

In some embodiments of any of the preceding aspects, the therapeutically effective amount is administered in a volume of about 1 microliter (μl) to about 100 milliliter (mL) of solution, for example, about 10 μl, about 50 μl, about 100 μl, about 500 μl, about 1 mL, about 20 mL, about 30 mL, about 40 mL, about 50 mL, about 60 mL, about 70 mL, about 80 mL, about 90 mL, or about 100 mL, including all values and subranges that lie therebetween. The volume used may depend on the dose of the rAAV, and the route of administration. For example, for intrathecal or intracerebral administration a volume in the range of about 1 μl to about 10 μl, or about 10 μl to about 100 μl may be used. For intravenous administration a volume in range of about 10 μl to about 100 μl, or about 100 μl to 1 mL, or about 1 mL to about 10 mL, or more may be used.

In some embodiments of any of the preceding aspects, more than one administration (e.g., two, three, four or more administrations) may be employed to achieve the desired level of gene expression over a period of various intervals, e.g., daily, weekly, monthly, yearly, etc.

In some embodiments of any of the preceding aspects, the administration is by injection into the central nervous system. Other modes of administration that may be used include oral, rectal, transmucosal, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular [including administration to skeletal, diaphragm, and/or cardiac muscle], intradermal, intrapleural, intracerebral, and intraarticular), topical (e.g., to skin and/or mucosal surfaces, including airway surfaces, and transdermal administration), intralymphatic, and the like, as well as direct tissue or organ injection (e.g., to liver, skeletal muscle, cardiac muscle, diaphragm muscle or brain).

In some embodiments of any of the preceding aspects, the nucleic acid molecule, the plasmid, the cell, the rAAV, or the composition is administered into the bloodstream of the subject. Administration into the bloodstream may be by injection into a vein, an artery, or any other vascular conduit. In some embodiments, the nucleic acid molecule, the plasmid, the cell, the rAAV, or the composition is administered intravascularly. In some embodiments, the nucleic acid molecule, the plasmid, the cell, the rAAV, or the composition is administered intravenously.

In some embodiments of any of the preceding aspects, the nucleic acid molecule, the plasmid, the cell, the rAAV, or the composition is delivered to brain tissue, meninges, neuronal cells, glial cells, astrocytes, oligodendrocytes, cerebrospinal fluid (CSF), interstitial spaces, and the like. In some embodiments, recombinant AAVs are delivered directly to the spinal cord or brain by injection into the ventricular region, as well as to the striatum (e.g., the caudate nucleus or putamen of the striatum), and neuromuscular junction, or cerebellar lobule, with a needle, catheter or related device, using neurosurgical techniques, such as by stereotactic injection.

In some embodiments of any of the preceding aspects, the administration comprises administering the nucleic acid molecule, the plasmid, the cell, the rAAV, or the composition by more than route concurrently or at different time points.

The present disclosure also provides kits comprising one or more agents (e.g., any one of the nucleic acid molecules disclosed herein, any one of the plasmids disclosed herein, any one of the rAAVs disclosed herein, or any one of the compositions disclosed herein). In some embodiments, the kits are pharmaceutical or diagnostic or research kits to be used in therapeutic, diagnostic or research applications. A kit may include one or more containers housing the agents disclosed herein and instructions for use. In certain embodiments, agents in a kit are in a pharmaceutical formulation and dosage suitable for a particular application and for a method of administration of the agents. In some embodiments, the container is a syringe, vial, tube, topical application devices, IV needle tubing and bag, and other containers.

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term's definition in the application, the definition that appears in this application controls. However, mention of any reference, article, publication, patent, patent publication, and patent application cited herein is not, and should not be taken as an acknowledgment, or any form of suggestion, that they constitute valid prior art or form part of the common general knowledge in any country in the world.

Unless the context indicates otherwise, it is specifically intended that the various features described herein can be used in any combination.

### EXAMPLES

### Example 1: Tissue Transduction of scAAV-GALT in GALT Knockout (KO) Mouse Model of Galactosemia

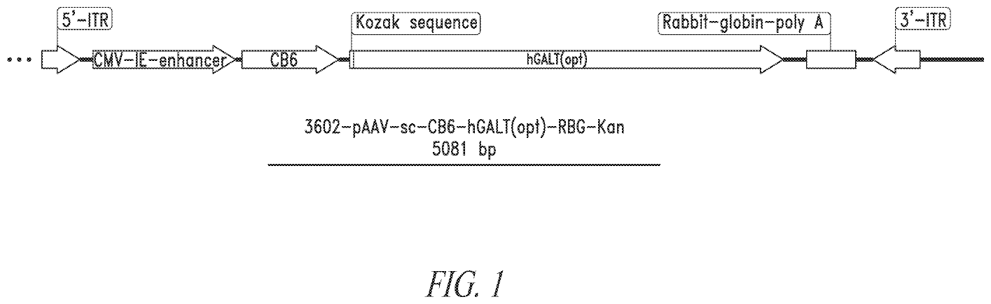

A self-complementary AAV (scAAV) vector was constructed that comprises the AAV expression cassette shown in FIG. 1 (e.g., SEQ ID NO: 8). The AAV expression cassette comprises a CB6 promoter with a CMV-IE enhancer operably linked to the codon-optimized nucleic acid sequence of SEQ ID NO: 1 encoding a GALT protein having the amino acid sequence of SEQ ID NO: 14. The scAAV vector was packaged into scAAV particles using the triple transfection method.

To evaluate effectiveness, the scAAV-GALT particles were administered to a GALT-knockout mouse model of galactosemia, and the mice were examined to determine whether the scAAV particles are able to transduce tissues and express the GALT transgene. The study design is shown below in Table 1.

TABLE 1
AIM 1 A/B Study: AAV9-GALT in knockout (KO) model

| | | | Mice/ | Dose | Necropsy |
|---|---|---|---|---|---|
| Group | Mouse strain | Description | group | (vg/kg) | time points |
| 1 | GALT-KO | Low dose | 10 | 5.00 × 10¹² | 4 and 8 weeks |
| 2 | GALT-KO | Mid dose | 10 | 2.50 × 10¹³ | 4 and 8 weeks |
| 3 | GALT-KO | High dose | 10 | 5.00 × 10¹³ | 4 and 8 weeks |
| 4 | GALT-KO | Uninjected | 10 | NA | 4 and 8 weeks |
| | | control | | | |
| 5 | Wild type | WT control | 5 | 2.50 × 10¹³ | 4 weeks |
| | (WT) | | | | |
| 6 | Wild type | Uninjected | 5 | NA | 4 weeks |
| | (WT) | control | | | |

The GalT gene trapped mouse model (alternately referred to as Galt −/−) is useful for preclinical studies in classic galactosemia. The model is sensitive to galactose challenge at birth, develops a subfertility phenotype reminiscent of primary ovarian insufficiency (POI), and shows signs of motor function defects allowing for multiple disease-relevant phenotypic readouts. Additionally, the model shows consistent elevation in metabolic markers of the disease, such as galactose 1-phosphate (gal-1P).

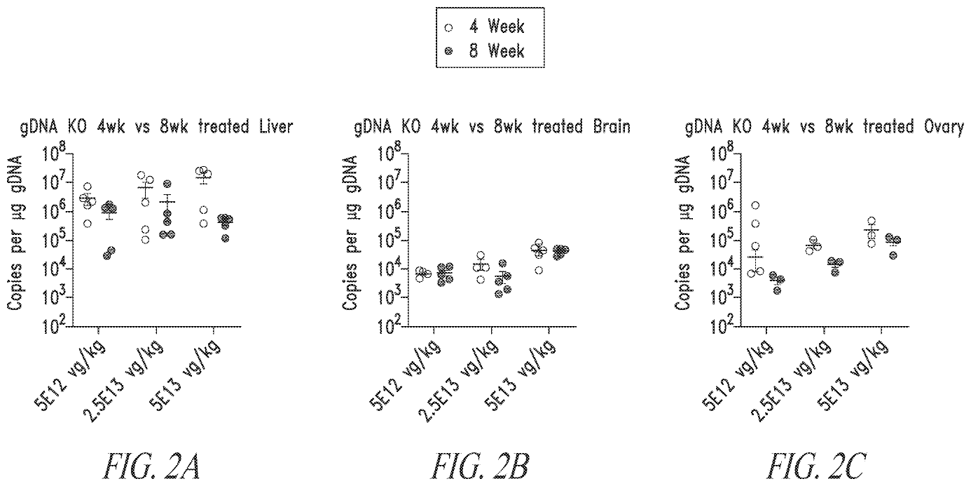

Ten week-old mice were injected intravenously with three different doses of the SCAAV-GALT (5.00×10¹² vector genomes (vg)/kg: 2.50×10¹³ vg/kg: or 5.00×10¹³ vg/kg). Liver tissue, brain tissue, ovarian tissue, and red blood cells (RBCs) were harvested at 4 weeks and 8 weeks post dosing. DNA was extracted from the tissues, and digital polymerase chain reaction (PCR) was used to analyze the vector copy per microgram of total genomic DNA. The results are shown in FIGS. 2A-2C. The results show that the scAAV-GALT is able to transduce into each of the tissues tested. Moreover, the amount of scAAV-GALT in these tissues correlates with the dose administered, and is persistent at least until 8 weeks post dosing.

### Example 2: Expression of GALT mRNA in GALT KO Mouse Model of Galactosemia

Ten week-old mice were injected intravenously with three different doses of the SCAAV-GALT (5.00×10¹² vg/kg: 2.50×10¹³ vg/kg: or 5.00×10¹³ vg/kg). Liver tissue, brain tissue, ovarian tissue, and red blood cells (RBCs) were harvested at 4 weeks and 8 weeks post dosing and vector genome copy and mRNA levels were determined.

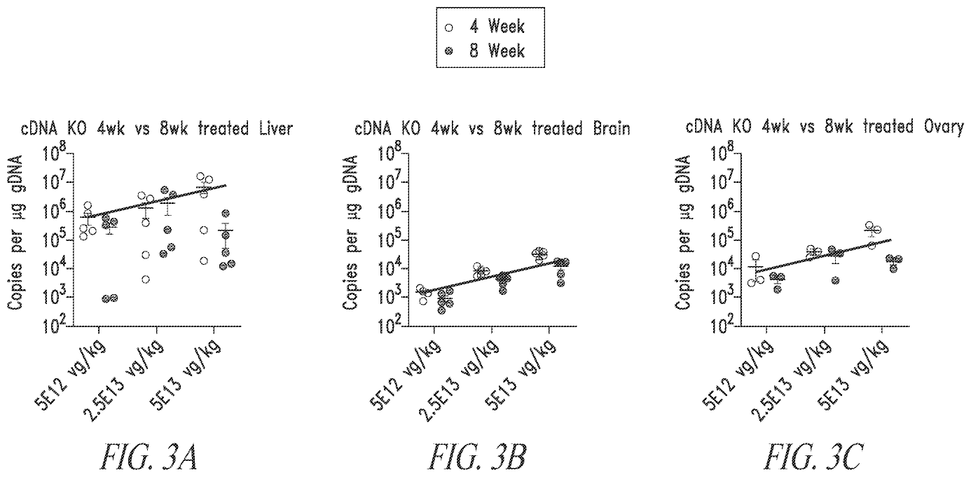

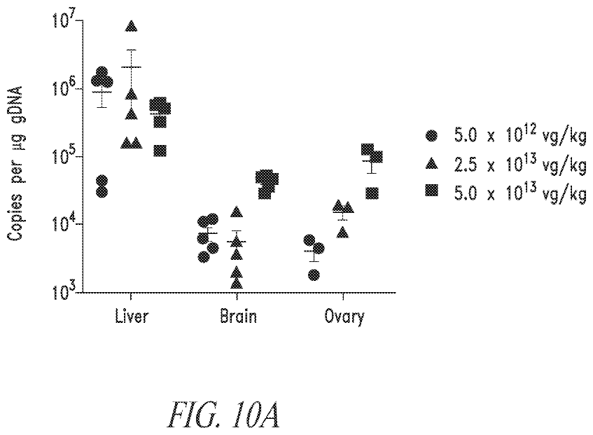

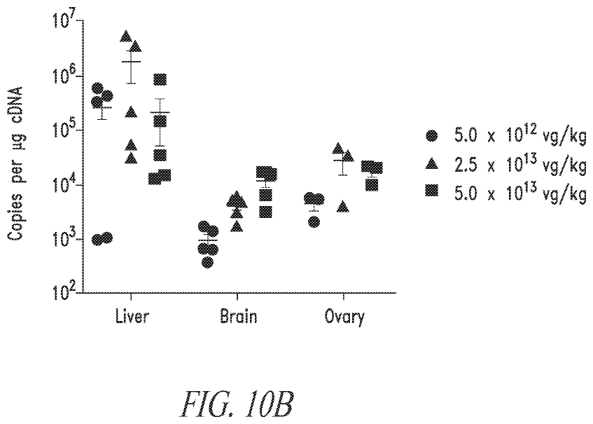

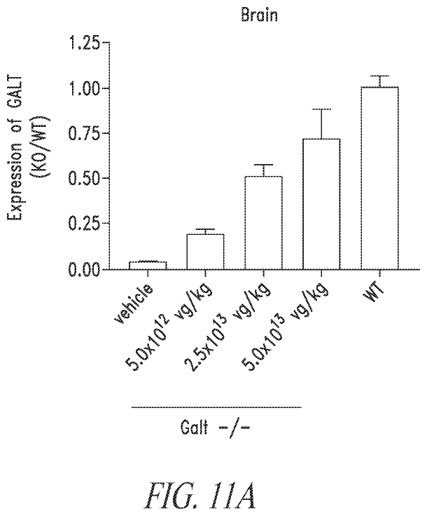

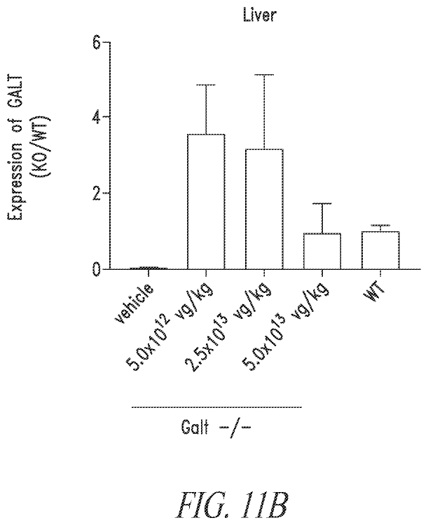

The results are shown in FIGS. 3A-3C and FIGS. 10A-10B. FIGS. 3A-3C show GALT mRNA copies per μg of cDNA of liver tissue, brain tissue, and ovary tissue at 4 and 8 weeks, while FIGS. 10A-10B show vector genomes (FIG. 10A) and transgene RNA (FIG. 10B) copies per μg of cDNA of liver, brain, and ovary tissue after dosing of mice at 5.0×10¹² vg/kg, 2.5×10¹³ vg/kg, and 5.0×10¹³ vg/kg. The results show that the GALT mRNA is expressed in each of the tissues tested. Moreover, the amount of GALT mRNA expressed in these tissues correlates with the dose administered, and is persistent at least until 8 weeks post dosing.

### Example 3: Expression of GALT Protein in GALT KO Mouse Model of Galactosemia

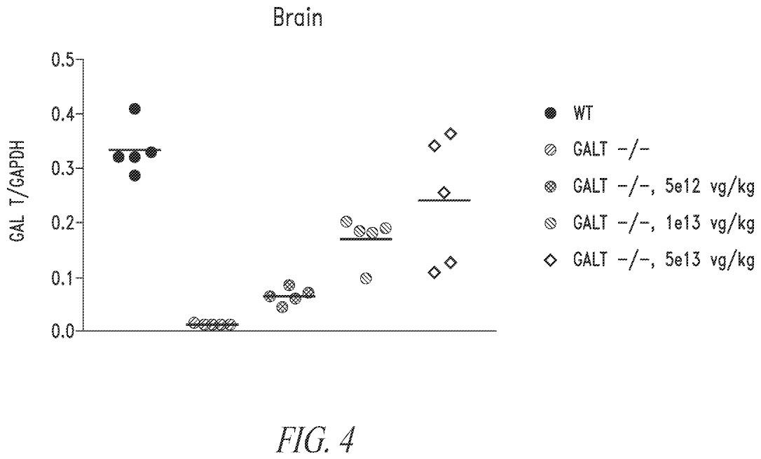

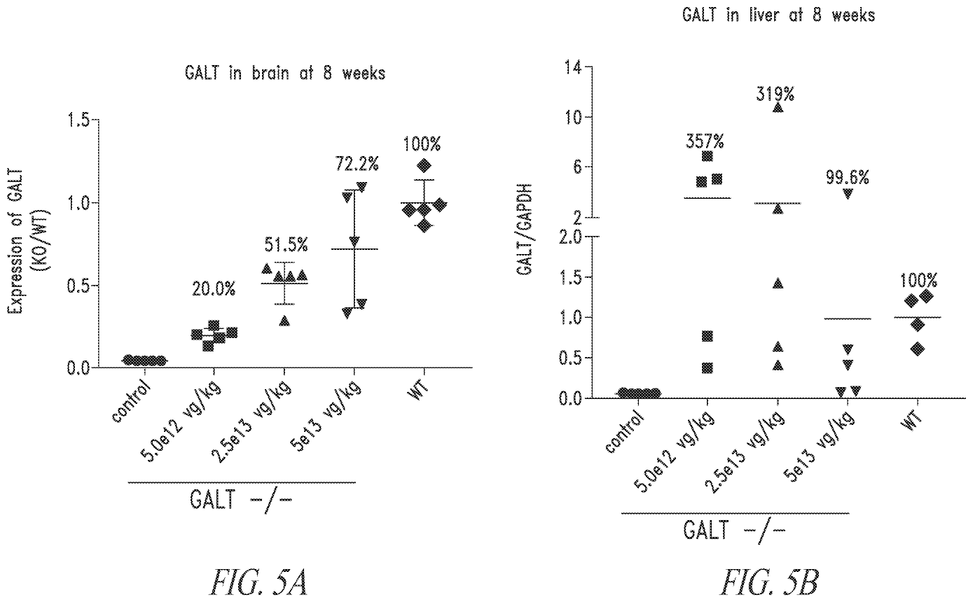

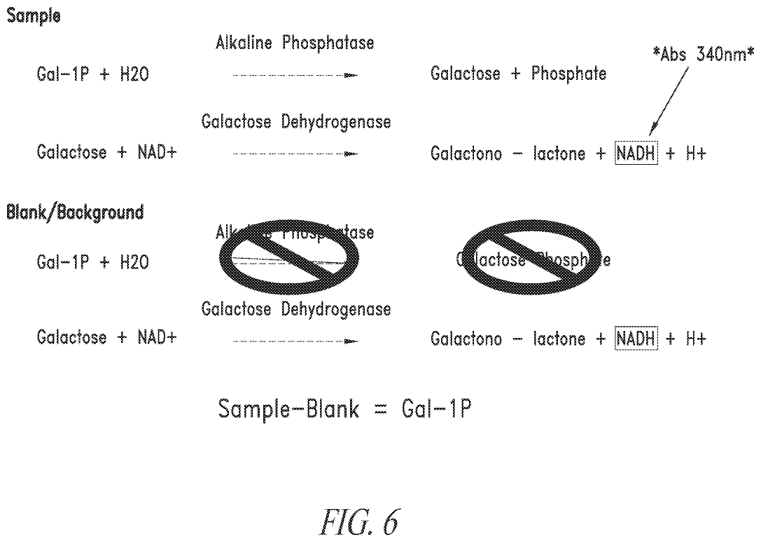

Ten week-old mice were injected intravenously with three different doses of the SCAAV-GALT (5.00×10¹² vg/kg: 2.50×10¹³ vg/kg: or 5.00×10¹³ vg/kg). Brain tissue was harvested at 8 weeks post dosing and brain homogenates were analyzed using a sodium dodecyl sulfate-polyacrylamide gel electrophoresis (SDS-PAGE) followed by transfer to a membrane and immunoblotting with an antibody that detects GALT protein. The amount of protein was quantitated using densitometry on the protein bands. The results are shown in FIG. 4. The results show that the GALT knockout mice treated with the scAAV-GALT therapy disclosed herein produced GALT protein in the brain in a dose-dependent manner. For instance, GALT knockout mice that were administered the highest tested dose of scAAV-GALT expressed as much as about 72% of wildtype GALT protein levels. These results indicate that the AAV-based gene therapy compositions and methods disclosed herein can be used to restore GALT protein levels in vivo, and hence treat galactosemia.

Further. GALT protein expression in the brain and liver of knockout mice was analyzed relative to WT mice at 8 weeks post dosing. The results are shown in FIGS. 5A-5B and FIGS. 11A-11B. The results show that the GALT knockout mice treated with the scAAV-GALT therapy disclosed herein produced as much or even more GALT protein in the brain and liver, as compared to the WT mice.

### Example 4: Reduction in the Level of Galactose-1-Phosphate (Gal-1-P) in GALT KO Mouse Model of Galactosemia

Ten week-old mice were injected intravenously with three different doses of the SCAAV-GALT (5.0×10¹² vg/kg: or 2.50×10¹³ vg/kg: or 5.0×10¹³ vg/kg), and liver, brain and red blood cells were harvested at 4 and 8 weeks post dosing. The level of Gal-1-P in the tissue samples was examined using an enzymatic assay described in FIG. 6. Briefly, the method involves the use of alkaline phosphatase and galactose dehydrogenase to convert free Gal-1-P to 1,4-dihydronicotinamide adenine dinucleotide (NADH) in a 1:1 fashion where it can then be quantified by measuring absorbance at 340 nanometers (nm). Many galactosemia diagnostics employ a similar strategy to measure the total galactose+Gal-1-P present in a patient's blood sample. One key difference in this method in particular, is that the amount of Gal-1-P can be quantified specifically. By removing the alkaline phosphatase from a duplicate sample, a background measurement of galactose can be carried out. This amount can then be subtracted from the total sample to quantify Gal-1-P levels specifically.

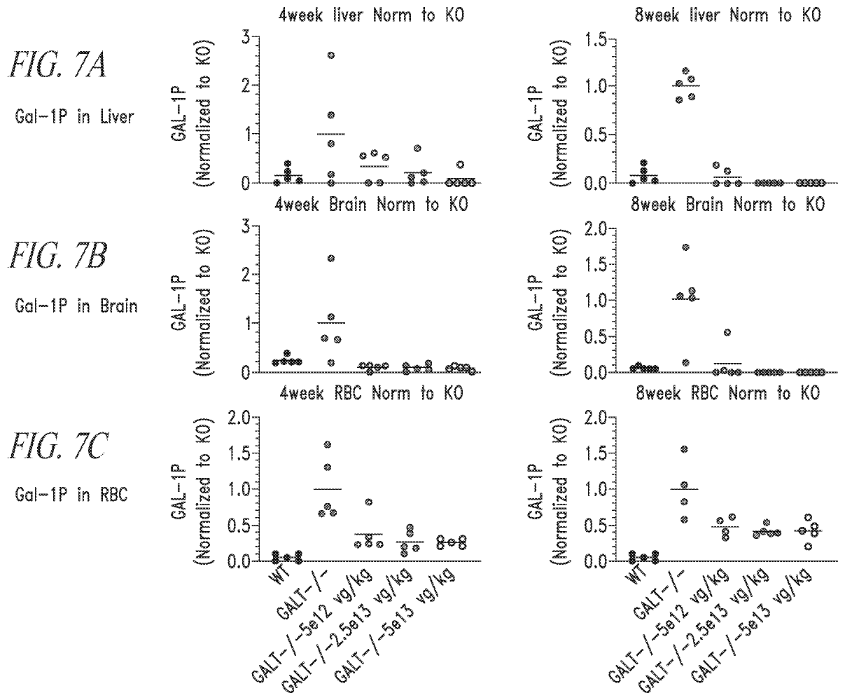

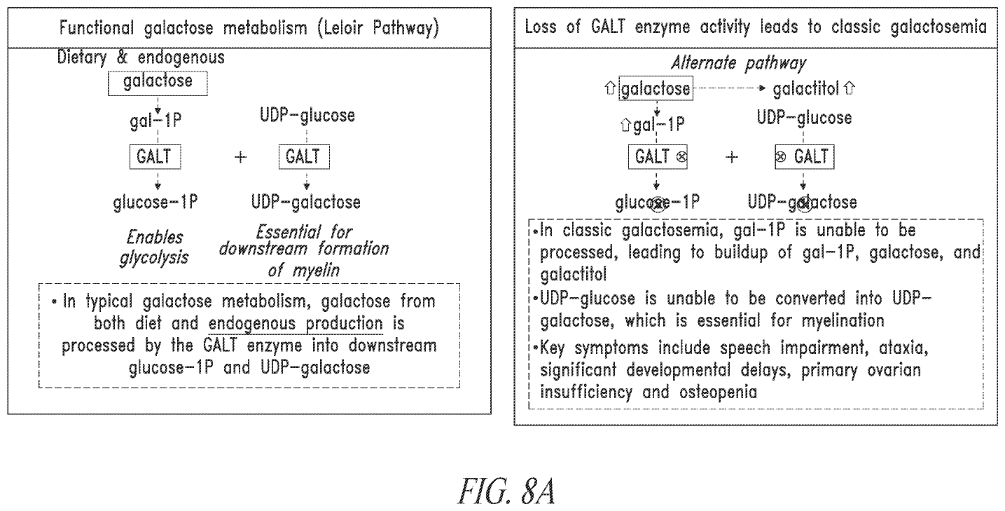

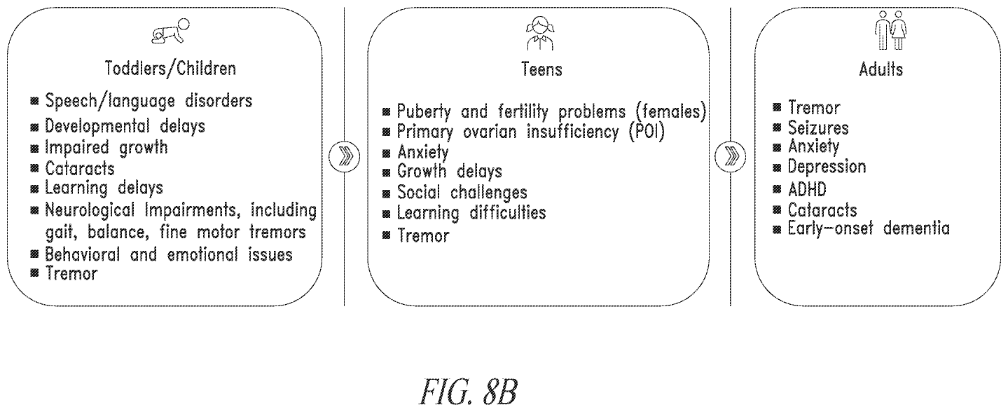

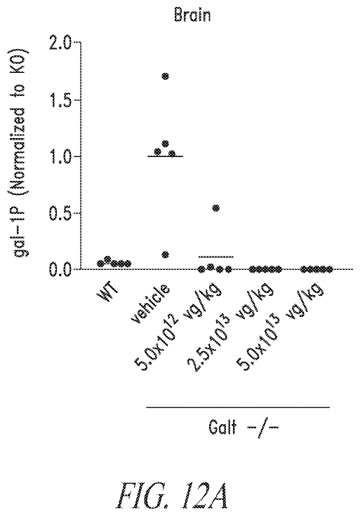

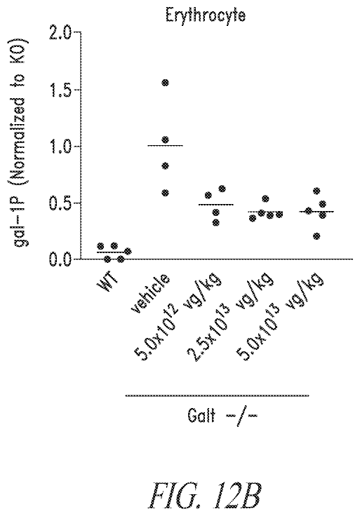

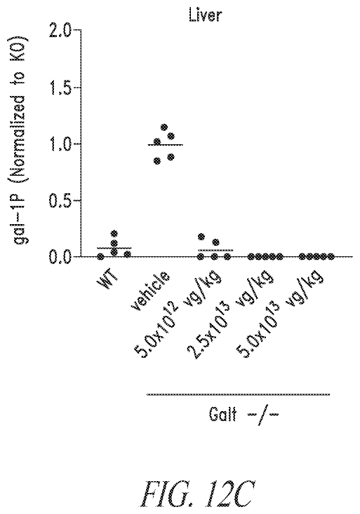

As shown in FIGS. 7A-7C and FIGS. 12A-12C, the level of Gal-1-P in the treated mice are significantly reduced relative to untreated control animals. These results demonstrate that the administration of the scAAV-GALT disclosed herein is able to successfully restore the function of GALT enzyme, reducing the excess, toxic levels of galactose-1-phosphate in these animals. The Gal-1-P levels will be further confirmed by mass spectrometry (MS).

### Example 5: Alleviation of Galactosemia Symptoms in GALT KO Mouse Model

To evaluate the effectiveness of the gene therapy in treating galactosemia, the scAAV-GALT particles are administered to a GALT-knockout mouse model of galactosemia, and the mice are examined to determine the ability of the scAAVs to attenuate galactosemia-like phenotypes of these mice. Particularly, the survival, motor coordination, brain histology, ovarian histology, assessments of the central nervous system (CNS) and fertility of GALT KO mice treated with scAAV-GALT are examined, relative to control untreated GALT KO mice. Neonatal pup survival following galactose challenge is evaluated in GALT KO mice treated with scAAV-GALT, relative to control untreated GALT KO mice. Toxicity studies in non-human primates are also conducted.

### Example 6: Rescue of Subfertility Phenotype and Restoration of GALT Protein Expression in Galt −/− Mice

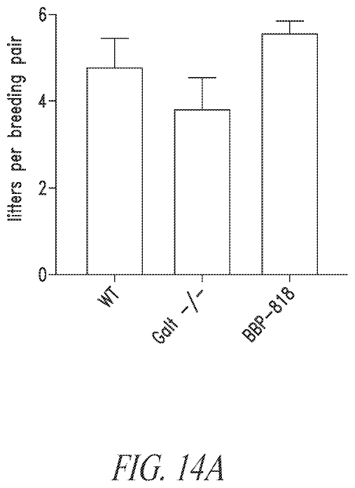

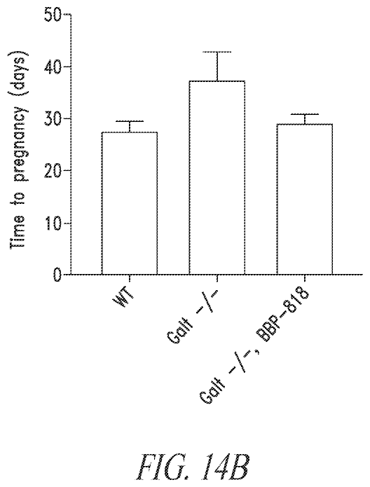

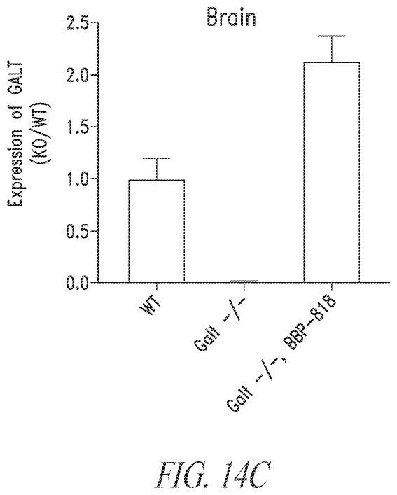

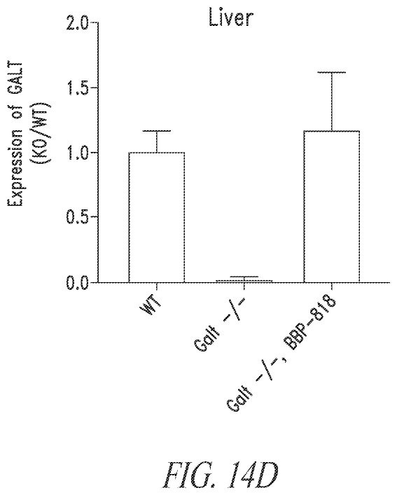

Mice were treated or untreated with scAAV-GALT particles as described in the preceding examples. Fertility was monitored until animals reached 200 days of age. Mice were monitored for litters per breeding pair (FIG. 14A) and time to pregnancy (FIG. 14B) throughout the study. At necropsy, tissues were collected and processed for biodistribution analysis including western blot analysis of protein expression. Relative GALT protein levels in the brain and liver are shown in FIGS. 14C and 14D. All data shown are mean±SEM of n=5 (WT) and n=6 (Galt −/− and Galt −/− with scAAV-GALT particles. As shown in FIGS. 14A-14D, the subfertility phenotype is rescued and GALT protein expression rescued in Galt −/− mice treated with scAAV-GALT particles.

### Example 7: Restoration of Galactose Metabolism in GALT KO Mouse Model

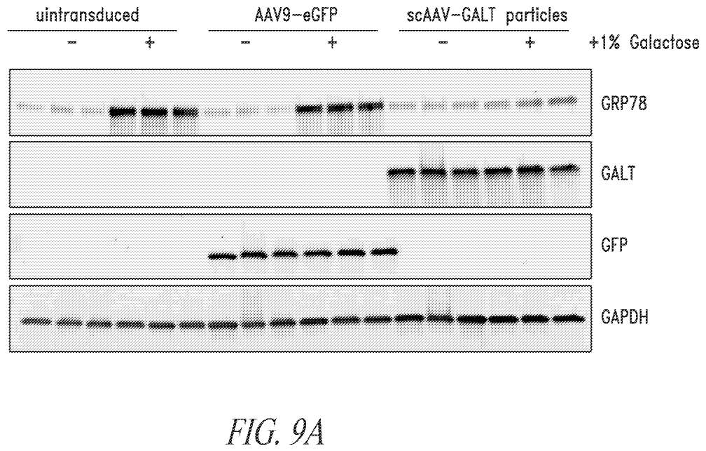

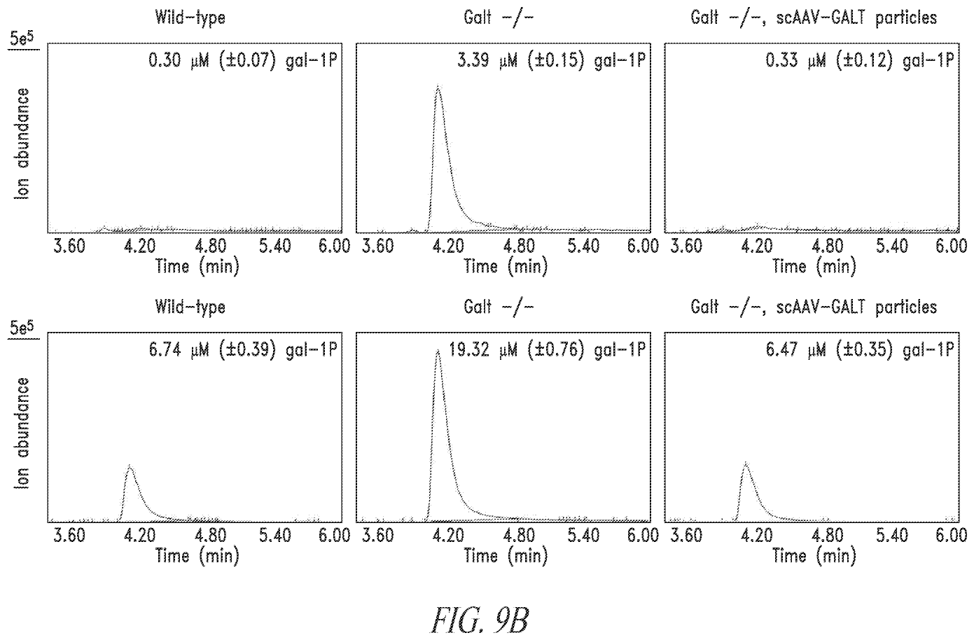

Galt −/− HEK293T cells were propagated in hexose-free media supplemented with 1% glucose or 1% galactose. Some cells were transduced with AAV9-GFP or scAAV-GALT particles. After 72 hours, lysates were harvested and processed for western blot analysis for GRP78 (marker of ER stress), GALT, GFP, and GAPDH (FIG. 9A). Galt −/− cells were propagated in hexose-free media supplemented with 1% galactose. After 24 hours, cells were processed for gal-1P measurement by LC-MS/MS analysis (FIG. 9B). These results show that scAAV-GALT particles restore galactose metabolism and reduces ER stress in Galt −/− HEK293T cells.

### Example 8: Transgene Expression in C57BI/6 Mouse Model

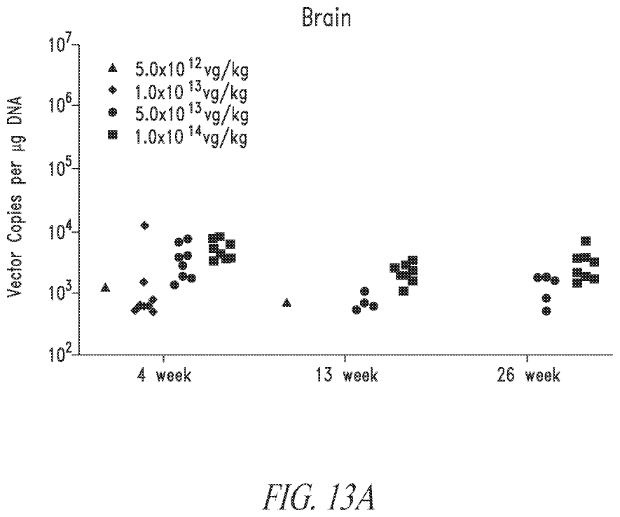

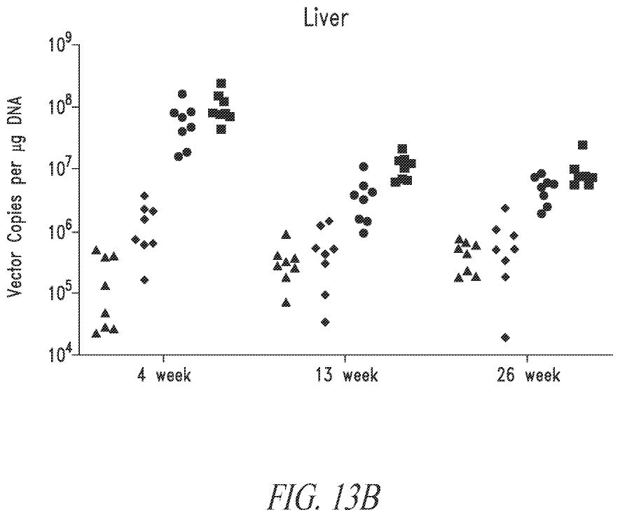

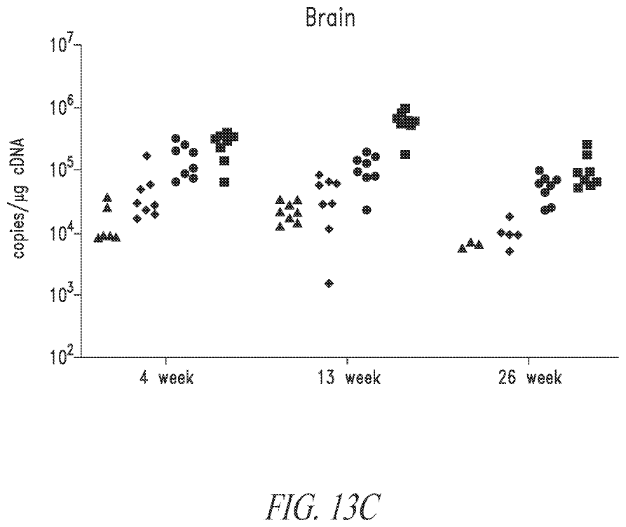

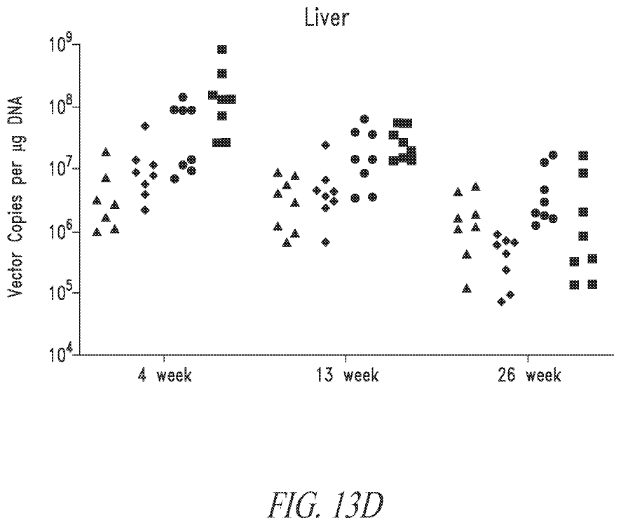

Transgene expression in a C57BI/6 mouse model was also explored to evaluate the durability of expression. The mice were prepared and transgene expression evaluated as described in preceding examples. Table 2 summarizes the details of the study design. Levels of vector genomes of the scAAV-GALT particles (FIGS. 13A-13B) and Galt transgene (FIGS. 13C-13D) in mouse brain and liver were measured with qPCR. Data shown in FIGS. 13A-13D are representative of n−8 per timepoint with an equal proportion of male and female animals. The LLOQ of each assay was 50 copies per reaction. There was no detection of vector or transgene in any untreated animals.

TABLE 2
Study design for evaluation of transgene
expression in C57BI/6 mouse model

| | | | Dose level | |
|---|---|---|---|---|
| Group | Genotype | Vector | (vg/kg) | Duration |
| 1 | C57BI/6 | Untreated | — | 4, 13, |
| 2 | | scAAV-GALT | 5.0 × 10¹² | 26, and |
| 3 | | particles | 1.0 × 10¹³ | 52 weeks |
| 4 | | | 5.0 × 10¹³ | |
| 5 | | | 1.0 × 10¹⁴ | |

Overall, Examples 1-8 show that scAAV-GALT particles restore galactose metabolism and tolerance in Galt −/− HEK293T cells. Doses of up to 1×10¹⁴ vg/kg have been well tolerated in mice with persistent transgene expression through 26 weeks post dosing. Gal-Ip levels in the brain and liver of Galt −/− mice were normalized with doses as low as 5×10¹² vg/kg. The GALT AAV therapy persists with complete recovery of GALT protein expression and normalization of gal-Ip levels in the brain and liver for at least 30 weeks in GALT −/− mice, and rescues the subfertility phenotype and improves motor function. These results and ongoing Investigative New Drug (IND)-enabling GLP-toxicology studies support continued development of this therapy for treatment of classic galactosemia.

### Example 9: Development of an In Vitro Potency Assay for Galactose-1-Phosphate Uridylyltransferase (GALT) Enzymatic Activity in AAV-Transduced Cells

The GALT enzyme catalyzes the reversible reaction between co-substrates gal-Ip and glucose-uridine-diphosphate (glu-UDP), wherein the uridine monophosphate group is transferred from glu-UDP to gal-IP to form co-products galactose-uridine-diphosphate (gal-UDP) and glucose-1-phosphate (glu-IP). To quantify the GALT reaction rates in cells transduced with sc-AAV GALT particles, an LC-MS/MS method was developed for quantification of the reaction product Gal-UDP.

Gal-1P and Glu-IP are monophosphorylated sugars differing only in the configuration of a single hydroxyl (encircled in green in the figure below), and thus, have identical molecular weights. Similarly. Glu-UDP and Gal-UDP, each containing an additional uridine-monophosphate group relative to their monophosphorylated sugar precursors, differ only in the same hydroxyl configuration.

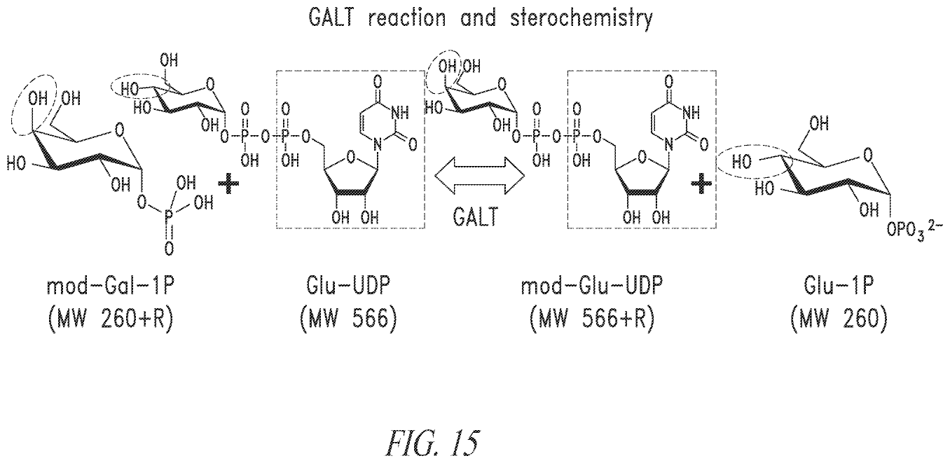

Thus, in order to distinguish between the reactants and products in the GALT system using LC-MS/MS, modified Gal-1P (mod-Gal-1P) was used as a substrate, driving formation of modified Gal-UDP (mod-Gal-UDP), which is distinguishable by mass from Glu-UDP. Using this approach, all substrates and products could be independently quantified. FIG. 15 shows the GALT reaction and stereochemistry of these components.

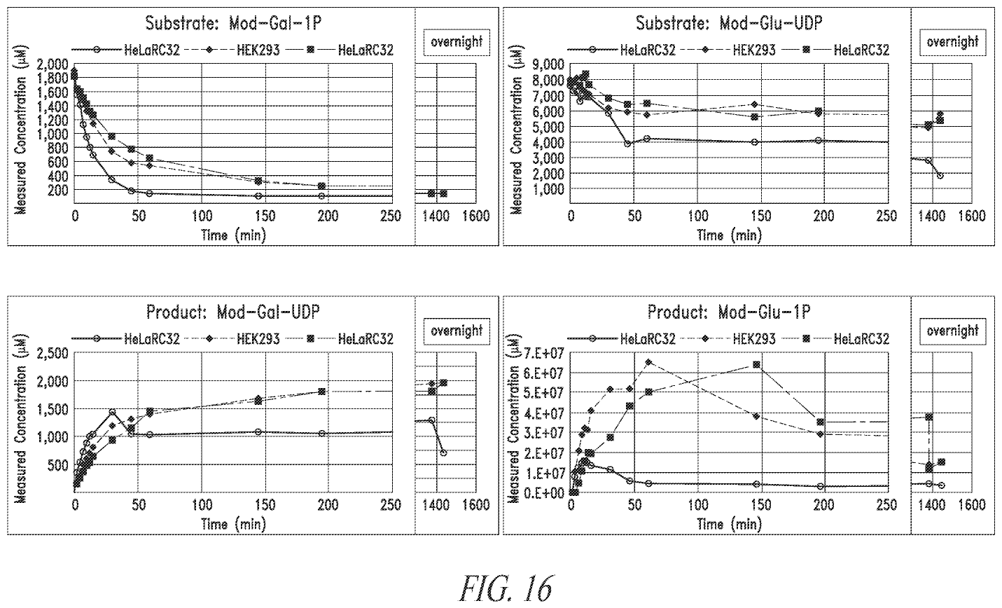

Quantification of substrates and products: In lysates of cells (HeLaRC32, HEK293, and HEK293ΔGALT) transduced with scAAV-GALT particles and spiked with 2 millimolar (mM) mod-Gal-IP and 8 mM Glu-UDP, approximately 2 mM of each substrate was consume, resulting in near complete consumption of mod-Gal-IP and reduction of Glu-UDP from 8 to approximately 6 mM. Concomitantly. Glu-IP is transiently produced and consumed while the relatively inert product mod-Gal-UDP is generated at approximately 2 mM (FIG. 16). From these observations, mod-Gal-UDP was selected as the marker for GALT reaction rates in in vitro activity assays.

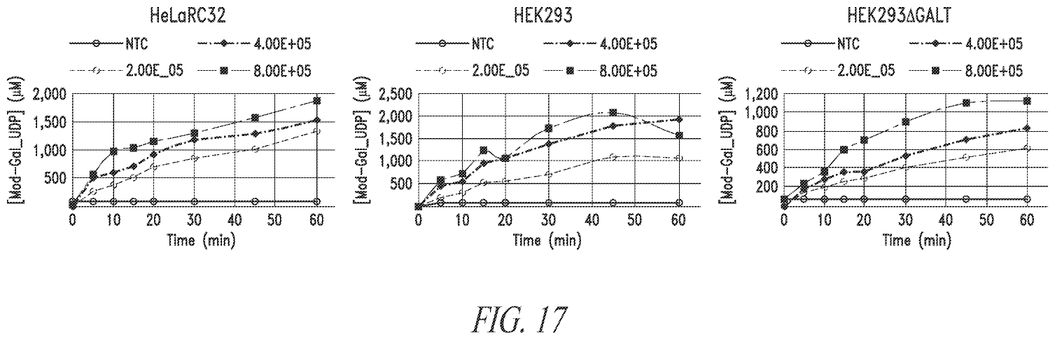

Dose-dependent GALT activity and assay specificity: In lysates of cells (HeLaRC32, HEK293, and HEK293ΔGALT) transduced with scAAV-GALT particles at MOIs of 200,000, 400,000, and 800,000 vg/cell, the rate of mod-Gal-UDP formation was not observed in non-infected cells hwereas GALT activity increases as a function of increasing MOI in cells transduced with the scAAV-GALT particles (FIG. 17).

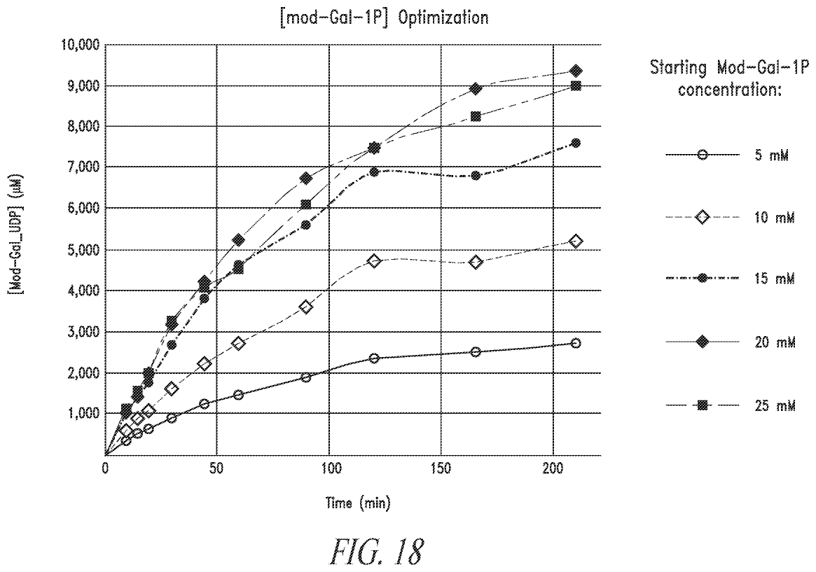

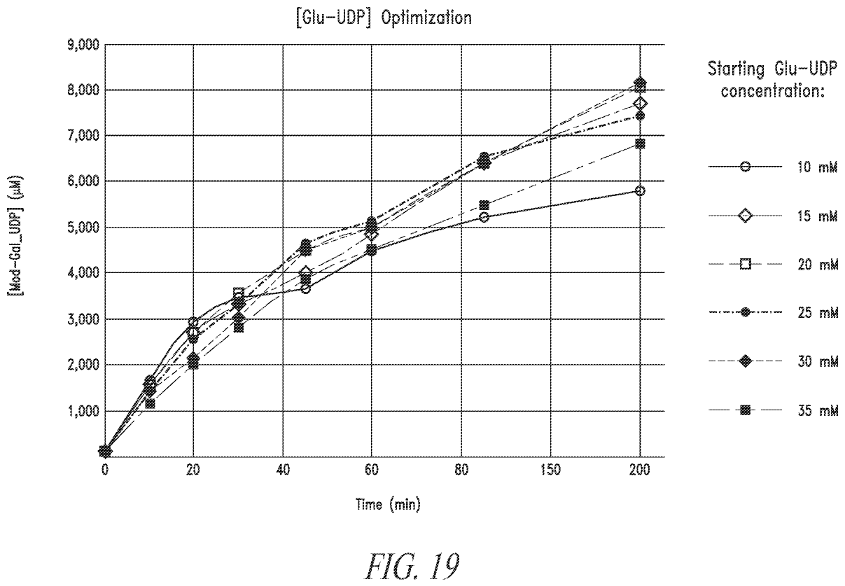

Optimization of substrate concentrations: Varying the spike concentration of mod-Gal-1P from 5 mM to 25 mM (at constant 30 mM Glu-UDP) revealed that 20 mM mod-Galt-IP was sufficient for saturating the enzymatic activity of the GALT enzyme expressed in HeLaRC32 cells transduced with the scAAV-GALT particles. Thus, 20 mM mod-Gal-IP was selected as the optimum substrate concentration (FIG. 18). GALT activity was monitored at a constant mod-Gal-1P of 20 mM and variable Glu-UDP from 10 to 35 mM. During the 120-minute reaction period, activity as saturated from 15 to 30 mM Glu-UDP, with a potential inhibitory effect at 35 mM. Thus, to be in excess of 20 mM mod-Gal-1P, 30 mM Glu-UDP was selected (FIG. 19).

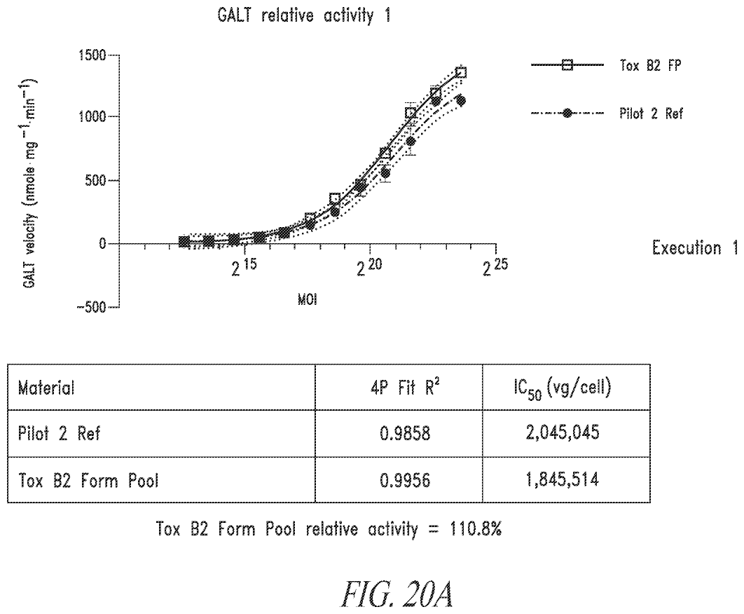

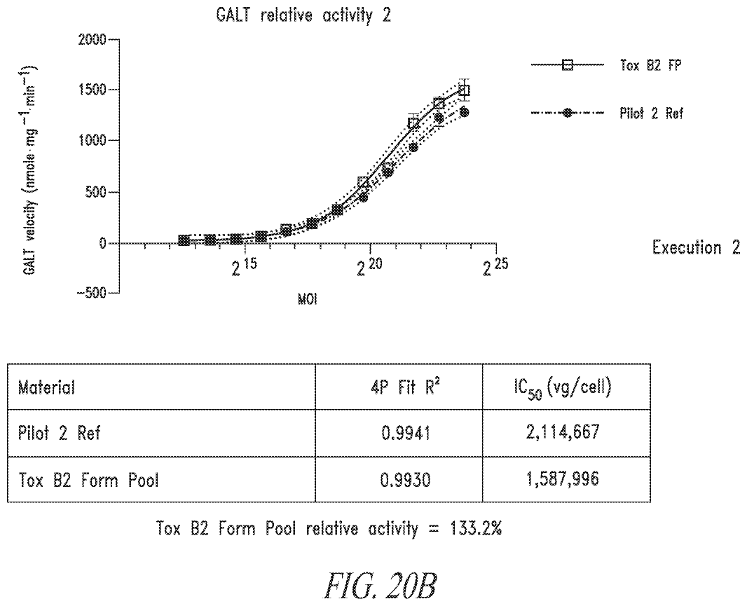

Relative activity and assay repeatability: To test repeatability of the GALT in vitro activity assay, two absolute activities of two scAAV-GALT particle batches were measured across MOIs from 6250 to 12,800,000 vg/cell. Relative activity was assessed as the ratio of IC50 concentrations between the two materials, revealing a high level of assay repeatability across multiple executions (FIGS. 20A-20B).

In summary, an LC-MS/MS method was developed for the quantification of each substrate and product in the GALT enzymatic reaction. Equimolar conversion of substrates to products was observed in multiple cell lines. In vitro activity assay specificity and dose dependence for scAAV-GALT particles was demonstrated. Substrate concentrations were optimized to enable single time point evaluation of enzymatic velocity across a broad range of MOIs. The activity assay exhibited a high level of repeatability across multiple executions, demonstrating the utility of the assay in supporting the development of scAAV-GALT particles and assessing lot-to-lot consistency.

Table 3 below lists the nucleic acid and protein sequences provided in the present disclosure.

TABLE 3
Nucleic acid and Protein sequences of the present disclosure

| SEQ ID NO: | Description | Sequence |
|---|---|---|
| 1 | Codon optimized | ATGAGCCGGAGCGGCACCGACCCCCAGCAGCGG |
| | GALT coding | CAGCAGGCCAGCGAGGCCGACGCTGCCGCCGCC |
| | sequence | ACCTTCCGGGCCAACGACCACCAGCACATCCGG |
| | | TACAACCCCCTGCAGGACGAGTGGGTGCTGGTG |
| | | AGCGCCCACCGGATGAAGCGGCCCTGGCAGGGC |
| | | CAGGTGGAGCCCCAGCTGCTGAAGACCGTGCCC |
| | | CGGCACGACCCCCTGAACCCTCTGTGCCCAGGA |
| | | GCCATCCGCGCCAACGGCGAGGTGAATCCTCAG |
| | | TACGACAGCACCTTCCTGTTCGACAACGACTTCC |
| | | CCGCCCTGCAGCCCGACGCCCCCAGCCCCGGCC |
| | | CCAGCGACCATCCATTATTCCAGGCCAAGAGCG |
| | | CCCGGGGCGTGTGCAAGGTGATGTGCTTCCACC |
| | | CCTGGAGCGACGTGACCCTGCCCCTGATGAGCG |
| | | TGCCCGAGATCAGAGCCGTGGTGGACGCCTGGG |
| | | CCAGCGTGACCGAGGAGCTGGGCGCCCAGTACC |
| | | CCTGGGTGCAGATCTTCGAGAACAAGGGCGCCA |
| | | TGATGGGCTGCAGCAACCCCCACCCCCACTGCC |
| | | AGGTGTGGGCCTCAAGCTTCCTGCCCGACATCGC |
| | | CCAGCGGGAGGAGCGGAGCCAACAGGCCTACAA |
| | | GTCACAGCACGGCGAGCCCCTGCTGATGGAGTA |
| | | CAGCCGGCAGGAGCTGCTGCGGAAGGAGCGGCT |
| | | GGTGCTGACCAGCGAGCACTGGCTCGTGCTCGT |
| | | GCCCTTCTGGGCCACCTGGCCCTACCAGACCCTG |
| | | CTGCTGCCTAGACGGCACGTGCGGCGGCTGCCC |
| | | GAGCTGACCCCCGCCGAGCGGGACGACCTGGCC |
| | | AGCATCATGAAGAAGCTCCTGACCAAGTACGAT |
| | | AACTTATTCGAAACAAGCTTCCCCTACAGCATGG |
| | | GCTGGCACGGCGCCCCCACCGGCTCAGAGGCCG |
| | | GCGCTAACTGGAACCACTGGCAGCTGCACGCCC |
| | | ACTACTACCCGCCTCTGCTGAGAAGCGCCACCGT |
| | | GCGGAAGTTCATGGTGGGCTACGAGATGCTGGC |
| | | CCAGGCCCAGAGAGACTTAACCCCTGAGCAGGC |
| | | CGCCGAGAGGCTCCGGGCCTTGCCAGAGGTGCA |
| | | CTACCACCTGGGACAGAAGGACCGGGAGACGGC |
| | | CACCATCGCCTGA |
| 2 | CB6 promoter | CCACGTTCTGCTTCACTCTCCCCATCTCCCCCCC |
| | | CTCCCCACCCCCAATTTTGTATTTATTTATTTTTT |
| | | AATTATTTTGTGCAGCGATGGGGGCGGGGGGGG |
| | | GGGGCGCGCGCCAGGCGGGGGGGGGCGGGGCGA |
| | | GGGGGGGGCGGGGCGAGGCGGAGAGGTGCGGC |
| | | GGCAGCCAATCAGAGCGGCGCGCTCCGAAAGTT |
| | | TCCTTTTATGGCGAGGCGGCGGCGGCGGCGGCC |
| | | CTATAAAAAGCGAAGCGCGCGGCGGG |
| 3 | rabbit beta-globin | GATCTTTTTCCCTCTGCCAAAAATTATGGGGACA |
| | polyadenylation | TCATGAAGCCCCTTGAGCATCTGACTTCTGGCTA |
| | sequence | ATAAAGGAAATTTATTTTCATTGCAATAGTGTGT |
| | | TGGAATTTTTTGTGTCTCTCACTCG |
| 4 | CMV-IE enhancer | CGCGTCGACATTGATTATTGACTAGTTATTAATA |
| | | GTAATCAATTACGGGGTCATTAGTTCATAGCCCA |
| | | TATATGGAGTTCCGCGTTACATAACTTACGGTAA |
| | | ATGGCCCGCCTGGCTGACCGCCCAACGACCCCC |
| | | GCCCATTGACGTCAATAATGACGTATGTTCCCAT |
| | | AGTAACGCCAATAGGGACTTTCCATTGACGTCA |
| | | ATGGGTGGAGTATTTACGGTAAACTGCCCACTTG |
| | | GCAGTACATCAAGTGTATCATATGCCAAGTACG |
| | | CCCCCTATTGACGTCAATGACGGTAAATGGCCC |
| | | GCCTGGCATTATGCCCAGTACATGACCTTATGGG |
| | | ACTTTCCTACTTGGCAGTACATCTACGTATTAGT |
| | | CATCGCTATTAC |
| 5 | Kozak sequence | GCCACC |
| 6 | 5′ ITR | CTGCGCGCTCGCTCGCTCACTGAGGCCGCCCGG |
| | | GCAAAGCCCGGGCGTCGGGCGACCTTTGGTCGC |
| | | CCGGCCTCAGTGAGCGAGCGAGCGCGCAGAGAG |
| | | GGAGTG |
| 7 | 3′ ITR | AGGAACCCCTAGTGATGGAGTTGGCCACTCCCT |
| | | CTCTGCGCGCTCGCTCGCTCACTGAGGCCGGGCG |
| | | ACCAAAGGTCGCCCGACGCCCGGGCTTTGCCCG |
| | | GGCGGCCTCAGTGAGCGAGCGAGCGCGCAG |
| 8 | Full sequence of | CTGCGCGCTCGCTCGCTCACTGAGGCCGCCCGG |
| | the AAV | GCAAAGCCCGGGCGTCGGGCGACCTTTGGTCGC |
| | expression | CCGGCCTCAGTGAGCGAGCGAGCGCGCAGAGAG |
| | cassette | GGAGTGTAGCCATGCTCTAGGAAGATCAATTCA |
| | | ATTCACGCGTCGACATTGATTATTGACTAGTTAT |
| | | TAATAGTAATCAATTACGGGGTCATTAGTTCATA |
| | | GCCCATATATGGAGTTCCGCGTTACATAACTTAC |
| | | GGTAAATGGCCCGCCTGGCTGACCGCCCAACGA |
| | | CCCCCGCCCATTGACGTCAATAATGACGTATGTT |
| | | CCCATAGTAACGCCAATAGGGACTTTCCATTGAC |
| | | GTCAATGGGTGGAGTATTTACGGTAAACTGCCC |
| | | ACTTGGCAGTACATCAAGTGTATCATATGCCAA |
| | | GTACGCCCCCTATTGACGTCAATGACGGTAAAT |
| | | GGCCCGCCTGGCATTATGCCCAGTACATGACCTT |
| | | ATGGGACTTTCCTACTTGGCAGTACATCTACGTA |
| | | TTAGTCATCGCTATTACCATGTCGAGGCCACGTT |
| | | CTGCTTCACTCTCCCCATCTCCCCCCCCTCCCCA |
| | | CCCCCAATTTTGTATTTATTTATTTTTTAATTATT |
| | | TTGTGCAGCGATGGGGGCGGGGGGGGGGGGCGC |
| | | GCGCCAGGCGGGGGGGGGCGGGGCGAGGGGCGG |
| | | GGCGGGGCGAGGCGGAGAGGTGCGGCGGCAGCC |
| | | AATCAGAGCGGCGCGCTCCGAAAGTTTCCTTTTA |
| | | TGGCGAGGCGGCGGCGGCGGCGGCCCTATAAAA |
| | | AGCGAAGCGCGCGGCGGGCGGGAGCAAGCTCTA |
| | | GCCTCGAGAATTCGCCACCATGAGCCGGAGCGG |
| | | CACCGACCCCCAGCAGCGGCAGCAGGCCAGCGA |
| | | GGCCGACGCTGCCGCCGCCACCTTCCGGGCCAA |
| | | CGACCACCAGCACATCCGGTACAACCCCCTGCA |
| | | GGACGAGTGGGTGCTGGTGAGCGCCCACCGGAT |
| | | GAAGCGGCCCTGGCAGGGCCAGGTGGAGCCCCA |
| | | GCTGCTGAAGACCGTGCCCCGGCACGACCCCCT |
| | | GAACCCTCTGTGCCCAGGAGCCATCCGCGCCAA |
| | | CGGCGAGGTGAATCCTCAGTACGACAGCACCTT |
| | | CCTGTTCGACAACGACTTCCCCGCCCTGCAGCCC |
| | | GACGCCCCCAGCCCCGGCCCCAGCGACCATCCA |
| | | TTATTCCAGGCCAAGAGCGCCCGGGGCGTGTGC |
| | | AAGGTGATGTGCTTCCACCCCTGGAGCGACGTG |
| | | ACCCTGCCCCTGATGAGCGTGCCCGAGATCAGA |
| | | GCCGTGGTGGACGCCTGGGCCAGCGTGACCGAG |
| | | GAGCTGGGCGCCCAGTACCCCTGGGTGCAGATC |
| | | TTCGAGAACAAGGGCGCCATGATGGGCTGCAGC |
| | | AACCCCCACCCCCACTGCCAGGTGTGGGCCTCA |
| | | AGCTTCCTGCCCGACATCGCCCAGCGGGAGGAG |
| | | CGGAGCCAACAGGCCTACAAGTCACAGCACGGC |
| | | GAGCCCCTGCTGATGGAGTACAGCCGGCAGGAG |
| | | CTGCTGCGGAAGGAGCGGCTGGTGCTGACCAGC |
| | | GAGCACTGGCTCGTGCTCGTGCCCTTCTGGGCCA |
| | | CCTGGCCCTACCAGACCCTGCTGCTGCCTAGACG |
| | | GCACGTGCGGCGGCTGCCCGAGCTGACCCCCGC |
| | | CGAGCGGGACGACCTGGCCAGCATCATGAAGAA |
| | | GCTCCTGACCAAGTACGATAACTTATTCGAAAC |
| | | AAGCTTCCCCTACAGCATGGGCTGGCACGGCGC |
| | | CCCCACCGGCTCAGAGGCCGGCGCTAACTGGAA |
| | | CCACTGGCAGCTGCACGCCCACTACTACCCGCCT |
| | | CTGCTGAGAAGCGCCACCGTGCGGAAGTTCATG |
| | | GTGGGCTACGAGATGCTGGCCCAGGCCCAGAGA |
| | | GACTTAACCCCTGAGCAGGCCGCCGAGAGGCTC |
| | | CGGGCCTTGCCAGAGGTGCACTACCACCTGGGA |
| | | CAGAAGGACCGGGAGACGGCCACCATCGCCTGA |
| | | GGTACCTCTAGAGTCGACCCGGGCGGCCTCGAG |
| | | GACGGGGTGAACTACGCCTGAGGATCCGATCTT |
| | | TTTCCCTCTGCCAAAAATTATGGGGACATCATGA |
| | | AGCCCCTTGAGCATCTGACTTCTGGCTAATAAAG |
| | | GAAATTTATTTTCATTGCAATAGTGTGTTGGAAT |
| | | TTTTTGTGTCTCTCACTCGGCCTAGGTAGATAAG |
| | | TAGCATGGCGGGTTAATCATTAACTACAAGGAA |
| | | CCCCTAGTGATGGAGTTGGCCACTCCCTCTCTGC |
| | | GCGCTCGCTCGCTCACTGAGGCCGGGCGACCAA |
| | | AGGTCGCCCGACGCCCGGGCTTTGCCCGGGCGG |
| | | CCTCAGTGAGCGAGCGAGCGCGCAG |
| 9 | RNA Kozak | ACCAUGG |
| | sequence-1 | |
| 10 | RNA Kozak | GCCGCCACCAUGG |
| | sequence-2 | |
| 11 | RNA Kozak | CCACCAUG |
| | sequence-3 | |
| 12 | RNA Kozak | CCACCAUGG |
| | sequence-4 | |
| 13 | 3602 plasmid | CTGCGCGCTCGCTCGCTCACTGAGGCCGCCCGG |
| | comprising the AAV | GCAAAGCCCGGGCGTCGGGCGACCTTTGGTCGC |
| | expression cassette | CCGGCCTCAGTGAGCGAGCGAGCGCGCAGAGAG |
| | of SEQ ID NO: 8 | GGAGTGTAGCCATGCTCTAGGAAGATCAATTCA |
| | | ATTCACGCGTCGACATTGATTATTGACTAGTTAT |
| | | TAATAGTAATCAATTACGGGGTCATTAGTTCATA |
| | | GCCCATATATGGAGTTCCGCGTTACATAACTTAC |
| | | GGTAAATGGCCCGCCTGGCTGACCGCCCAACGA |
| | | CCCCCGCCCATTGACGTCAATAATGACGTATGTT |
| | | CCCATAGTAACGCCAATAGGGACTTTCCATTGAC |
| | | GTCAATGGGTGGAGTATTTACGGTAAACTGCCC |
| | | ACTTGGCAGTACATCAAGTGTATCATATGCCAA |
| | | GTACGCCCCCTATTGACGTCAATGACGGTAAAT |
| | | GGCCCGCCTGGCATTATGCCCAGTACATGACCTT |
| | | ATGGGACTTTCCTACTTGGCAGTACATCTACGTA |
| | | TTAGTCATCGCTATTACCATGTCGAGGCCACGTT |
| | | CTGCTTCACTCTCCCCATCTCCCCCCCCTCCCCA |
| | | CCCCCAATTTTGTATTTATTTATTTTTTAATTATT |
| | | TTGTGCAGCGATGGGGGCGGGGGGGGGGGGCGC |
| | | GCGCCAGGCGGGGCGGGGCGGGGCGAGGGGCGG |
| | | GGCGGGGCGAGGCGGAGAGGTGCGGCGGCAGCC |
| | | AATCAGAGCGGCGCGCTCCGAAAGTTTCCTTTTA |
| | | TGGCGAGGCGGCGGCGGCGGCGGCCCTATAAAA |
| | | AGCGAAGCGCGCGGCGGGCGGGAGCAAGCTCTA |
| | | GCCTCGAGAATTCGCCACCATGAGCCGGAGCGG |
| | | CACCGACCCCCAGCAGCGGCAGCAGGCCAGCGA |
| | | GGCCGACGCTGCCGCCGCCACCTTCCGGGCCAA |
| | | CGACCACCAGCACATCCGGTACAACCCCCTGCA |
| | | GGACGAGTGGGTGCTGGTGAGCGCCCACCGGAT |
| | | GAAGCGGCCCTGGCAGGGCCAGGTGGAGCCCCA |
| | | GCTGCTGAAGACCGTGCCCCGGCACGACCCCCT |
| | | GAACCCTCTGTGCCCAGGAGCCATCCGCGCCAA |
| | | CGGCGAGGTGAATCCTCAGTACGACAGCACCTT |
| | | CCTGTTCGACAACGACTTCCCCGCCCTGCAGCCC |
| | | GACGCCCCCAGCCCCGGCCCCAGCGACCATCCA |
| | | TTATTCCAGGCCAAGAGCGCCCGGGGCGTGTGC |
| | | AAGGTGATGTGCTTCCACCCCTGGAGCGACGTG |
| | | ACCCTGCCCCTGATGAGCGTGCCCGAGATCAGA |
| | | GCCGTGGTGGACGCCTGGGCCAGCGTGACCGAG |
| | | GAGCTGGGCGCCCAGTACCCCTGGGTGCAGATC |
| | | TTCGAGAACAAGGGCGCCATGATGGGCTGCAGC |
| | | AACCCCCACCCCCACTGCCAGGTGTGGGCCTCA |
| | | AGCTTCCTGCCCGACATCGCCCAGCGGGAGGAG |
| | | CGGAGCCAACAGGCCTACAAGTCACAGCACGGC |
| | | GAGCCCCTGCTGATGGAGTACAGCCGGCAGGAG |
| | | CTGCTGCGGAAGGAGCGGCTGGTGCTGACCAGC |
| | | GAGCACTGGCTCGTGCTCGTGCCCTTCTGGGCCA |
| | | CCTGGCCCTACCAGACCCTGCTGCTGCCTAGACG |
| | | GCACGTGCGGCGGCTGCCCGAGCTGACCCCCGC |
| | | CGAGCGGGACGACCTGGCCAGCATCATGAAGAA |
| | | GCTCCTGACCAAGTACGATAACTTATTCGAAAC |
| | | AAGCTTCCCCTACAGCATGGGCTGGCACGGCGC |
| | | CCCCACCGGCTCAGAGGCCGGCGCTAACTGGAA |
| | | CCACTGGCAGCTGCACGCCCACTACTACCCGCCT |
| | | CTGCTGAGAAGCGCCACCGTGCGGAAGTTCATG |
| | | GTGGGCTACGAGATGCTGGCCCAGGCCCAGAGA |
| | | GACTTAACCCCTGAGCAGGCCGCCGAGAGGCTC |
| | | CGGGCCTTGCCAGAGGTGCACTACCACCTGGGA |
| | | CAGAAGGACCGGGAGACGGCCACCATCGCCTGA |
| | | GGTACCTCTAGAGTCGACCCGGGCGGCCTCGAG |
| | | GACGGGGTGAACTACGCCTGAGGATCCGATCTT |
| | | TTTCCCTCTGCCAAAAATTATGGGGACATCATGA |
| | | AGCCCCTTGAGCATCTGACTTCTGGCTAATAAAG |
| | | GAAATTTATTTTCATTGCAATAGTGTGTTGGAAT |
| | | TTTTTGTGTCTCTCACTCGGCCTAGGTAGATAAG |
| | | TAGCATGGCGGGTTAATCATTAACTACAAGGAA |
| | | CCCCTAGTGATGGAGTTGGCCACTCCCTCTCTGC |
| | | GCGCTCGCTCGCTCACTGAGGCCGGGCGACCAA |
| | | AGGTCGCCCGACGCCCGGGCTTTGCCCGGGCGG |
| | | CCTCAGTGAGCGAGCGAGCGCGCAGCCTTAATTA |
| | | ACCTAATTCACTGGCCGTCGTTTTACAACGTCGT |
| | | GACTGGGAAAACCCTGGCGTTACCCAACTTAAT |
| | | CGCCTTGCAGCACATCCCCCTTTCGCCAGCTGGC |
| | | GTAATAGCGAAGAGGCCCGCACCGATCGCCCTT |
| | | CCCAACAGTTGCGCAGCCTGAATGGCGAATGGG |
| | | ACGCGCCCTGTAGCGGCGCATTAAGCGCGGCGG |
| | | GTGTGGTGGTTACGCGCAGCGTGACCGCTACACT |
| | | TGCCAGCGCCCTAGCGCCCGCTCCTTTCGCTTTC |
| | | TTCCCTTCCTTTCTCGCCACGTTCGCCGGCTTTC |
| | | CCCGTCAAGCTCTAAATCGGGGGCTCCCTTTAGG |
| | | GTTCCGATTTAGTGCTTTACGGCACCTCGACCCC |
| | | AAAAAACTTGATTAGGGTGATGGTTCACGTAGT |
| | | GGGCCATCGCCCTGATAGACGGTTTTTCGCCCTT |
| | | TGACGTTGGAGTCCACGTTCTTTAATAGTGGACT |
| | | CTTGTTCCAAACTGGAACAACACTCAACCCTATC |
| | | TCGGTCTATTCTTTTGATTTATAAGGGATTTTGCC |
| | | GATTTCGGCCTATTGGTTAAAAAATGAGCTGATT |
| | | TAACAAAAATTTAACGCGAATTTTAACAAAATA |
| | | TTAACGCTTACAATTTAGGTGGCACTTTTCGGGG |
| | | AAATGTGCGCGGAACCCCTATTTGTTTATTTTTC |
| | | TAAATACATTCAAATATGTATCCGCTCATGAGAC |
| | | AATAACCCTGATAAATGCTTCAATAATATTGAA |
| | | AAAGGAAGAGTATGATTGAACAAGATGGATTGC |
| | | ACGCAGGTTCTCCGGCCGCTTGGGTGGAGAGGC |
| | | TATTCGGCTATGACTGGGCACAACAGACAATCGG |
| | | CTGCTCTGATGCCGCCGTGTTCCGGCTGTCAGCG |
| | | CAGGGGCGCCCGGTTCTTTTTGTCAAGACCGACC |
| | | TGTCCGGTGCCCTGAATGAACTGCAAGACGAGG |
| | | CAGCGCGGCTATCGTGGCTGGCCACGACGGGCGT |
| | | TCCTTGCGCAGCTGTGCTCGACGTTGTCACTGAA |
| | | GCGGGAAGGGACTGGCTGCTATTGGGCGAAGTG |
| | | CCGGGGCAGGATCTCCTGTCATCTCACCTTGCTC |
| | | CTGCCGAGAAAGTATCCATCATGGCTGATGCAA |
| | | TGCGGCGGCTGCATACGCTTGATCCGGCTACCTG |
| | | CCCATTCGACCACCAAGCGAAACATCGCATCGA |
| | | GCGAGCACGTACTCGGATGGAAGCCGGTCTTGT |
| | | CGATCAGGATGATCTGGACGAAGAGCATCAGGG |
| | | GCTCGCGCCAGCCGAACTGTTCGCCAGGCTCAAG |
| | | GCGAGCATGCCCGACGGCGAGGATCTCGTCGTG |
| | | ACCCATGGCGATGCCTGCTTGCCGAATATCATGG |
| | | TGGAAAATGGCCGCTTTTCTGGATTCATCGACTG |
| | | TGGCCGGCTGGGTGTGGCGGACCGCTATCAGGA |
| | | CATAGCGTTGGCTACCCGTGATATTGCTGAAGAG |
| | | CTTGGCGGCGAATGGGCTGACCGCTTCCTCGTGC |
| | | TTTACGGTATCGCCGCTCCCGATTCGCAGCGCAT |
| | | CGCCTTCTATCGCCTTCTTGACGAGTTCTTCTGA |
| | | TAACTGTCAGACCAAGTTTACTCATATATACTTT |
| | | AGATTGATTTAAAACTTCATTTTTAATTTAAAAG |
| | | GATCTAGGTGAAGATCCTTTTTGATAATCTCATG |
| | | ACCAAAATCCCTTAACGTGAGTTTTCGTTCCACT |
| | | GAGCGTCAGACCCCGTAGAAAAGATCAAAGGAT |
| | | CTTCTTGAGATCCTTTTTTTCTGCGCGTAATCTGC |
| | | TGCTTGCAAACAAAAAAACCACCGCTACCAGCG |
| | | GTGGTTTGTTTGCCGGATCAAGAGCTACCAACTC |
| | | TTTTTCCGAAGGTAACTGGCTTCAGCAGAGCGCA |
| | | GATACCAAATACTGTTCTTCTAGTGTAGCCGTAG |
| | | TTAGGCCACCACTTCAAGAACTCTGTAGCACCGC |
| | | CTACATACCTCGCTCTGCTAATCCTGTTACCAGT |
| | | GGCTGCTGCCAGTGGCGATAAGTCGTGTCTTACC |
| | | GGGTTGGACTCAAGACGATAGTTACCGGATAAG |
| | | GCGCAGCGGTCGGGCTGAACGGGGGGTTCGTGC |
| | | ACACAGCCCAGCTTGGAGCGAACGACCTACACC |
| | | GAACTGAGATACCTACAGCGTGAGCTATGAGAA |
| | | AGCGCCACGCTTCCCGAAGGGAGAAAGGCGGAC |
| | | AGGTATCCGGTAAGCGGCAGGGTCGGAACAGGA |
| | | GAGCGCACGAGGGAGCTTCCAGGGGGAAACGCC |
| | | TGGTATCTTTATAGTCCTGTCGGGTTTCGCCACC |
| | | TCTGACTTGAGCGTCGATTTTTGTGATGCTCGTC |
| | | AGGGGGGCGGAGCCTATGGAAAAACGCCAGCAA |
| | | CGCGGCCTTTTTACGGTTCCTGGCCTTTTGCTGG |
| | | CCTTTTGCTCACATGTTCTTTCCTGCGTTATCCCC |
| | | TGATTCTGTGGATAACCGTATTACCGCCTTTGAG |
| | | TGAGCTGATACCGCTCGCCGCAGCCGAACGACC |
| | | GAGCGCAGCGAGTCAGTGAGCGAGGAAGCGGAA |
| | | GAGCGCCCAATACGCAAACCGCCTCTCCCCGCG |
| | | CGTTGGCCGATTCATTAATGCAGCTGGCACGAC |
| | | AGGTTTCCCGACTGGAAAGCGGGCAGTGAGCGC |
| | | AACGCAATTAATGTGAGTTAGCTCACTCATTAGG |
| | | CACCCCAGGCTTTACACTTTATGCTTCCGGCTCG |
| | | TATGTTGTGTGGAATTGTGAGCGGATAACAATTT |
| | | CACACAGGAAACAGCTATGACCATGATTACGCC |
| | | AGATTTAATTAAGGCCTTAATTAGG |
| 14 | GALT protein | Met Ser Arg Ser Gly Thr Asp Pro Gln Gln Arg Gln Gln Ala Ser Glu |
| | | Ala Asp Ala Ala Ala Ala Thr Phe Arg Ala Asn Asp His Gln His Ile |
| | | Arg Tyr Asn Pro Leu Gln Asp Glu Trp Val Leu Val Ser Ala His Arg |
| | | Met Lys Arg Pro Trp Gln Gly Gln Val Glu Pro Gln Leu Leu Lys Thr |
| | | Val Pro Arg His Asp Pro Leu Asn Pro Leu Cys Pro Gly Ala Ile Arg |
| | | Ala Asn Gly Glu Val Asn Pro Gln Tyr Asp Ser Thr Phe Leu Phe Asp |
| | | Asn Asp Phe Pro Ala Leu Gln Pro Asp Ala Pro Ser Pro Gly Pro Ser |
| | | Asp His Pro Leu Phe Gln Ala Lys Ser Ala Arg Gly Val Cys Lys Val |
| | | Met Cys Phe His Pro Trp Ser Asp Val Thr Leu Pro Leu Met Ser Val |
| | | Pro Glu Ile Arg Ala Val Val Asp Ala Trp Ala Ser Val Thr Glu Glu |
| | | Leu Gly Ala Gln Tyr Pro Trp Val Gln Ile Phe Glu Asn Lys Gly Ala |
| | | Met Met Gly Cys Ser Asn Pro His Pro His Cys Gln Val Trp Ala Ser |
| | | Ser Phe Leu Pro Asp Ile Ala Gln Arg Glu Glu Arg Ser Gln Gln Ala |
| | | Tyr Lys Ser Gln His Gly Glu Pro Leu Leu Met Glu Tyr Ser Arg Gln |
| | | Glu Leu Leu Arg Lys Glu Arg Leu Val Leu Thr Ser Glu His Trp Leu |
| | | Val Leu Val Pro Phe Trp Ala Thr Trp Pro Tyr Gln Thr Leu Leu Leu |
| | | Pro Arg Arg His Val Arg Arg Leu Pro Glu Leu Thr Pro Ala Glu Arg |
| | | Asp Asp Leu Ala Ser Ile Met Lys Lys Leu Leu Thr Lys Tyr Asp Asn |
| | | Leu Phe Glu Thr Ser Phe Pro Tyr Ser Met Gly Trp His Gly Ala Pro |
| | | Thr Gly Ser Glu Ala Gly Ala Asn Trp Asn His Trp Gln Leu His Ala |
| | | His Tyr Tyr Pro Pro Leu Leu Arg Ser Ala Thr Val Arg Lys Phe Met |
| | | Val Gly Tyr Glu Met Leu Ala Gln Ala Gln Arg Asp Leu Thr Pro Glu |
| | | Gln Ala Ala Glu Arg Leu Arg Ala Leu Pro Glu Val His Tyr His Leu |
| | | Gly Gln Lys Asp Arg Glu Thr Ala Thr Ile Ala |

### Numbered Embodiments

Embodiment 1. A nucleic acid molecule, comprising an adeno-associated virus (AAV) expression cassette, wherein the AAV expression cassette comprises, from 5′ to 3′:
   a 5′ AAV inverted terminal repeat (ITR);
   a promoter;
   a transgene encoding Galactose-1-Phosphate Uridylyltransferase (GALT); and
   a 3′ AAV ITR,
   wherein the transgene comprises a nucleic acid sequence having at least 90% identity to SEQ ID NO: 1.
Embodiment 2. The nucleic acid molecule of embodiment 1, wherein the transgene comprises the nucleic acid sequence of SEQ ID NO: 1.
Embodiment 3. The nucleic acid molecule of embodiment 1 or embodiment 2, wherein the promoter is operably linked to the transgene.
Embodiment 4. The nucleic acid molecule of embodiment 3, wherein the promoter comprises a CB6 promoter.
Embodiment 5. The nucleic acid molecule of embodiment 4, wherein the CB6 promoter comprises the nucleic acid sequence of SEQ ID NO: 2, or a sequence having at least 80%, 85%, 90%, 91%, 92%, 93%, 94%, 95%, 96%, 97%, 98%, 99%, or greater identity to SEQ ID NO: 2.
Embodiment 6. The nucleic acid molecule of any one of embodiments 1-5, wherein the AAV expression cassette comprises a rabbit globin polyadenylation sequence.
Embodiment 7. The nucleic acid molecule of embodiment 6, wherein the rabbit globin polyadenylation sequence comprises the nucleic acid sequence of SEQ ID NO: 3, or a sequence having at least 80%, 85%, 90%, 91%, 92%, 93%, 94%, 95%, 96%, 97%, 98%, 99%, or greater identity to SEQ ID NO: 3.
Embodiment 8. The nucleic acid molecule of any one of embodiments 1-7, wherein the AAV expression cassette comprises a CMV-IE enhancer.
Embodiment 9. The nucleic acid molecule of embodiment 8, wherein the CMV-IE enhancer comprises the nucleic acid sequence of SEQ ID NO: 4, or a sequence having at least 80%, 85%, 90%, 91%, 92%, 93%, 94%, 95%, 96%, 97%, 98%, 99%, or greater identity to SEQ ID NO: 4.
Embodiment 10. The nucleic acid molecule of any one of embodiments 1-9, wherein the AAV expression cassette comprises a Kozak sequence.
Embodiment 11. The nucleic acid molecule of embodiment 10, wherein the Kozak sequence comprises the nucleic acid sequence of SEQ ID NO: 5, or a sequence having at least 80%, 85%, 90%, 91%, 92%, 93%, 94%, 95%, 96%, 97%, 98%, 99%, or greater identity to SEQ ID NO: 5.
Embodiment 12. The nucleic acid molecule of any one of embodiments 1-11, wherein the 5′ AAV ITR sequence comprises the nucleic acid sequence of SEQ ID NO: 6, or a sequence having at least 80%, 85%, 90%, 91%, 92%, 93%, 94%, 95%, 96%, 97%, 98%, 99%, or greater identity to SEQ ID NO: 6.
Embodiment 13. The nucleic acid molecule of any one of embodiments 1-12, wherein the 3′ AAV ITR sequence comprises the nucleic acid sequence of SEQ ID NO: 7, or a sequence having at least 80%, 85%, 90%, 91%, 92%, 93%, 94%, 95%, 96%, 97%, 98%, 99%, or greater identity to SEQ ID NO: 7.
Embodiment 14. The nucleic acid molecule of any one of embodiments 1-13, wherein the AAV expression cassette comprises, from 5′ to 3″:
   the 5′ AAV ITR,
   a CMV-IE enhancer,
   a CB6 promoter,
   a Kozak sequence,
   the transgene encoding GALT,
   a rabbit globin polyadenylation sequence, and
   the 3′ AAV ITR.
Embodiment 15. The nucleic acid molecule of any one of embodiments 1-14, wherein the AAV expression cassette comprises the nucleic acid sequence of SEQ ID NO: 8, or a sequence having at least 80%, 85%, 90%, 91%, 92%, 93%, 94%, 95%, 96%, 97%, 98%, 99%, or greater identity to SEQ ID NO: 8.
Embodiment 16. A plasmid, comprising the nucleic acid molecule of any one of embodiments 1-15.
Embodiment 17. A cell, comprising the nucleic acid molecule of any one of embodiments 1-15, or the plasmid of embodiment 16.
Embodiment 18. A method of producing a recombinant adeno-associated virus (rAAV), the method comprising:
   contacting an AAV producer cell with the nucleic acid molecule of any one of embodiments 1-15, or the plasmid of embodiment 16.
Embodiment 19. A recombinant adeno-associated virus (rAAV) produced by the method of embodiment 18.
Embodiment 20. The rAAV of embodiment 19, wherein the rAAV comprises an AAV9 capsid protein, an AAV8 capsid protein and/or an AAVrh10 capsid protein.
Embodiment 21. The rAAV of embodiment 20, wherein the rAAV comprises an AAV9 capsid protein.
Embodiment 22. A recombinant adeno-associated virus (rAAV), comprising:
   an AAV9 capsid protein; and the nucleic acid molecule of any one of embodiments 1-15.
Embodiment 23. A recombinant adeno-associated virus (rAAV), comprising: an AAV9 capsid protein; and a nucleic acid molecule, comprising an AAV expression cassette, wherein the AAV expression cassette comprises, from 5′ to 3′:
   a 5′ AAV ITR comprising the nucleic acid sequence of SEQ ID NO: 6;
   a CMV-IE enhancer comprising the nucleic acid sequence of SEQ ID NO: 4;
   a CB6 promoter comprising the nucleic acid sequence of SEQ ID NO: 2;
   a Kozak sequence comprising the nucleic acid sequence of SEQ ID NO: 5;
   a transgene encoding GALT comprising the nucleic acid sequence of SEQ ID NO: 1;
   a rabbit globin polyadenylation sequence comprising the nucleic acid sequence of SEQ ID NO: 3; and
   a 3′ AAV ITR comprising the nucleic acid sequence of SEQ ID NO: 7.
Embodiment 24. A recombinant adeno-associated virus (rAAV), comprising: an AAV9 capsid protein; and a nucleic acid molecule, comprising an AAV expression cassette, wherein the AAV expression cassette comprises, from 5′ to 3′:
   a 5′ AAV ITR comprising the nucleic acid sequence of SEQ ID NO: 6, or a sequence having at least 80%, 85%, 90%, 91%, 92%, 93%, 94%, 95%, 96%, 97%, 98%, 99%, or greater identity to SEQ ID NO: 6;
   a CMV-IE enhancer comprising the nucleic acid sequence of SEQ ID NO: 4, or a sequence having at least 80%, 85%, 90%, 91%, 92%, 93%, 94%, 95%, 96%, 97%, 98%, 99%, or greater identity to SEQ ID NO: 4;
   a CB6 promoter comprising the nucleic acid sequence of SEQ ID NO: 2, or a sequence having at least 80%, 85%, 90%, 91%, 92%, 93%, 94%, 95%, 96%, 97%, 98%, 99%, or greater identity to SEQ ID NO: 2;
   a Kozak sequence comprising the nucleic acid sequence of SEQ ID NO: 5, or a sequence having at least 80%, 85%, 90%, 91%, 92%, 93%, 94%, 95%, 96%, 97%, 98%, 99%, or greater identity to SEQ ID NO: 5;
   a transgene encoding GALT comprising the nucleic acid sequence of SEQ ID NO: 1, or a sequence having at least 80%, 85%, 90%, 91%, 92%, 93%, 94%, 95%, 96%, 97%, 98%, 99%, or greater identity to SEQ ID NO: 1;
   a rabbit globin polyadenylation sequence comprising the nucleic acid sequence of SEQ ID NO: 3, or a sequence having at least 80%, 85%, 90%, 91%, 92%, 93%, 94%, 95%, 96%, 97%, 98%, 99%, or greater identity to SEQ ID NO: 3; and
   a 3′ AAV ITR comprising the nucleic acid sequence of SEQ ID NO: 7, or a sequence having at least 80%, 85%, 90%, 91%, 92%, 93%, 94%, 95%, 96%, 97%, 98%, 99%, or greater identity to SEQ ID NO: 7.
Embodiment 25. A recombinant adeno-associated virus (rAAV), comprising:
   an AAV9 capsid protein; and
   a nucleic acid molecule, comprising an AAV expression cassette, wherein the AAV expression cassette comprises the nucleic acid sequence of SEQ ID NO: 8.
Embodiment 26. A recombinant adeno-associated virus (rAAV), comprising:
   an AAV9 capsid protein; and
   a nucleic acid molecule, comprising an AAV expression cassette, wherein the AAV expression cassette comprises the nucleic acid sequence of SEQ ID NO: 8, or a sequence having at least 80%, 85%, 90%, 91%, 92%, 93%, 94%, 95%, 96%, 97%, 98%, 99%, or greater identity to SEQ ID NO: 8.
Embodiment 27. The rAAV of any one of embodiments 19-26, wherein the AAV expression cassette comprises an miR-1 binding site, an miR-133a binding site, and/or an miR-122 binding site.
Embodiment 28. The rAAV of any one of embodiments 19-27, wherein the rAAV is a self-complementary AAV.
Embodiment 29. The rAAV of any one of embodiments 19-27, wherein the rAAV is a single-stranded AAV.
Embodiment 30. A pharmaceutical composition, comprising:
   (a) the nucleic acid molecule of any one of embodiments 1-15, the plasmid of embodiment 16, the cell of embodiment 17, or the rAAV of any one of embodiments 19-29; and
   (b) a pharmaceutically acceptable carrier.
Embodiment 31. A method of expressing Galactose-1-Phosphate Uridylyltransferase (GALT) in a cell, comprising:
   contacting the cell with the nucleic acid molecule of any one of embodiments 1-15, the plasmid of embodiment 16, the rAAV of any one of embodiments 19-29, or the composition of embodiment 30, thereby expressing GALT in the cell.
Embodiment 32. A method of reducing the level of galactose-1-phosphate in a cell, comprising:
   contacting the cell with the nucleic acid molecule of any one of embodiments 1-15, the plasmid of embodiment 16, the rAAV of any one of embodiments 19-29, or the composition of embodiment 30, thereby reducing the level of galactose-1-phosphate in the cell.
Embodiment 33. The method of embodiment 31 or 32, wherein the contacting step is performed in vitro, ex vivo, or in vivo.
Embodiment 34. The method of embodiment 33, wherein the contacting step is performed in vivo in a subject in need thereof.
Embodiment 35. The method of embodiment 34, wherein the contacting step comprises administering a therapeutically effective amount of the nucleic acid molecule, the plasmid, the rAAV, or the composition to the subject.
Embodiment 36. A method of treating galactosemia in a subject in need thereof, comprising: administering to the subject a therapeutically effective amount of the nucleic acid molecule of any one of embodiments 1-15, the plasmid of embodiment 16, the cell of embodiment 17, the rAAV of any one of embodiments 19-29, or the composition of embodiment 30, thereby treating galactosemia in the subject.
Embodiment 37. The method of embodiment 35 or 36, wherein the nucleic acid molecule, the plasmid, the cell, the rAAV, or the composition is administered intravenously.
Embodiment 38. The method of any one of embodiments 35-37, wherein the method comprises administering a therapeutically effective amount of rAAV, wherein the therapeutically effective amount is in a range of 10¹⁰ genome copies to 10¹⁴ genome copies per kilogram.
Embodiment 39. The method of any one of embodiments 35-38, wherein the administration results in an increase in the level of GALT in a cell of the subject, as compared to a control subject having galactosemia who is not administered the nucleic acid molecule, the plasmid, the cell, the rAAV or the composition.
Embodiment 40. The method of any one of embodiments 35-39, wherein the administration results in a decrease in the level of galactose-1-phosphate in a cell of the subject, as compared to a control subject having galactosemia who is not administered the nucleic acid molecule, the plasmid, the cell, the rAAV or the composition.
Embodiment 41. The method of embodiment 39 or 40, wherein the level of GALT is increased and/or the level of galactose-1-phosphate is decreased in a brain cell, a liver cell, an ovarian cell, or a red blood cell (RBC).
Embodiment 42. The method of any one of embodiments 35-41, wherein the administration diminishes the severity of a symptom of galactosemia.
Embodiment 43. The method of any one of embodiments 35-42, wherein the administration delays the onset of a symptom of galactosemia.
Embodiment 44. The method of any one of embodiments 35-43, wherein the administration eliminates a symptom of galactosemia.
Embodiment 45. The method of any one of embodiments 42-44, wherein the symptom of galactosemia is liver dysfunction, kidney dysfunction, sepsis, brain edema, pseudotumor cerebri, feeding difficulty, reduction in growth, cataracts, speech disorder, language disorder, dyspraxia, developmental delay, attention deficit, learning delay, neurological impairment, behavioral disorder, emotional disorder, tremors, fertility dysfunction, primary ovarian insufficiency, anxiety, seizures, depression, attention deficit hyperactivity disorder (ADHD), dementia, or any combination thereof.
Embodiment 46. The method of any one of embodiments 36-45, wherein the galactosemia is classic type I galactosemia.
Embodiment 47. The method of any one of embodiments 34-46, wherein the subject is a human subject.
Embodiment 48. The method of embodiment 47, wherein the subject is a neonate or an infant.
Embodiment 49. The method of any one of embodiments 34-48, wherein the subject suffers from galactosemia.
Embodiment 50. The method of any one of embodiments 34-49, wherein the subject has been diagnosed with galactosemia.
Embodiment 51. The method of any one of embodiments 34-49, wherein the subject has one or more mutations in the GALT gene.
Embodiment 52. The method of any one of embodiments 34-51, wherein the subject is at a risk of developing at least one symptom of galactosemia.

What is claimed is:

1. A nucleic acid molecule, comprising an adeno-associated virus (AAV) expression cassette, wherein the AAV expression cassette comprises, from 5′ to 3′:
a 5′ AAV inverted terminal repeat (ITR);
a promoter;
a transgene encoding Galactose-1-Phosphate Uridylyltransferase (GALT); and
a 3′ AAV ITR,
wherein the transgene comprises a nucleic acid sequence having at least 90% identity to SEQ ID NO: 1.

2. The nucleic acid molecule of claim 1, wherein the transgene comprises the nucleic acid sequence of SEQ ID NO: 1.

3. The nucleic acid molecule of claim 1 or claim 2, wherein the promoter is operably linked to the transgene.

4. The nucleic acid molecule of claim 3, wherein the promoter comprises a CB6 promoter.

5. The nucleic acid molecule of claim 4, wherein the CB6 promoter comprises the nucleic acid sequence of SEQ ID NO: 2, or a sequence having at least 80%, 85%, 90%, 91%, 92%, 93%, 94%, 95%, 96%, 97%, 98%, 99%, or greater identity to SEQ ID NO: 2.

6. The nucleic acid molecule of any one of claims 1-5, wherein the AAV expression cassette comprises a rabbit globin polyadenylation sequence.

7. The nucleic acid molecule of claim 6, wherein the rabbit globin polyadenylation sequence comprises the nucleic acid sequence of SEQ ID NO: 3, or a sequence having at least 80%, 85%, 90%, 91%, 92%, 93%, 94%, 95%, 96%, 97%, 98%, 99%, or greater identity to SEQ ID NO: 3.

8. The nucleic acid molecule of any one of claims 1-7, wherein the AAV expression cassette comprises a CMV-IE enhancer.

9. The nucleic acid molecule of claim 8, wherein the CMV-IE enhancer comprises the nucleic acid sequence of SEQ ID NO: 4, or a sequence having at least 80%, 85%, 90%, 91%, 92%, 93%, 94%, 95%, 96%, 97%, 98%, 99%, or greater identity to SEQ ID NO: 4.

10. The nucleic acid molecule of any one of claims 1-9, wherein the AAV expression cassette comprises a Kozak sequence.

11. The nucleic acid molecule of claim 10, wherein the Kozak sequence comprises the nucleic acid sequence of SEQ ID NO: 5, or a sequence having at least 80%, 85%, 90%, 91%, 92%, 93%, 94%, 95%, 96%, 97%, 98%, 99%, or greater identity to SEQ ID NO: 5.

12. The nucleic acid molecule of any one of claims 1-11, wherein the 5′ AAV ITR sequence comprises the nucleic acid sequence of SEQ ID NO: 6, or a sequence having at least 80%, 85%, 90%, 91%, 92%, 93%, 94%, 95%, 96%, 97%, 98%, 99%, or greater identity to SEQ ID NO: 6.

13. The nucleic acid molecule of any one of claims 1-12, wherein the 3′ AAV ITR sequence comprises the nucleic acid sequence of SEQ ID NO: 7, or a sequence having at least 80%, 85%, 90%, 91%, 92%, 93%, 94%, 95%, 96%, 97%, 98%, 99%, or greater identity to SEQ ID NO: 7.

14. The nucleic acid molecule of any one of claims 1-13, wherein the AAV expression cassette comprises, from 5′ to 3′:
the 5′ AAV ITR,
a CMV-IE enhancer,
a CB6 promoter,
a Kozak sequence,
the transgene encoding GALT,
a rabbit globin polyadenylation sequence, and
the 3′ AAV ITR.

15. The nucleic acid molecule of any one of claims 1-14, wherein the AAV expression cassette comprises the nucleic acid sequence of SEQ ID NO: 8, or a sequence having at least 80%, 85%, 90%, 91%, 92%, 93%, 94%, 95%, 96%, 97%, 98%, 99%, or greater identity to SEQ ID NO: 8.

16. A plasmid, comprising the nucleic acid molecule of any one of claims 1-15.

17. A cell, comprising the nucleic acid molecule of any one of claims 1-15, or the plasmid of claim 16.

18. A method of producing a recombinant adeno-associated virus (rAAV), the method comprising:
contacting an AAV producer cell with the nucleic acid molecule of any one of claims 1-15, or the plasmid of claim 16.

19. A recombinant adeno-associated virus (rAAV) produced by the method of claim 18.

20. The rAAV of claim 19, wherein the rAAV comprises an AAV9 capsid protein, an AAV8 capsid protein and/or an AAVrh10 capsid protein.

21. The rAAV of claim 20, wherein the rAAV comprises an AAV9 capsid protein.

22. A recombinant adeno-associated virus (rAAV), comprising:
an AAV9 capsid protein; and the nucleic acid molecule of any one of claims 1-15.

23. A recombinant adeno-associated virus (rAAV), comprising: an AAV9 capsid protein; and a nucleic acid molecule, comprising an AAV expression cassette, wherein the AAV expression cassette comprises, from 5′ to 3′:
a 5′ AAV ITR comprising the nucleic acid sequence of SEQ ID NO: 6;
a CMV-IE enhancer comprising the nucleic acid sequence of SEQ ID NO: 4;
a CB6 promoter comprising the nucleic acid sequence of SEQ ID NO: 2;
a Kozak sequence comprising the nucleic acid sequence of SEQ ID NO: 5;
a transgene encoding GALT comprising the nucleic acid sequence of SEQ ID NO: 1;
a rabbit globin polyadenylation sequence comprising the nucleic acid sequence of SEQ ID NO: 3; and
a 3′ AAV ITR comprising the nucleic acid sequence of SEQ ID NO: 7.

24. A recombinant adeno-associated virus (rAAV), comprising: an AAV9 capsid protein; and a nucleic acid molecule, comprising an AAV expression cassette, wherein the AAV expression cassette comprises, from 5′ to 3′:
a 5′ AAV ITR comprising the nucleic acid sequence of SEQ ID NO: 6, or a sequence having at least 80%, 85%, 90%, 91%, 92%, 93%, 94%, 95%, 96%, 97%, 98%, 99%, or greater identity to SEQ ID NO: 6;
a CMV-IE enhancer comprising the nucleic acid sequence of SEQ ID NO: 4, or a sequence having at least 80%, 85%, 90%, 91%, 92%, 93%, 94%, 95%, 96%, 97%, 98%, 99%, or greater identity to SEQ ID NO: 4;
a CB6 promoter comprising the nucleic acid sequence of SEQ ID NO: 2, or a sequence having at least 80%, 85%, 90%, 91%, 92%, 93%, 94%, 95%, 96%, 97%, 98%, 99%, or greater identity to SEQ ID NO: 2;
a Kozak sequence comprising the nucleic acid sequence of SEQ ID NO: 5, or a sequence having at least 80%, 85%, 90%, 91%, 92%, 93%, 94%, 95%, 96%, 97%, 98%, 99%, or greater identity to SEQ ID NO: 5;
a transgene encoding GALT comprising the nucleic acid sequence of SEQ ID NO: 1, or a sequence having at least 80%, 85%, 90%, 91%, 92%, 93%, 94%, 95%, 96%, 97%, 98%, 99%, or greater identity to SEQ ID NO: 1;
a rabbit globin polyadenylation sequence comprising the nucleic acid sequence of SEQ ID NO: 3, or a sequence having at least 80%, 85%, 90%, 91%, 92%, 93%, 94%, 95%, 96%, 97%, 98%, 99%, or greater identity to SEQ ID NO: 3; and
a 3′ AAV ITR comprising the nucleic acid sequence of SEQ ID NO: 7, or a sequence having at least 80%, 85%, 90%, 91%, 92%, 93%, 94%, 95%, 96%, 97%, 98%, 99%, or greater identity to SEQ ID NO: 7.

25. A recombinant adeno-associated virus (rAAV), comprising:
an AAV9 capsid protein; and
a nucleic acid molecule, comprising an AAV expression cassette, wherein the AAV expression cassette comprises the nucleic acid sequence of SEQ ID NO: 8.

26. A recombinant adeno-associated virus (rAAV), comprising:
an AAV9 capsid protein; and
a nucleic acid molecule, comprising an AAV expression cassette, wherein the AAV expression cassette comprises the nucleic acid sequence of SEQ ID NO: 8, or a sequence having at least 80%, 85%, 90%, 91%, 92%, 93%, 94%, 95%, 96%, 97%, 98%, 99%, or greater identity to SEQ ID NO: 8.

27. The rAAV of any one of claims 19-26, wherein the AAV expression cassette comprises an miR-1 binding site, an miR-133a binding site, and/or an miR-122 binding site.

28. The rAAV of any one of claims 19-27, wherein the rAAV is a self-complementary AAV.

29. The rAAV of any one of claims 19-27, wherein the rAAV is a single-stranded AAV.

30. A pharmaceutical composition, comprising:
(a) the nucleic acid molecule of any one of claims 1-15, the plasmid of claim 16, the cell of claim 17, or the rAAV of any one of claims 19-29; and
(b) a pharmaceutically acceptable carrier.

31. A method of expressing Galactose-1-Phosphate Uridylyltransferase (GALT) in a cell, comprising:
contacting the cell with the nucleic acid molecule of any one of claims 1-15, the plasmid of claim 16, the rAAV of any one of claims 19-29, or the composition of claim 30, thereby expressing GALT in the cell.

32. A method of reducing the level of galactose-1-phosphate in a cell, comprising:
contacting the cell with the nucleic acid molecule of any one of claims 1-15, the plasmid of claim 16, the rAAV of any one of claims 19-29, or the composition of claim 30, thereby reducing the level of galactose-1-phosphate in the cell.

33. The method of claim 31 or 32, wherein the contacting step is performed in vitro, ex vivo, or in vivo.

34. The method of claim 33, wherein the contacting step is performed in vivo in a subject in need thereof.

35. The method of claim 34, wherein the contacting step comprises administering a therapeutically effective amount of the nucleic acid molecule, the plasmid, the rAAV, or the composition to the subject.

36. A method of treating galactosemia in a subject in need thereof, comprising: administering to the subject a therapeutically effective amount of the nucleic acid molecule of any one of claims 1-15, the plasmid of claim 16, the cell of claim 17, the rAAV of any one of claims 19-29, or the composition of claim 30, thereby treating galactosemia in the subject.

37. The method of claim 35 or 36, wherein the nucleic acid molecule, the plasmid, the cell, the rAAV, or the composition is administered intravenously.

38. The method of any one of claims 35-37, wherein the method comprises administering a therapeutically effective amount of rAAV, wherein the therapeutically effective amount is in a range of 10¹⁰ genome copies to 10¹⁴ genome copies per kilogram.

39. The method of any one of claims 35-38, wherein the administration results in an increase in the level of GALT in a cell of the subject, as compared to a control subject having galactosemia who is not administered the nucleic acid molecule, the plasmid, the cell, the rAAV or the composition.

40. The method of any one of claims 35-39, wherein the administration results in a decrease in the level of galactose-1-phosphate in a cell of the subject, as compared to a control subject having galactosemia who is not administered the nucleic acid molecule, the plasmid, the cell, the rAAV or the composition.

41. The method of claim 39 or 40, wherein the level of GALT is increased and/or the level of galactose-1-phosphate is decreased in a brain cell, a liver cell, an ovarian cell, or a red blood cell (RBC).

42. The method of any one of claims 35-41, wherein the administration diminishes the severity of a symptom of galactosemia.

43. The method of any one of claims 35-42, wherein the administration delays the onset of a symptom of galactosemia.

44. The method of any one of claims 35-43, wherein the administration eliminates a symptom of galactosemia.

45. The method of any one of claims 42-44, wherein the symptom of galactosemia is liver dysfunction, kidney dysfunction, sepsis, brain edema, pseudotumor cerebri, feeding difficulty, reduction in growth, cataracts, speech disorder, language disorder, dyspraxia, developmental delay, attention deficit, learning delay, neurological impairment, behavioral disorder, emotional disorder, tremors, fertility dysfunction, primary ovarian insufficiency, anxiety, seizures, depression, attention deficit hyperactivity disorder (ADHD), dementia, or any combination thereof.

46. The method of any one of claims 36-45, wherein the galactosemia is classic type I galactosemia.

47. The method of any one of claims 34-46, wherein the subject is a human subject.

48. The method of claim 47, wherein the subject is a neonate or an infant.

49. The method of any one of claims 34-48, wherein the subject suffers from galactosemia.

50. The method of any one of claims 34-49, wherein the subject has been diagnosed with galactosemia.

51. The method of any one of claims 34-49, wherein the subject has one or more mutations in the GALT gene.

52. The method of any one of claims 34-51, wherein the subject is at a risk of developing at least one symptom of galactosemia.
